(19) United States
(12) Patent Application Publication
(10) Pub. No.: US 2017/0037475 A1
(43) Pub. Date: Feb. 9, 2017

(54) GENETIC MARKERS ASSOCIATED WITH ASD AND OTHER CHILDHOOD DEVELOPMENTAL DELAY DISORDERS

(71) Applicant: Lineagen, Inc., Salt Lake City, UT (US)

(72) Inventors: Karen HO, Salt Lake City, UT (US); Charles HENSEL, Salt Lake City, UT (US)

(21) Appl. No.: 15/302,696

(22) PCT Filed: Apr. 9, 2015
(86) PCT No.: PCT/US15/25201

§ 371 Date: Oct. 7, 2016

Related U.S. Application Data
(60) Provisional application No. 61/977,462, filed on Apr. 9, 2014.

(51) Int. Cl. C12Q 1/68

(52) U.S. Cl. CPC C12Q 1/6883; C12Q 2600/156; C12Q 2600/158

(57) ABSTRACT

The present invention relates generally to genetic markers for duplication and/or deletion syndromes, such as Wolf-Hirschhorn syndrome (WHS), in particular to copy number variant genetic markers for selecting a patient for therapy for the particular therapy, or predicting the response of a subject to a particular therapy.

### CROSS REFERENCE TO RELATED APPLICATION

This application claims the benefit of priority from U.S. Provisional Application Ser. No. 61/977,462, filed Apr. 9, 2014, the disclosure of which is incorporated by reference herein in its entirety.

### STATEMENT REGARDING SEQUENCE LISTING

The Sequence Listing associated with this application is provided in text format in lieu of a paper copy, and is hereby incorporated by reference into the specification in its entirety for all purposes. The name of the text file containing the Sequence Listing is LINE_006_01 WO. The text file is 12.2 MB, was created on Apr. 9, 2015, and is being submitted electronically via EFS-Web.

### BACKGROUND OF THE INVENTION

Developmental delay disorders are an ever growing group of disorders. Many disorders of childhood development are associated with aberrant copy number (i.e., gain or loss of copy number) of a particular sub-chromosomal region. Developmental delay disorders encompass a wide range of symptoms, skills, and levels of impairment, or disability, that children with the disorder can have. Autism spectrum disorders are closely related to developmental delay disorders. They comprise a spectrum of complex, heterogeneous, behaviorally-defined group of disorders characterized by impairments in social interaction and communication as well as by repetitive and stereotyped behaviors and interests.

Genetic factors play a substantial role in disorders of childhood development (Abrahams B S, Geschwind D H. Advances in autism genetics: on the threshold of a new neurobiology. Nat Rev Genet 2008; 9:341-55; Matsunami et al. Identification of rare DNA sequence variants in high-risk autism families and their prevalence in a large case/control population. Molecular Autism 5:5 (2014); Matsunami et al. Identification of rare recurrent copy number variants in high-risk autism families and their prevalence in a large ASD population. PLOS one 8(1):e52239 (2013)). Genetic mutations and chromosomal abnormalities that play a role in disorders of childhood development may be deletion or duplication variants, including copy number variants (CNV) or single nucleotide variants.

While there is no known medical treatment for many childhood development disorders, some success has been reported for early intervention with behavioral therapies. Identification of genetic markers and biomarkers for disorders of childhood development would allow earlier identification of the disease. Genetic evaluation of subjects suffering from childhood development disorder may also help predict out comes of both pharmacologic and behavioral therapies. Thus, there is an urgent need for a method of reliably identifying subjects with disorders of childhood development.

Wolf-Hirschhorn Syndrome (WHS) is a developmental delay disorder that exhibits high variability of its associated features. These features include the following: characteristic facial dysmorphology, intellectual disability, growth deficiency, seizures, congenital heart disease, kidney dysfunction, scoliosis, and oligodontia, and others.

WHS is a rare, multi-genetic disorder that results from the deletion of contiguous genes in the distal region of the short arm of chromosome 4. Presentation of the disorder includes: intellectual disability, failure to thrive, seizures, and a characteristic facies. The degree to which these “classic” features as well as other co-morbid conditions present themselves in each patient can vary significantly, thereby requiring that the medical management of this disorder be tailored to an individual's needs. Without the benefit of genetic correlation studies of this syndrome, standard medical care for Wolf-Hirschhorn patients means the running of expensive and sometimes invasive medical tests for each patient in order to determine the best course of action.

There is an increasing body of biochemical and genetic evidence suggesting that mitochondrial dysfunction is involved in the pathology of autism (Legido el al. (2013). Seminars in Pediatric Neurology 20, pp. 163-175), as well as other types of developmental delay (DD) disorders. However, not all individuals with ASD or DD display indicators of oxidative stress or mitochondrial dysfunction. Associated with ASD etiology is a strong genetic component; over 800 genetic changes have been proposed to be involved in the causes for ASD (lossifov et al. (2012) Neuron 74, pp. 285-299). Determination of the genetic changes associated with ASD features in individuals may determine the appropriateness of mitochondrial therapies on an individual basis.

### SUMMARY OF THE INVENTION

In one aspect of the invention, the present invention provides a method for determining the presence or absence of a deletion or duplication syndrome in a subject. For example, in one embodiment, a method for determining the presence or absence of a deletion or duplication syndrome associated with developmental delay in a subject is provided, wherein the method provides high subchromosomal resolution of the deletion and/or duplication. In one embodiment, the deletion or duplication syndrome is selected from one or more of the deletion or duplication syndromes set forth at Table A and/or Table B. In a further embodiment, the subject is selected for therapy of the deletion or duplication syndrome if the CNV is present, and is at least about 500 bases in length.

The method in one embodiment comprises probing a sample obtained from the subject for the presence or absence of one or more copy number variants (CNVs) associated with the chromosomal deletion or duplication syndrome, and if the CNV is present, optionally analyzing the size of the deletion or duplication of at least one CNV. In one embodiment, the probing step comprises mixing the sample with five or more oligonucleotides that are substantially complementary to portions of the genomic DNA sequence associated with the deletion or duplication syndrome under conditions suitable for hybridization of the five or more oligonucleotides to their complements or substantial complements; detecting whether hybridization occurs between the five or more oligonucleotides to their complements or substantial complements, or a subset thereof and obtaining hybridization values of the sample based on the detecting step.

The determination of whether the CNV is present or absent, in one embodiment, comprises comparing the hybridization values of the sample to reference hybridization value(s) from at least one training set comprising hybridization value(s) from a sample that is positive for the one or more CNVs, or hybridization value(s) from a sample that is negative for the one or more CNVs. In one embodiment, the comparing step comprises determining a correlation between the hybridization values obtained from the sample and the hybridization value(s) from the at least one training set (which may be included in a database of values or a sample training set). A determination is then made regarding the presence or absence of the at least one CNV followed by an assessment of whether the subject has the chromosomal deletion or duplication syndrome.

In one embodiment, the sample comprises restriction digested double stranded DNA obtained from genomic DNA fragments; restriction digested single stranded DNA obtained from genomic DNA fragments; amplified restriction digested genomic DNA single stranded fragments; amplified restriction digested genomic DNA double stranded fragments; or a combination thereof. In a further embodiment, the sample is free of histone proteins. In even a further embodiment, the amplified restriction digested genomic DNA single stranded fragments comprise a detectable label chemically attached to individual single stranded fragments. In yet a further embodiment, the amplified restriction digested genomic DNA single stranded fragments further comprise adapter sequences. In one embodiment, the adapter sequences are introduced via adapter-specific primers.

In one embodiment, the subject is identified as at risk for a clinical manifestation of the deletion or duplication syndrome if the size of the deletion is greater than or equal to 500 bp. Accordingly, if the size of the deletion or duplication is greater than or equal to 500 bp, the subject is selected for treatment of the deletion or duplication syndrome. Alternatively or additionally, depending on the size of the deletion or duplication, a prediction is made regarding whether the subject will respond to treatment for the deletion or duplication syndrome, for example, treatment of a clinical manifestation of the deletion or duplication syndrome.

The probing step in one embodiment comprises a DNA hybridization assay with oligonucleotides specific for DNA sequences associated with the one or more CNVs. The probing step comprises in one embodiment, polymerase chain reaction (PCR), a microarray assay, a NanoString assay (e.g., nCounter CNV Analysis), a sequencing assay (for example high throughput sequencing, single molecule sequencing, next-generation sequencing, etc.) or a combination thereof.

In one embodiment, the deletion or duplication syndrome is a syndrome wherein the chromosomal deletion or duplication is of a varying length. In one embodiment, the deletion syndrome is selected from the group consisting of Wolf-Hirshhorn (4p) syndrome, 22q11.2 deletion syndrome (DiGeorge syndrome), and 1p36 deletion syndrome. In one embodiment, the duplication syndrome is selected from the group consisting of 1q21.1 duplication syndrome, 8p23.1 duplication syndrome and chromosome 15q duplication syndrome. Where the deletion or duplication syndrome is a syndrome of chromosomal deletion or duplication is of a varying length, the method for selecting the subject for therapy of the syndrome, in one embodiment, comprises measuring the size of the CNV.

In a further embodiment, if the subject is diagnosed with the deletion or duplication syndrome, and is further selected for treatment, the subject is treated for a clinical manifestation of the deletion or duplication syndrome selected from congenital heart disease, seizure, renal disease, intellectual disability, developmental delay, vision loss, blindness, or other condition affecting ears, skin, teeth, or skeletal development; or a combination thereof.

In one embodiment, the deletion syndrome is Wolf-Hirshhorn (4p) syndrome (WHS) and the subject is selected for treatment of a clinical manifestation of WHS, if the CNV at chromosome 4p is greater than 500 bases, greater than 1,000 bases, greater than 100,000 bases, greater than 500,000 bases, greater than 1 Mb, greater than 5 Mb, greater than 10 Mb, or greater than 1 Mb. In one embodiment, the method further comprises treating the subject for the clinical manifestation of WHS. In a further embodiment, the method comprises treating the subject for congenital heart disease.

In yet another aspect of the invention, a method for selecting a subject for treatment of status epilepticus or for predicting the response of a subject to treatment of status epilepticus is provided. In one embodiment, the method comprises detecting in a genetic sample from the subject the presence or absence of a copy number variant (CNV) associated with Wolf-Hirshhom (4p-) syndrome; and detecting the presence or absence in the genetic sample a second CNV selected from the CNVs provided in Table 3, 4, 8-10, 12 and/or 13. In a further embodiment, the method comprises selecting the subject for treatment of status epilepticus if the first and second CNVs are detected.

In a further embodiment, the method comprises detecting the first and second CNVs using two or more sets of oligonucleotides, wherein each set of oligonucleotides is complementary or substantially complementary to at least a portion of the CNV associated with Wolf Hirshhorn (4p-) syndrome, or a CNV provided in Table 3, 4, 8-10, 12 and/or 13. In a yet further embodiment, the two or more sets of oligonucleotides each comprises from about 1 to about 100, or from about 2 to about 75, or from about 5 to 50, or from about 10 two about 25, or from about 15 to about 20 oligonucleotides. In another embodiment, the two or more sets of oligonucleotides comprises about 5, about 10, about 15, about 20, about 25, about 30, about 35, about 40, about 45, or about 50 oligonucleotides. In one embodiment, the two or more sets of oligonucleotides are present on an array, such as a high density microarray. In yet another embodiment, the presence or absence of the CNVs are determined via a nucleic acid hybridization assay selected from a PCR based assay, a NanoString assay (e.g., nCounter CNV Analysis) or a sequencing assay (for example high throughput sequencing, single molecule sequencing, next-generation sequencing, etc.).

In another embodiment, the one or more CNVs are associated with one or more mitochondrial associated genes, for example, one or more of the genes set forth in Table 15, herein. Accordingly, the present invention provides methods for determining the presence or absence of a mitochondrial related disorder, and methods for predicting the likelihood of whether a subject will develop such a disorder, e.g., by probing for one or more CNVs that affect mitochondrial associated genes.

In another embodiment, a method for selecting a subject for mitochondrial therapy is provided. In one embodiment, the method comprises probing a genetic sample from the subject for the presence or absence of at least one copy number variant (CNV) associated with a mitochondrial gene, for example a gene set forth in Table 15. In one embodiment, the probing step comprises mixing the sample with five or more oligonucleotides that are substantially complementary to portions of the genomic DNA sequence associated with the CNV under conditions suitable for hybridization of the five or more oligonucleotides to their complements or substantial complements; detecting whether hybridization occurs between the five or more oligonucleotides to their complements or substantial complements, or a subset thereof and obtaining hybridization values of the sample based on the detecting step. The determination of whether the CNV is present or absent, in one embodiment, comprises comparing the hybridization values of the sample to reference hybridization value(s) from at least one training set comprising hybridization value(s) from a sample that is positive for the one or more CNVs, or hybridization value(s) from a sample that is negative for the one or more CNVs. In one embodiment, the comparing step comprises determining a correlation between the hybridization values obtained from the sample and the hybridization value(s) from the at least one training set (which may be included in a database of values or a sample training set). A determination is then made regarding the presence or absence of the at least one CNV followed by an assessment of whether the subject has the chromosomal deletion or duplication syndrome. The subject is then selected or not-selected for therapy based on the assessment of whether the syndrome is present.

In a further embodiment, if the CNV genetic marker is detected, the subject is selected for mitochondrial therapy and is administered mitochondrial therapy. The mitochondrial therapy, in one embodiment, is selected from an antioxidant, oxygen, arginine, Coenzyme Q10, idebenone, benzoquinone therapeutics (e.g., alpha-tocotrienol quinone (EPI-743) (Edison Pharmaceuticals)), creatine, lipoic acid, dichloroacetate (DCA), citrulline, or a combination thereof. In a further embodiment, if the patient is selected for mitochondrial therapy based on the results of the CNV analysis, the method comprises treating the subject with EPI-743.

In one embodiment, the method for determining whether a subject has a deletion or duplication syndrome (and optionally selecting the subject for treatment of the syndrome) comprising probing for the presence or absence in the genetic sample from the subject for 1, 2, 10, 15, 20, 25, 30, 40, 50, 60, 70, 80, 90, 100, 150, 200, or more CNVs. For example, in the case of a mitochondrial related deletion or duplication disorder, one or more of the CNVs in the genes set forth in Table 15 can be probed for. In another embodiment, the method comprises detecting in the genetic sample from the subject the presence of from 1 to 100, from 2 to 75, from 5 to 50, or from 10 to 25 CNVs. In one embodiment, the method comprises selecting the subject for therapy or predicting that the subject will respond to a therapy if the presence of at least 2, at least 5, at least 10, at least 25, or at least 50 of the CNVs are detected. In one embodiment, the at least one CNV comprises a copy number duplication CNV. In another embodiment, the at least one CNV comprises a copy number deletion CNV. In another embodiment, at least two CNVs are detected, and the at least two CNVs comprise a copy number deletion CNV and a copy number duplication CNV. In one embodiment, the at least one CNV is between about 400 base pairs (bp) to about 250 mega base pairs (Mb), between about 500 bp and 1 Mb, between about 500 bp and about 100 Mb, between about 500 bp and 500,000 bp, between about 500 bp and about 100,000 bp, between about 2 Mb and about 80 Mb, between about 5 Mb and about 40 Mb, or between about 10 Mb and about 20 Mb. The CNV(s) of the one or more mitochondrial associated genes, in one embodiment, is detected using a nucleic acid hybridization assay, for example a PCR based assay, a NanoString assay (e.g., nCounter CNV Analysis) or a sequencing assay (for example high throughput sequencing, single molecule sequencing, next-generation sequencing, etc.).

In one embodiment, the one or more sets of oligonucleotides used to interrogate a sample for whether one or more CNVs are present, are included on an array, such as a high density microarray. See, for example, Manning et al., ACMG CMA Practice Guidelines 2011, incorporated herein by reference in its entirety. In one embodiment, the probes on the array are selected from the probes set forth in the accompanying sequence listing, and correspond to the genome positions set forth in Table 14 from U.S. Provisional Application 61/977,462 and Table 14 from International PCT Publication No. 2014/055915, the disclosure of each of which is incorporated by reference in their entireties.

In another embodiment, the method for selecting a subject for a mitochondrial therapy, or for predicting the response of a subject to a mitochondrial therapy comprises determining the mitochondrial function affected by the one or more mitochondrial disease-associated genes associated with the CNV. In a further embodiment, the subject is treated with a mitochondrial therapy, and the mitochondrial therapy is selected based on the mitochondrial function of the one or more mitochondrial disease-associated genes. In a further embodiment, the mitochondrial function is associated with electron transport or regulation of oxidative stress. In one embodiment, the subject was previously diagnosed with an autism spectrum disorder.

In another embodiment, where a CNV is detected that affects one or more glutamergic or GABAergic signaling genes, methods are provided for determining whether the CNV is present in a subject's sample, and if present, a method is provided for selecting the subject for treatment with a drug targeting a glutamate receptor or a GABA receptor, or a method is provided for predicting the response of a subject to treatment with a drug targeting a glutamate receptor or a GABA receptor. For example, in one embodiment, the method comprising detecting in a genetic sample from the subject the presence or absence of a copy number variant (CNV), wherein the CNV is a CNV affecting one or more glutamatergic or GABAergic signaling genes, and selecting the subject for treatment or predicting that the subject will respond to treatment if the CNV is detected. The determination of whether the CNV is present or absent, in one embodiment, comprises comparing the hybridization values of the sample to reference hybridization value(s) from at least one training set comprising hybridization value(s) from a sample that is positive for the CNV, or hybridization value(s) from a sample that is negative for the CNV (such values may be stored in a database). In one embodiment, the comparing step comprises determining a correlation between the hybridization values obtained from the sample and the hybridization value(s) from the at least one training set. A determination is then made regarding the presence or absence of the at least one CNV.

In a further embodiment, the method comprises treating the subject with a glutamate receptor agonist or antagonist or a GABA receptor agonist or antagonist. In a further embodiment, the method comprises determining the effect of the CNV on the excitatory or inhibitory activity of the subject's neurons. In a further embodiment, the method comprises administering to the subject a receptor agonist if the effect of the CNV is an inhibitory effect. In another embodiment, the method comprises administering to the subject a receptor antagonist if the effect of the CNV is an excitatory effect.

### BRIEF DESCRIPTION OF THE DRAWINGS

FIG. 1: Workflow for CNV analysis for samples analyzed on the custom array. The same process was used for both CNAM and PennCNV analyses. All samples used for CNV analysis in this study had to meet the quality control measures described. Only unrelated cases and controls were used for the final statistical analysis.

FIG. 2: Manhattan plot of CNVs called both by PennCNV and CNAM. Association statistics across all regions covered on the Illumina custom array are shown. Since the array used was not a genome-wide array, the width of each chromosome on the plot is not proportional to the chromosome length. Adjacent chromosomes are separated by tick marks.

FIG. 3. UCSC Genome browser view of CNVs in the NRXN1 region. CNVs observed in the vicinity of the NRXN1-alpha transcription start site are shown. Note that most CNVs observed in ASD patients include exon 1 of NRXN1-alpha while only 1 control CNV extends into exon 1. Produced with custom tracks listing CNV calls and uploaded to the genome.ucsc.edu website.

FIG. 4. UCSC Genome Browser View of CNVs in the GABR Region on chromosome 15q12. Duplications were called by both PennCNV and by CNAM in this region, however the number of duplications called by each program differed, with many additional duplications called by CNAM. Produced with custom tracks listing CNV calls and uploaded to the genome.ucsc.edu website.

FIG. 5 is a graph of the number of clinical features exhibited by subjects as a function of deletion size in base pairs.

FIG. 6 is a graph of clinical features exhibited by subjects as a function of the number of genes in 4p deletion.

FIG. 7 is a graph showing the correlation between WHS deletion location and seizures. Those individuals who do not have seizures are shown with an asterisk (*). These individuals all have interstitial deletions that do not encompass the terminal region of the 4p chromosome. All other individuals report having significant numbers of seizures, especially throughout childhood. The boxed region of the chromosome ideogram (top part of figure) shows the chromosomal locations of all deletions illustrated with the bars in the graph below. 35 subjects with pure deletions are shown, with the two critical regions necessary for WHS shown for reference (labeled WHS Critical Region 1 and 2).

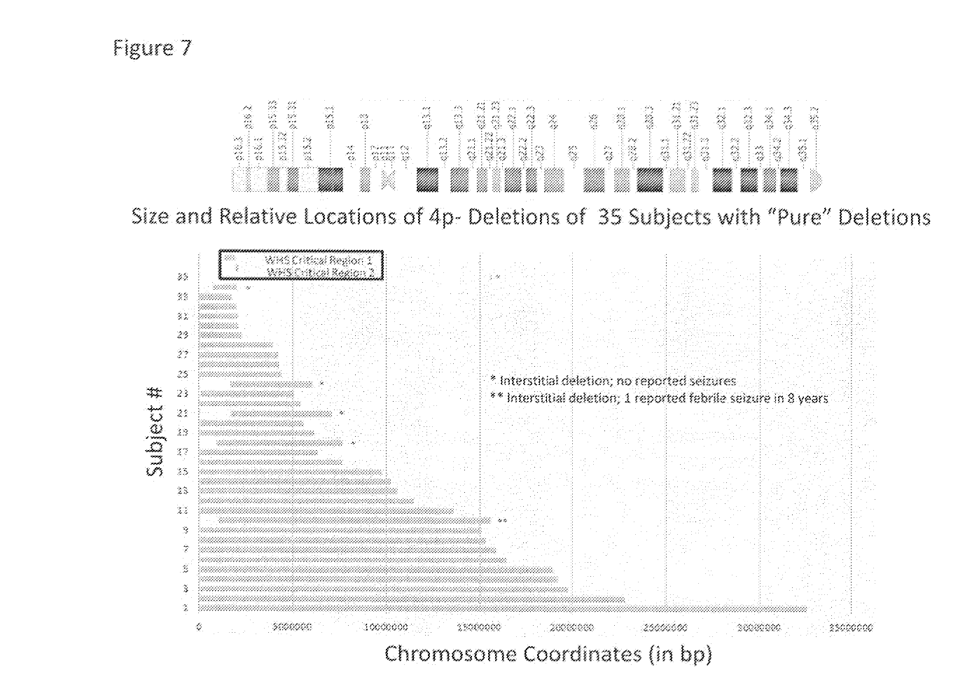

FIG. 8 illustrates that CMA data can be correlated with a specific type of clinical manifestation, in this case, congenital heart disease. Black bars indicate subjects with congenital heart disease. Gray bars represent subjects without congenital heart disease.

FIG. 9 shows that subjects with multiple CNV findings were more likely to have status epilepticus than subjects with only the 4p-deletion. Each horizontal bar on the graph represents the size and location of a subject's 4p-deletion as detected by the custom microarray provided herein. Black bars indicate subjects with status epilepticus. Gray bars represent subjects without status epilepticus.

### DETAILED DESCRIPTION OF THE INVENTION

The present invention relates generally to genetic markers for developmental delay disorders, and specifically, mitochondrial disorders, disorders associated with chromosomal duplications or chromosomal deletions (for example, chromosomal duplications or chromosomal deletions of mitochondrial associated genes). In particular, in one embodiment, the present copy number variant (CNV) genetic markers provide a diagnostic yield (the percentage of individuals with the diagnosis of the disorder that will have an abnormal genetic test result; equal to sensitivity) of at least about 10-12%, for example at least about 20%-40%, e.g., 25%-35%. In contrast, generic chromosomal microarray technologies currently available are expected to remain in the 5%-7% diagnostic yield range for the developmental disorder portion of these microarrays, or karyotype/FISH assay (that is, 5-7% of the individuals with the disorder that are tested with current technologies will have an abnormal result). Thus, in one embodiment, the present invention represents a 2× increase (5% to more than 10%) in specific diagnostic yield over current diagnostic platforms. In one embodiment, the practice of the present invention employs conventional methods of microbiology, molecular biology, recombinant DNA technique, chemical syntheses, chemical analyses, pharmaceutical preparation, formulation, and delivery, and treatment of patients, within the skill of the art, many of which are described below for the purpose of illustration. Such techniques are explained fully in the literature. See, e.g., Current Protocols in Protein Science, Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used in this specification and the appended claims, the singular forms “a,” “an” and “the” include plural references unless the content clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

Chromosomal duplication and deletion syndromes are often associated with developmental delay. The present invention provides a means for determining whether a subject's genomic DNA includes a copy number variant (“CNV”) at one or more chromosomal locations. For example, in one embodiment, the present invention provides one or more oligonucleotides that specifically hybridize to chromosomal regions set forth in Tables A and B, below, in order to determine whether a subject has a copy number variant in the particular region(s).

TABLE A
Autosomal Copy Number Variations

| Chromosomal | |
|---|---|
| Location | Associated condition/clinical features |
| 1p36 | 1p36 deletion syndrome |
| 1q21 | 1q21 deletion or duplication syndrome |
| 1q41q42 | 1q41q42 deletion syndrome |
| 1q43q44 | 1q43q44 deletion or duplication syndrome |
| 2p16.3 (NRXN1) | Neurodevelopmental disorder/autism spectrum |
| | disorder |
| 2p16.1p15 | 2p16.1p15 deletion syndrome |
| 2q21.1 | Neurodevelopmental disorder/autism spectrum |
| | disorder |
| 2q23.1 (MBD5) | Intellectual disability and seizures |
| 2q24.2 (SLC4A10) | Neurodevelopmental disorder/autism spectrum |
| | disorder |
| 2q33.1 | 2q33.1 deletion syndrome |
| 2q33.3q35 | Autism spectrum disorder |
| 2q37 (HDAC4) | 2q37.3 deletion syndrome |
| 3p26.3 (CNTN4) | Autism spectrum disorder |
| 3p14.1 (FOXP1) | 3p interstitial deletion syndrome |
| 3q29 | 3q29 deletion or duplication syndrome |
| 4p16.3 | Wolf-Hirschhorn syndrome (4p- syndrome) |
| 4p16.1 | Proximal 4p deletion syndrome |
| 4q32qter | Autism spectrum disorder |
| 4q35 | Neurodevelopmental disorder, autism spectrum |
| | disorder, and seizures |
| 5p15.3p15.2 | Cri-du-chat syndrome |
| 5q14.3q15 (MEF2C) | 5q14.2q15 deletion syndrome |
| 6p21.32 (SYNGAP1) | Neurodevelopmental disorder/autism spectrum |
| | disorder |
| 6q25.2q25.3 | 6q25.2q25.3 deletion syndrome |
| 7q11.2 | Neurodevelopmental disorder, autism spectrum |
| (AUTS2/KIAA0442) | disorder, and seizures |
| 7q11.23 | Williams syndrome or 7q11.23 duplication |
| | syndrome |
| 7q35 (CNTNAP2) | Autism spectrum disorder |
| 7q36.2 (DPP6) | Autism spectrum disorder |
| 8p23.1 | 8p23.1 deletion syndrome |
| 8q11.23 | Autism spectrum disorder |
| 8q22.1 | 8q22.1 deletion syndrome |
| 8q24.11q24.13 | Langer-Giedion syndrome |
| 9q22.3 | 9q22.3 deletion syndrome |
| 9q34.3 (EHMT1) | Kleefstra syndrome (9q subtelomeric deletion |
| | syndrome) |
| 10p15.3 | Neurodevelopmental disorder |
| 10p14p13 | DiGeorge syndrome 2 (Velocardiofacial |
| | syndrome 2) |
| 10q22.3q23.31 | 10q22.3q23.31 deletion syndrome |
| 11p13 | WAGR syndrome |
| 11p11.2 | Potocki-Shaffer syndrome |
| 11q13.2 (SHANK2) | Autism spectrum disorder |
| 11q23qter | Jacobsen syndrome |
| 12p | Mosaic tetrasomy 12p (Pallister-Killian |
| | syndrome) |
| 12q14 | 12q14 deletion syndrome |
| Chromosome 13 | Trisomy 13 (Patau syndrome) |
| 13q | 13q deletion syndrome (partial trisomy 13) |
| 14q23.2q23.3 | Intellectual disability and spherocytosis |
| Chromosome 15 | Tetrasomy 15/Inverted duplicated chromosome |
| | 15 (Isodicentric chromosome 15) syndrome |
| 15q11.2 (UBE3A) | Neurodevelopmental disorder/autism spectrum |
| | disorder/Angelman syndrome/Prader-Willi |
| | syndrome |
| 15q13.3 | 15q13.3 deletion or duplication syndrome |
| 15q24.1q24.2 | 15q24.1 deletion syndrome |
| 16p13.3 (A2BP1) | Neurodevelopmental disorder, autism spectrum |
| | disorder, and seizures |

TABLE B
X linked copy number variations

| Chromosomal | |
|---|---|
| Location | Associated condition/clinical features |
| X chromosome | Monosomy X (Turner syndrome)/Klinefelter |
| | syndrome/XXY syndrome |
| Xp22.32 (NLGN4X) | Autism spectrum disorder |
| Xp22.2 (OFD1) | Joubert syndrome/Orofacial digital syndrome/ |
| | Simpson-Golabi Bemhel syndrome |
| Xp22.13 (CDKL5) | CDKL5-related conditions |
| Xp22.2 (AP1S2) | XLID |
| Xp22.11 (PTCHD1) | Autism spectrum disorder |
| Xp22.1 (SMS) | Snyder-Robinson syndrome |
| Xp22 (RPS6KA3) | Coffin-Lowry syndrome |
| Xp21.3 (ARX) | X-linked intellectual disability (XLID) |
| Xp21.3p21.2 | XLID |
| (IL1RAPL1) | |
| Xp21.1 (OTC) | Ornithine transcarbamylase deficiency |
| Xp11.4 (CASK) | XLID and FG syndrome |
| Xp11.3 (ZNF674) | XLID |
| Xp11.23 (FTSJ1) | XLID |
| Xp11.23 (PQBP1) | XLID |
| Xp11.23 (SYN1) | XLID |
| Xp11.23 (ZNF81) | XLID |
| Xp11.22 (HUWE1) | XLID |
| Xp11.22 (SHROOM4) | XLID |
| Xp11.22p11.21 | Cornelia de Lange syndrome |
| (SMC1A) | |
| Xp11.2 (PHF8) | XLID |
| Xp11 (ZNF41) | XLID |
| Xp11 | XLID |
| (KDM5C/JARID1C) | |
| Xq11.1 (ARHGEF9) | XLID |
| Xq11.4 | XLID |
| (TSPAN7/TM4SF2) | |
| Xq12 (OPHN1) | XLID |
| Xq13 (DLG3) | XLID |
| Xq13.1 (NLGN3) | Autism spectrum disorder |
| Xq13.2 | Allan-Herndon-Dudley syndrome |
| (SLC16A2/MCT8) | |
| Xq21.1 (ATRX) | Alpha-thalassemia/X-linked intellectual |
| | disabilty syndrome |
| Xq22 | XLID |
| (ACSL4/FACL4) | |
| Xq22 (NXF5) | XLID |
| Xq22 (PLP1) | Pelizaeus-Merzbacher disease |
| Xq22.3 (DCX) | X-linked lissencephaly |
| Xq22.3 (PAK3) | XLID |
| Xq24 (CUL4B) | XLID |
| Xq24 (UPF3B) | XLID |
| Xq25 (GRIA3) | XLID |
| Xq25 (OCRL 1) | Occulocerebrorenal syndrome of Lowe |
| Xq25 (ZDHHC9) | XLID |
| Xq26.1 (HPRT1) | Lesch-Nyhan syndrome |
| Xq26.3 | X-linked Angelman-like syndrome |
| (NHE6/SLC9A6) | |
| Xq28 (ABCD1) | X-linked Adrenoleukodystrophy |
| Xq28 (GDI1) | XLID |
| Xq28 (MECP2) | Rett syndrome/MECP2-related conditions |
| Xq28 (RAB39B) | XLID |

Developmental delay disorders are an ever growing group of disorders. Many developmental delay disorders are associated with aberrant copy number (gain or loss of copy number) of a particular subchromasomal region and are known as microdeletion and microduplication syndromes. Various microdeletion and microduplication syndromes are disclosed in Weiss et al. (“Microdeletion and microduplication syndromes” J. of Histochemistry & Cytochemistry 60(5) 346; 2012, incorporated by reference in its entirety for all purposes). In one embodiment, the present invention provides a method and/or assay components (e.g., oligonucleotides that specifically hybridize to CNV regions) for the diagnosis of the microdeletion and/or microduplication syndromes disclosed in Weiss et al., and/or a method and/or assay components to select a patient for the treatment of such microdeletion and/or microduplication syndrome. Specifically, any chromosomal deletion or duplication that results in symptoms such as hypotonia (muscle weakness), intellectual disability, dysmorphic physical features, repetitive behaviors is included under the umbrella of developmental delay conditions that can be detected using the present invention. Specific examples include, but are not limited to, the disorders set forth in Tables A and B and specifically, ASD, chromosome 22q13.3 deletion syndrome, 22q11.2 deletion syndrome (DiGeorge syndrome), 1p36 deletion syndrome, Prader-Willi syndrome, Angelman syndrome, chromosome 1p36 deletion syndrome, Wolf-Hirschhorn Syndrome (also known as chromosome 4p-Syndrome), 1q21.1 duplication syndrome, and chromosome 15q duplication syndrome.

Childhood developmental delay disorders may also include, but are not limited to, Rett syndrome, Noonan/Costello/CFC syndromes, Tuberous sclerosis, ADHD, developmental delay (DD), Tourette syndrome, and Dyslexia. The OMIM web site (internet address can be found at ncbi.nlm.nih.gov/omim) keeps an updated list of disorders and a description of the specific genotype identified, that can be accessed by the skilled person.

The Diagnostic and Statistical Manual of Mental Disorders, Fourth Edition—Text Revision currently defines five disorders, sometimes called pervasive developmental disorders (PDDs), as ASD. These include: Autistic disorder (classic autism), Asperger's disorder (Asperger syndrome (AS)), Pervasive developmental disorder not otherwise specified (PDD-NOS), Rett's disorder (Rett syndrome), and Childhood disintegrative disorder (CDD). It is noted that the majority of Rett syndrome cases are known to be caused by mutations in either the MeCP2 gene or the CDKL5 gene and it is anticipated that updated revisions of the Diagnostic and Statistical Manual of Mental Disorders will classify Rett syndrome separately from ASD. Therefore, in certain embodiments, ASD does not include Rett syndrome. However, as provided in Table B, the present invention is useful for selecting a patient for the diagnosis of Rett syndrome and or selecting a patient for the treatment of Rett syndrome. Autism shall be understood as any condition of impaired social interaction and communication with restricted repetitive and stereotyped patterns of behavior, interests and activities present before the age of 3, to the extent that health may be impaired. AS is distinguished from autistic disorder by the lack of a clinically significant delay in language development in the presence of the impaired social interaction and restricted repetitive behaviors, interests, and activities that characterize ASD. PDD-NOS is used to categorize individuals who do not meet the strict criteria for autism but who come close, either by manifesting atypical autism or by nearly meeting the diagnostic criteria in two or three of the key areas.

In one aspect of the invention, the present invention provides a method of determining the presence or absence of a deletion or duplication syndrome in a subject. In one embodiment, the deletion or duplication syndrome is selected from one or more of the deletion or duplication syndromes set forth at Table A and/or Table B. In a further embodiment, the subject is selected for therapy of the deletion or duplication syndrome if the CNV is present, and is at least about 500 bases in length.

The method in one embodiment comprises probing a sample obtained from the subject for the presence or absence of one or more copy number variants (CNVs) associated with the chromosomal deletion or duplication syndrome, and if the CNV is present, optionally analyzing the size of the deletion or duplication of at least one CNV. In one embodiment, the probing step comprises mixing the sample with five or more oligonucleotides that are substantially complementary to portions of the genomic DNA sequence associated with the deletion or duplication syndrome under conditions suitable for hybridization of the five or more oligonucleotides to their complements or substantial complements; detecting whether hybridization occurs between the five or more oligonucleotides to their complements or substantial complements, or a subset thereof and obtaining hybridization values of the sample based on the detecting step.

The determination of whether the CNV is present or absent, in one embodiment, comprises comparing the hybridization values of the sample to reference hybridization value(s) from at least one training set comprising hybridization value(s) from a sample that is positive for the one or more CNVs, or hybridization value(s) from a sample that is negative for the one or more CNVs. In one embodiment, the comparing step comprises determining a correlation between the hybridization values obtained from the sample and the hybridization value(s) from the at least one training set (which may be included in a database of values or a sample training set). A determination is then made regarding the presence or absence of the at least one CNV followed by an assessment of whether the subject has the chromosomal deletion or duplication syndrome.

In one embodiment, the sample comprises restriction digested double stranded DNA obtained from genomic DNA fragments; restriction digested single stranded DNA obtained from genomic DNA fragments; amplified restriction digested genomic DNA single stranded fragments; amplified restriction digested genomic DNA double stranded fragments; or a combination thereof. In a further embodiment, the sample is free of histone proteins. In even a further embodiment, the amplified restriction digested genomic DNA single stranded fragments comprise a detectable label chemically attached to individual single stranded fragments. In yet a further embodiment, the amplified restriction digested genomic DNA single stranded fragments further comprise adapter sequences. In one embodiment, the adapter sequences are introduced via adapter-specific primers.

The present invention also provides methods for selecting a subject for a treatment or predicting the response of a subject to a treatment for a childhood development disorder and specifically a duplication or deletion syndrome (e.g., a duplication or deletion syndrome affecting gene associated with mitochondrial function). Treatments for a childhood development disorder encompassed by the methods provided herein include both pharmacological treatments and behavioral treatments. For example, if the CNV is present and the size of the duplication or deletion is greater than or equal to about 500 bp, the subject is diagnosed with the deletion or duplication syndrome and/or is selected for treatment of the syndrome. Alternatively or additionally, if the CNV is present and the size of the duplication or deletion is greater than or equal to about 500 bp, it is predicted that the subject will respond to treatment of the deletion or duplication syndrome, for example, treatment of a clinical manifestation of the deletion or duplication syndrome (e.g., a clinical manifestation of WHS).

The at least one CNV, in one embodiment, is detected using a nucleic acid hybridization assay, for example a genomic DNA hybridization assay with oligonucleotides specific for the at least one CNV. The nucleic acid hybridization assay selected from a PCR based assay, a NanoString assay (e.g., nCounter CNV Analysis) or a sequencing assay (for example high throughput sequencing, single molecule sequencing, next-generation sequencing, etc.), or a combination thereof.

In another embodiment, the one or more CNVs is associated with one or more mitochondrial associated genes, for example, one or more of the genes set forth in Table 15, herein. Accordingly, the present invention provides methods for determining the presence or absence of a mitochondrial related disorder, and methods for predicting the likelihood of whether a subject will develop such a disorder, e.g., by probing for one or more CNVs that affect mitochondrial associated genes.

In another embodiment, a method for selecting a subject for mitochondrial therapy is provided. In one embodiment, the method comprises probing a genetic sample from the subject for the presence or absence of at least one copy number variant (CNV) associated with a mitochondrial gene, for example a gene set forth in Table 15. In one embodiment, the probing step comprises mixing the sample with five or more oligonucleotides that are substantially complementary to portions of the genomic DNA sequence associated with the deletion or duplication syndrome under conditions suitable for hybridization of the five or more oligonucleotides to their complements or substantial complements; detecting whether hybridization occurs between the five or more oligonucleotides to their complements or substantial complements, or a subset thereof and obtaining hybridization values of the sample based on the detecting step. The determination of whether the CNV is present or absent, in one embodiment, comprises comparing the hybridization values of the sample to reference hybridization value(s) from at least one training set comprising hybridization value(s) from a sample that is positive for the one or more CNVs, or hybridization value(s) from a sample that is negative for the one or more CNVs. In one embodiment, the comparing step comprises determining a correlation between the hybridization values obtained from the sample and the hybridization value(s) from the at least one training set (which may be included in a database of values or a sample training set). A determination is then made regarding the presence or absence of the at least one CNV followed by an assessment of whether the subject has the chromosomal deletion or duplication syndrome.

In a further embodiment, if the CNV genetic marker is detected, the subject is selected for mitochondrial therapy and is administered mitochondrial therapy. Categories of mitochondrial functions are instructive as to the type of therapy to employ. For example, categories of mitochondrial function include but are not limited to, NADH dehydrogenase ubiquinone, ATP5 (F1 Complex), cytochrome c reductase, mitochondrial solute/metabolite carriers, mitochondrial ATPases, thioredoxin, ribosomal complex proteins, creatinine kinases, glutathione S transferase family proteins, mitochondrial nucleotidase, OXPHOS proteins, ATP Binding Cassette (ABC) transporters, humanin family of mitochondrial peptides, and pathways or processes such as electron transport, regulation of oxidative stress, apoptosis, fatty acid synthesis, heme biosynthesis, mitochondrial maintenance, and immune responses. In one embodiment, the type of mitochondrial therapy selected for the subject is dependent on the type of function associated with the one or more mitochondrial genes having one or more CNV. The mitochondrial therapy, in one embodiment, is selected from an antioxidant, oxygen, arginine, Coenzyme Q10, idebenone, benzoquinone therapeutics (e.g., alpha-tocotrienol quinone (EPI-743) (Edison Pharmaceuticals)), creatine, lipoic acid, dichloroacetate (DCA), citrulline, or a combination thereof. In a further embodiment, if the patient is selected for mitochondrial therapy based on the results of the CNV analysis, the method comprises treating the subject with quinone (EPI-743) (Edison Pharmaceutical's).

In one embodiment, the method for selecting a subject for a deletion or duplication syndrome therapy or for predicting the response of a subject to a deletion or duplication syndrome therapy comprises detecting the presence or absence in the genetic sample from the subject the presence of 1, 2, 10, 15, 20, 25, 30, 40, 50, 60, 70, 80, 90, 100, 150, 200, or more CNVs.

In one embodiment, the present invention provides a method for selecting a subject for a mitochondrial therapy. In a further embodiment, the subject has previously been diagnosed with one or more disorders, a developmental delay disorder. In a further embodiment, the development disorder is characterized as an ASD. In one embodiment, the method comprises detecting in a genetic sample from the subject the presence or absence of at least one CNV, wherein the at least one CNV is of one or more mitochondrial associated genes, and selecting the subject for mitochondrial therapy if the at least one CNV is detected. In one embodiment, the method comprises detecting in the genetic sample from the subject, the presence of from 1 to 100, from 2 to 75, from 5 to 50, or from 10 to 25 CNVs of one or more mitochondrial disease-associated genes. In one embodiment, the method comprises selecting the subject for mitochondrial therapy if the presence of at least 2, at least 5, at least 10, at least 25, or at least 50 of the CNVs are detected. In one embodiment, the least one CNV is detected using one or more sets of oligonucleotides. In one embodiment, the one or more sets of oligonucleotides are present on an array, such as a high density microarray or are used in an alternative hybridization assay such as a NanoString or genomic sequencing assay.

The methods provided herein are useful for determining whether a subject has a deletion or duplication syndrome associated with developmental delay, for example one or more of the disorders set forth in Table A and/or Table B. In one embodiment of this aspect, the method comprises selecting the subject for treatment of the deletion or duplication syndrome, for example treatment of a clinical manifestation of the deletion or duplication syndrome. In one embodiment, the method comprises detecting in a genetic sample from the subject the presence of at least one copy number variant (CNV) associated with the deletion or duplication syndrome, analyzing the size of the deletion or duplication, and determining that the patient has the deletion or duplication syndrome if the size of the deletion or duplication is at least about 500 bp, at least about 1,000 bp, at least about 10,000 bp, at least about 100,000 bp, at least about 1 mega base pairs (Mb), at least about 5 Mb, at least about 10 Mb, at least about 15 Mb, at least about 20 Mb, or at least about 50 Mb. CNVs and their respective size are detected by nucleic acid hybridization assays with primers (oligonucleotides) that specifically hybridize to the chromosomal DNA of interest, as explained below (see, e.g., the sequence listing for probes amenable for use with the present invention).

Similarly, the subject is identified as at risk for a clinical manifestation of the deletion or duplication syndrome (and accordingly, selected for treatment for the deletion or duplication syndrome) if the size of the deletion or duplication is at least about 500 bp, at least about 1,000 bp, at least about 10,000 bp, at least about 100,000 bp, at least about 1 mega base pairs (Mb), at least about 5 Mb, at least about 10 Mb, at least about 15 Mb, at least about 20 Mb, or at least about 50 Mb. In another embodiment, the subject is identified as at risk for a clinical manifestation of the deletion or duplication syndrome (and accordingly, selected for treatment for the deletion or duplication syndrome) if the size of the deletion or duplication is about 500 bp to about 20 Mb, or about 500 bp to about 10 Mb, or about 500 bp to about 5 Mb, or about 500 bp to about 1 Mb, or about 500 bp to about 500,000 bp, or about 500 bp to about 100,000 bp, or about 500 bp to about 50,000 bp.

Determination of the presence or absence of the deletion or duplication syndrome, and accordingly, selection for treatment of the deletion or duplication syndrome is dependent upon where the at least one CNV occurs in the genome. Tables A and B provide various deletion and duplication syndromes and corresponding chromosomal regions where CNVs are known to occur in patients having the respective disorder. Therefore, the CNV location can be mapped to a disorder for diagnosis and further identification of the patient for treatment of the disorder (i.e., selection of the patient for treatment).

Besides the syndromes set forth in Tables A and B, exemplary deletion syndromes that can be diagnosed with the methods and compositions provided herein include but are not limited to, for example, Wolf-Hirschhorn (4p) syndrome (WHS), 22q11.2 deletion syndrome (DiGeorge syndrome), and 1p36 deletion syndrome. Exemplary duplication syndromes include, for example, 1q21.1 duplication syndrome or chromosome 15q duplication syndrome. Exemplary clinical manifestations of such disorders include, for example, congenital heart disease, seizure, renal disease, intellectual disability, developmental delay, vision loss, blindness, or other condition affecting ears, skin, teeth, or skeletal development; or a combination thereof. Once a deletion or duplication CNV is identified in a respective subject, the patient in one embodiment is selected for treatment of one or more of the clinical manifestations provided above.

One clinical manifestation that a patient, for example a WHS patient, can be selected for treatment for, is status epilepticus. Accordingly, in one embodiment, the present invention provides a method for selecting a subject for treatment of status epilepticus. Status epilepticus is a life-threatening seizure disorder in which seizures are persistently present in the brain. In one embodiment, the subject in need of treatment for status epilepticus has an additional deletion or duplication syndrome. In one embodiment, the method comprises detecting in a genetic sample from the subject the presence of a CNV associated with a deletion or duplication syndrome. In a further embodiment, the method further comprises detecting in the genetic sample a second CNV provided in Table 3 or Table 4. The present invention also provides a method for selecting a patient for therapy with a glutamatergic or GABAergic drug. Such drugs are known in the art and include glutamate receptor or GABA agonists, antagonists, or allosteric modulators.

In one embodiment, the methods of the present invention comprise detecting in a genetic sample from a subject the presence of at least one CNV. In a further embodiment, the methods provided herein comprise detecting in the genetic sample from the subject the presence of 2, 10, 15, 20, 25, 30, 40, 50, 60, 70, 80, 90, 100, 150, 200, or more CNVs. In another embodiment, the methods comprise detecting in the genetic sample from the subject the presence of from 1 to 100, from 2 to 75, from 5 to 50, or from 10 to 25 CNVs. In one embodiment, the methods provided herein comprise selecting a subject for treatment with a therapy or for treatment for a particular disease, disorder, or condition if the presence of at least 2, at least 5, at least 10, at least 25, or at least 50 CNVs are detected. In some embodiments, the least one CNV is detected using one or more sets of oligonucleotides. In one embodiment, the one or more sets of oligonucleotides are present on an array, such as a high density microarray.

As used herein, the term “ICD-9” refers to the International Classification of Diseases, 9^{th} Revision. This set of classifications is available on the Centers for Disease Control and Prevention website and provides a standardized format for reporting disease classification and mortality statistics.

As used herein, the term “subject” refers to a vertebrate, for example, a mammal. Thus, the subject can be a human. The term does not denote a particular age or sex. Thus, adult and newborn subjects, as well as fetuses, whether male or female, are intended to be covered. Unless otherwise specified, the term “patient” includes human and veterinary subjects.

A “copy number variant” (CNV) includes copy number duplications and deletions, and encompasses a copy number change involving a DNA fragment that is about 500 bp or larger (see e.g., Feuk, et al., 2006 Nature Reviews Genetics, 7, 85-97, incorporated by reference in its entirety herein for all purposes). CNVs described herein do not include those variants that arise from the insertion/deletion of transposable elements (e.g., .about.6-kb Kpnl repeats) to minimize the complexity of CNV analyses. The term CNV therefore encompasses previously introduced terms such as large-scale copy number variants (LCVs; lafrate et al. 2004 Nat Genet. 36:949-951, incorporated by reference in its entirety herein for all purposes), copy number polymorphisms (CNPs; Sebat et al. 2004 Science. 305:525-528, incorporated by reference in its entirety herein for all purposes), and intermediate-sized variants (ISVs; Tuzun et al. 2005 Nat Genet. 37:727-732, incorporated by reference in its entirety herein for all purposes), but not retroposon insertions.

With respect to single stranded nucleic acids, particularly oligonucleotides, the term “specifically hybridize” refers to the association between two single-stranded nucleotide molecules of sufficient complementary sequence to permit such hybridization under pre-determined conditions generally used in the art. In particular, in one embodiment the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence. For example, specific hybridization can refer to a sequence which hybridizes to a first chromosomal region but does not specifically hybridize to a second chromosomal region. Appropriate conditions enabling specific hybridization of single stranded nucleic acid molecules of varying complementarity are well known in the art.

A CNV genetic marker refers to a genomic DNA sequence having a copy number variation, with a known location on a chromosome, which can be used to diagnose subjects with a duplication or deletion syndrome, for example a duplication or deletion syndrome associated with developmental delay and/or to select a subject for treatment of such a syndrome.

The CNV genetic markers associated with ASD described herein, were identified in an extensive replication/refinement study of CNV markers. In particular, a custom array was designed and used to genotype about 3000 individuals with autism and 6000 individuals with normal development. A combination of 2 different statistical and bioinformatics algorithms was used to make the CNV calls and proved to be highly accurate. In particular, 97% of the CNVs called using the combination of algorithms were subsequently validated by other laboratory methods, as compared to 30% using only the individual algorithms (see Example 1). The CNV genetic markers associated with ASD identified herein are provided in Tables 3 and 4. The CNV genetic markers shown in Tables 3 and 4 are those CNV genetic markers having an odds ratio (the likelihood that a given genetic marker is relevant to a diagnosis of ASD in an individual) of 2 or higher.

While certain of the CNV genetic markers associated with developmental delay shown in Table 4 overlap with previously identified CNV genetic markers, the CNVs had not been previously extensively refined and validated until the present study. Therefore, the present invention provides newly identified CNV genetic markers as well as refined and validated genetic markers, that greatly improve the diagnostic yield of developmental delay diagnostic tests over what was previously known. Thus, the present disclosure provides a more diagnostically comprehensive and accurate set of CNV genetic markers associated with developmental delay that can be used in the diagnosis of deletion and/or duplication syndromes associated with developmental delay. Illustrative DNA probes that can be used to genotype individuals for the presence of CNVs associated with developmental delay syndromes, e.g., ASD, are provided in the sequence listing which includes SEQ ID NOs:1-83,433. These DNA probes also include custom probes to genotype other childhood developmental delay disorders, including for example, Rett syndrome, Noonan/Costello/CFC syndromes, Tuberous sclerosis, ADHD, DD, and Tourette syndrome. Illustrative DNA probes for detecting the presence of CNVs associated with developmental delay are provided in SEQ ID NOs: 7410-7426; 12508-12563; 27988-28001; 31283-31314; 32494-32587; 33402-39860; 51803-52100; 61165-61290; 62966-62998; 64149-64167; 69319-69561.

The CNV genetic markers associated with the diagnosis of deletion and/or duplication syndromes associated with developmental delay as described herein are generally defined by their chromosomal location and are referred to by the most recent human genome coordinates (e.g., hg19 chromosomal location coordinates). However, as would be understood by the skilled artisan, as the exact region of the CNV (e.g., the region of highest significance) is further characterized and refined, the CNV region boundaries may shift to the left or to the right while getting smaller, or may get smaller within the same region as originally defined. For example, the CNVs listed in Table 3 are referred to by the CNV region as defined in the discovery cohort as well as the CNV region as defined in the replication cohort. As shown in Table 3, the CNV region for the first listed marker has been reduced from the region spanning chr1:145714421-146101228 to the region spanning chr1: 145703115-145736438, with the left boundary shifting further to the left. The region boundaries for CNV marker number 6 listed in Table 3 have shifted to the right and have been reduced. Therefore, as would be understood by the skilled person, the CNV markers associated with ASD as described herein comprise the CNV region as described herein and include the surrounding region to the left and to the right of the CNV chromosomal region as described herein. Thus, in certain embodiments, the chromosomal region encompassing the CNV genetic markers associated with one of the duplication or deletion syndromes described herein may comprise the chromosomal region 50, 100, 200, 300, 400, 500, 600, 700, 800, 900, 1000, 1500, 2000, 2500, 3000, 3500, 4000, 4500, 5000, 6000, 7000, 8000, 9000, 10000, 15,000, 20000, 30000, 40000, 50,000, 60,000, 70,000, 80,000, 90,000, 100,000, or more positions to the left and/or to the right of the chromosomal region as described herein.

In one embodiment, reagents for detecting the CNV genetic markers as described herein include reagents which specifically hybridize to the chromosomal regions surrounding the region specifically described herein. In particular, a nucleic acid reagent for detecting the CNV genetic markers as described herein may specifically hybridize to the chromosomal region 50, 900, 1000, 1500, 2000, 2500, 3000, 3500, 4000, 4500, 5000, 6000, 7000, 8000, 9000, 10000, 15,000, 20000, 30000, 40000, 50,000, 60,000, 70,000, 80,000, 90,000, 100,000, or more positions to the left and/or to the right of the chromosomal region of the CNV genetic marker as described herein.

In embodiments where methods are provided for diagnosis of subjects with a deletion or duplication syndrome associated with mitochondrial function, the CNV that is probed for is a copy number variant of one or more of the genes set forth in Table 18, i.e., a gene associated with mitochondrial function. For example, in one embodiment, the CNV is a CNV that affects one or more, two or more, five or more or ten or more of the mitochondrial associated genes set forth in Table 15. In another embodiment, the at least one CNV is a CNV that affects one to ten, one to nine, one to eight or one to five of the mitochondrial associated genes set forth in Table 18.

In one embodiment, the presence of one or more CNVs described herein indicates that an individual is affected with the deletion or duplication syndrome, or is predisposed to developing the deletion or duplication syndrome. In another embodiment, the presence of one or more CNV genetic markers described herein may be predictive of whether an individual is at risk for or susceptible to the deletion or duplication syndrome. If certain genetic polymorphisms (e.g., CNVs) are detected more frequently in people with the deletion or duplication syndrome, the variations are said to be “associated” with the particular deletion or duplication syndrome. In this regard, variations may be associated with any of the deletion or duplication syndromes set forth herein, for example the deletion or duplication syndromes set forth in Table A and Table B. The polymorphisms associated with ASD may either directly cause the disease phenotype or they may be in linkage disequilibrium (LD) with nearby genetic mutations that influence the individual variation in the disease phenotype. As used herein, LD is the nonrandom association of alleles at 2 or more loci.

In each of the methods described herein, the presence or absence of one or more CNVs (e.g., one or more, two or more, five or more, ten or more CNVs) is probed for in a sample obtained from a subject. “Sample” or “biological sample,” as used herein, refers to a sample obtained from a human subject or a patient, which may be tested for a particular molecule, for example one or more of the CNVs associated with a deletion or duplication syndrome, as set forth herein. Samples may include but are not limited to cells, buccal swab sample, body fluids, including blood, serum, plasma, urine, saliva, cerebral spinal fluid, tears, pleural fluid and the like. Samples that are suitable for use in the methods described herein contain genetic material, e.g., genomic DNA (gDNA). Non-limiting examples of sources of samples include urine, blood, and tissue. The sample itself will typically consist of nucleated cells (e.g., blood or buccal cells), tissue, etc., removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., genomic DNA. All samples obtained from a subject, including those subjected to any sort of further processing, are considered to be obtained from the subject.

The sample in one embodiment, is further processed before the detection of the presence or absence of the one or more CNVs. For example, DNA, e.g., genomic DNA in a cell or tissue sample can be separated from other components of the sample. The sample can be concentrated and/or purified to isolate genomic DNA in a non-natural state. Specifically, genomic DNA exists as genomic chromosomal DNA and is a tightly coiled structure, wherein the DNA is coiled many times around histone proteins that support the genomic DNA and chromosomal structure. In the methods provided herein, the higher order structure of the genomic DNA (e.g., tertiary and quaternary structures) is modified considerably by eliminating histone proteins from the sample, and digesting the genomic DNA into fragments with frequent cutting restriction endonucleases. Genomic DNA therefore does not exist as natural genomic DNA, it is present in small fragments (with lengths ranging from about 100 basepairs to about 500 basepairs) rather than as large polymers on individual chromosomes, comprising tens to hundreds of megabase pairs.

Once the genomic DNA is digested and chemically modified into a non-natural sequence and structure, it is amplified, in one embodiment, with primers that introduce an additional DNA sequence (adapter sequence) onto the fragments (with the use of adapter-specific primers). Amplification therefore serves to create non-natural double stranded molecules, by introducing adapter sequences into the already non-natural restriction digested, and chemically modified genomic DNA. Further, as known to those of ordinary skill in the art, amplification procedures have error rates associated with them. Therefore, amplification introduces further modifications into the smaller DNA fragments. In one embodiment, during amplification with the adapter-specific primers, a detectable label, e.g., a fluorophore, is added to single strand DNA fragments. Amplification therefore also serves to create DNA complexes that do not occur in nature, at least because of (i) the addition of adapter sequences, (ii) the error rate associated with amplification, (iii) the disparate structure of these complexes as compared to what exists in nature, i.e., large polymers of DNA wrapped around histone proteins and the chemical addition of a detectable label to the DNA fragments.

Once a sample is obtained, it is interrogated for one or more of the CNVs set forth herein.

In general, the one or more CNVs can be identified using a nucleic acid hybridization assay alone or in combination with an amplification assay, i.e., to amplify the nucleic acid in the sample prior to detection. In one embodiment, the genomic DNA of the sample is sequenced or hybridized to an array, as described in detail herein. A determination is then made as to whether the sample includes the one or more CNVs depending on the detected hybridization pattern, or rather, includes the “normal” or “wild type” sequence (also referred to as a “reference sequence” or “reference allele”).

Detection using a hybridization assay comprises the generation of non-natural DNA complexes, that is, DNA complexes that do not exist in nature. As mentioned above, the DNA that is used in the hybridization assay is already in a non-natural state because of various modifications, specifically, (i) modifications to the length of the DNA, (ii) modifications to the primary structure of the DNA via the addition of adapter sequences during the amplification process, (iii) modifications to the higher order structure of the DNA due to the elimination of histone proteins and other cellular material, (iv) chemical modifications due to the addition of a detectable label to the digested DNA fragments, and (v) further chemical modifications due to introduction of bases that do not occur in the native chromosomal DNA, due to inherent error in the amplification reaction (leading to further change in primary structure as compared to chromosomal genomic DNA).

In the case of a hybridization assay, for example a microarray assay or bead based assay, hybridization occurs between the non-natural fragments described above and an immobilized sequence of known identity. Therefore, the product of the hybridization assay is further removed from DNA duplexes that exist in nature, because of the reasons set forth above, and because each is immobilized, for example to a glass slide or bead.

In one embodiment, if the hybridization assay reveals a difference between the sequenced region and the reference sequence (which can be included in the hybridization assay as a control, or in a dataset, for example, a statistical training set), a CNV has been identified. Certain statistical algorithms can aid in this determination, as described herein. The fact that a difference in nucleotide sequence is identified at a particular site that determines that a CNV exists at that site.

For example, an oligonucleotide or oligonucleotide pair can be used in the methods described herein, for example in a microarray or polymerase chain reaction assay, to detect the one or more CNVs.

The term “oligonucleotide” refers to a relatively short polynucleotide (e.g., 100, 50, 20 or fewer nucleotides) including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms. Oligonucleotides for use in detecting the presence or absence of certain CNVs associated with chromosomal deletion or duplication syndromes are provided in the accompanying sequence listing.

In the context of the present invention, an “isolated” or “purified” nucleic acid molecule, e.g., a DNA molecule or RNA molecule, is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. In another embodiment, the “isolated nucleic acid” comprises a DNA molecule inserted into a vector, such as a plasmid or virus vector, or integrated into the genomic DNA of a prokaryote or eukaryote. An “isolated nucleic acid molecule” may also comprise a cDNA molecule or an oligonucleotide primer or probe, or additional sequences added onto a fragment of DNA, for example, an adapter sequence added to a restriction cut portion of genomic DNA.

As used herein a set of oligonucleotides, in one embodiment, comprises from about 2 to about 100 oligonucleotides, all of which specifically hybridize to a particular CNV or region thereof, which includes for example one of the chromosomal regions set forth in Table A or Table B, or one or more of the CNVs set forth herein. In one embodiment, a set of oligonucleotides comprises from about 5 to about 100 oligonucleotides (or from about 5 to about 30 oligonucleotide pairs), from about 10 to about 100 oligonucleotides (or from about 10 to about 100 oligonucleotide pairs), from about 10 to about 75 oligonucleotides (or from about 10 to about 75 oligonucleotide pairs), from about 10 to about 50 oligonucleotides (or from about 10 to about 0 oligonucleotide pairs). In one embodiment, a set of oliognucleotides comprises about 15 to about 50 oligonucleotides, all of which specifically hybridize to a particular CNV associated with a deletion or duplication syndrome, for example, a deletion or duplication syndrome associated with developmental delay. In one embodiment, a set of oligonucleotides comprises DNA probes, e.g., genomic DNA probes. In one embodiment, the DNA probes comprise DNA probes that overlap in genomic sequence. In another embodiment, the DNA probes comprise DNA probes that do not overlap in genomic sequence. In one embodiment, the DNA probes provide detection coverage over the length of a CNV associated with a deletion or duplication syndrome, for example, a deletion or duplication syndrome associated with developmental delay. In another embodiment, a set of oligonucleotides comprises amplification primers that amplify a CNV or region thereof, wherein the CNV is associated with a deletion or duplication syndrome, for example, a deletion or duplication syndrome associated with developmental delay. In this regard, sets of oligonucleotides comprising amplification primers may comprise multiplex amplification primers. In another embodiment, the sets of oligonucleotides or DNA probes may be provided on an array, such as solid phase arrays, chromosomal/DNA microarrays, or micro-bead arrays.

Illustrative reagents for detecting genetic markers include nucleic acids, and in particular include oligonucleotides. A nucleic acid can be DNA or RNA, and may be single or double stranded. In one embodiment, the oligonucleotides are DNA probes, or primers for amplifying nucleic acids of genetic markers. In one embodiment, the oligonucleotides of the present invention are capable of specifically hybridizing (e.g, under stringent hybridization conditions), with complementary regions of a genetic marker associated with ASD containing a genetic polymorphism described herein, such as a dopy number variation. Oligonucleotides can be naturally occurring or synthetic, but are typically prepared by synthetic means. Oligonucleotides, as described herein, may include segments of DNA, or their complements. The exact size of the oligonucleotide will depend on various factors and on the particular application and use of the oligonucleotide. Oligonucleotides, which include probes and primers, can be any length from 3 nucleotides to the full length of a target nucleic acid molecule of interest (e.g., a nucleic acid molecule of a CNV genetic marker associated with a deletion or duplication syndrome set forth herein, such as those provided in Tables A and B), and explicitly include every possible number of contiguous nucleic acids from 3 through the full length of a target polynucleotide of interest. Thus, oligonucleotides can be between 5 and 100 contiguous bases, and often range from 5, 10, 12, 13, 14, 15, 16, 17, 18, 19, 20, 21, 22, 23, 24, or 25 nucleotides to 30, 35, 40, 45, 50, 55, 60, 65, 70, 75, 80, 85, 90, 95, or 100 nucleotides. Oligonucleotides between 5-10, 5-20, 10-20, 12-30, 15-30, 10-50, 20-50 or 20-100 bases in length are common.

Oligonucleotides of the present invention can be RNA, DNA, or derivatives of either. The minimum size of such oligonucleotides is the size required for formation of a stable hybrid between an oligonucleotide and a complementary sequence on a nucleic acid molecule of the present invention (i.e., the copy number variant genetic markers described herein). The present invention includes oligonucleotides that can be used as, for example, probes to identify nucleic acid molecules (e.g., DNA probes) or primers to amplify nucleic acid molecules.

In one embodiment, an oligonucleotide may be a probe which refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. In certain embodiments, a probe can be between 5 and 100 contiguous bases, and is generally about 5, 10, 12, 13, 14, 15, 16, 17, 18, 19, 20, 21, 22, 23, 24, or 25 nucleotides in length, or may be about 30, 35, 40, 45, 50, 55, 60, 65, 70, 75, 80, 85, 90, 95, or 100 nucleotides in length. The probes herein are selected to be complementary to different strands of a particular target nucleic acid sequence. This means that the probes must be sufficiently complementary so as to be able to specifically hybridize or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. Illustrative probes for detecting the genetic markers associated with ASD and other childhood developmental delay disorders are set forth in SEQ ID NOs:1-83,443. In particular, DNA probes for detecting CNVs associated with ASD are set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561. (See also Table 11 for a description of the childhood developmental delay disorders and the custom DNA probes provided in the sequence listing and Table 14 from U.S. Provisional Application 61/977,462 and Table 14 from International PCT Publication No. 2014/055915, the disclosure of each of which is incorporated by reference in their entireties). As would be recognized by the skilled person, a specific probe or probe set disclosed herein for detecting a particular CNV associated with ASD (or other disorder), can be identified by using the hg19 chromosomal location start and end coordinates of a CNV of interest (e.g., a CNV listed in Table 3 or 4) to query Table 14 from the aforementioned references, to find a corresponding overlapping chromosomal location

In one embodiment, an oligonucleotide may be a primer, which refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in certain applications, an oligonucleotide primer is about 15-25 or more nucleotides in length, but may in certain embodiments be between 5 and 100 contiguous bases, and often be about 5, 10, 12, 13, 14, 15, 16, 17, 18, 19, 20, 21, 22, 23, 24, or 25 nucleotides long or, in certain embodiments, may be about 30, 35, 40, 45, 50, 55, 60, 65, 70, 75, 80, 85, 90, 95, or 100 nucleotides in length for. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

In one embodiment, detection of one or more CN Vs comprises the use of one or more DNA probes or sets of probes as set forth in SEQ ID NOs:1-83,443. In one embodiment, an array comprises at least 1, 2, 3, 4, 5, 6, 7, 8, 9, 10, 11, 12, 13, 14, 15, 16, 17, 18, 19, 20, or more DNA probes as set forth in SEQ ID NOs:1-83,443. In another embodiment, an array for identifying the genotype of a subject suspected of having ASD or other childhood developmental delay disorder, comprises at least about 25-2500, or at least 100, 1000, 10000, 15000, 16000, 17000, 18000, 19000, 20000, 25000, 30000, 35000, 40000, 45000, 50000, 55000, 60000, 65000 or more of the DNA probes forth in SEQ ID NOs:1-83,443. In another embodiment, an array for genotyping an individual for the presence of a CNV associated with ASD or other childhood developmental delay disorder, comprises the DNA probes set forth in the sequence listing and identified in Table 14 from U.S. Provisional Application 61/977,462 and Table 14 from International PCT Publication No. 2014/055915, the disclosure of each of which is incorporated by reference in their entireties that are custom probes for the CNVs listed in Tables 8 and 9, which specifically hybridize to the CNVs identified in Table 3 and 4. In one embodiment, an array for genotyping an individual for the presence of a CNV associated with ASD, comprises the DNA probes set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561.

In one embodiment, hybridization on a microarray is used to detect the presence of one or more SNPs in a patient's sample. The term “microarray” refers to an ordered arrangement of hybridizable array elements, e.g., polynucleotide probes, on a substrate.

In another embodiment of the invention, constant denaturant capillary electrophoresis (CDCE) can be combined with high-fidelity PCR (HiFi-PCR) to detect the presence of one or more CNVs. In another embodiment, high-fidelity PCR is used. In yet another embodiment, denaturing HPLC, denaturing capillary electrophoresis, cycling temperature capillary electrophoresis, allele-specific PCRs, quantitative real time PCR approaches such as TaqMan® is employed to detect the one or more CNVs. Other approaches to detect the presence of one or more CNVs, and in some cases, the size (i.e., as reported in bases or base pairs) of the one or more CNVs, amenable for use with the present invention include polony sequencing approaches, microarray approaches, mass spectrometry, high-throughput sequencing approaches, e.g., at a single molecule level, and the NanoString approach.

Hybridization detection methods are based on the formation of specific hybrids between complementary nucleic acid sequences that serve to detect nucleic acid sequence mutation(s) and are amenable for use with the methods described herein. Methods of nucleic acid analysis to detect polymorphisms and/or polymorphic variants (copy number variants) include, e.g., microarray analysis and real time PCR. Hybridization methods, such as Southern analysis, Northern analysis, or in situ hybridizations, can also be used (see Current Protocols in Molecular Biology, Ausubel et al., eds., John Wiley & Sons 2003, incorporated by reference in its entirety).

Other methods for use with the methods provided herein include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al. U.S. Pat. No. 5,288,644, each incorporated by reference in its entirety for all purposes); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989), incorporated by reference in its entirety), restriction enzyme analysis (Flavell et al., Cell 15:25 (1978); Geever et al., Proc. Natl. Acad. Sci. USA 78:5081 (1981), incorporated by reference in its entirety); quantitative real-time PCR (Raca et al., Genet Test 8(4):387-94 (2004), incorporated by reference in its entirety); heteroduplex analysis; chemical mismatch cleavage (CMC) (Cotton et al., Proc. Natl. Acad. Sci. USA 85:4397-4401 (1985), incorporated by reference in its entirety); RNase protection assays (Myers et al., Science 230:1242 (1985), incorporated by reference in its entirety); use of polypeptides that recognize nucleotide mismatches, e.g., E. coli mutS protein; allele-specific PCR, for example. See, e.g., U.S. Patent Publication No. 2004/0014095, which is incorporated herein by reference in its entirety.

In order to detect the CNV(s) described herein, in one embodiment, genomic DNA (gDNA) or a portion thereof containing the polymorphic site, present in the sample obtained from the subject, is first amplified. Such regions can be amplified and isolated by PCR using oligonucleotide primers designed based on genomic and/or cDNA sequences that flank the site. See e.g., PCR Primer: A Laboratory Manual, Dieffenbach and Dveksler, (Eds.); McPherson et al., PCR Basics: From Background to Bench (Springer Verlag, 2000, incorporated by reference in its entirety); Mattila et al., Nucleic Acids Res., 19:4967 (1991), incorporated by reference in its entirety; Eckert et al., PCR Methods and Applications, 1:17 (1991), incorporated by reference in its entirety; PCR (eds. McPherson et al., IRL Press, Oxford), incorporated by reference in its entirety; and U.S. Pat. No. 4,683,202, incorporated by reference in its entirety. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Natl. Acad. Sci. USA, 87:1874 (1990)), incorporated by reference in its entirety, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety. A variety of computer programs for designing primers are available.

In one example, a sample (e.g., a sample comprising genomic DNA), is obtained from a subject. The DNA in the sample is then examined to determine a CNV profile as described herein. The profile is determined by any method described herein, e.g., by sequencing or by hybridization of genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In certain embodiments, the oligonucleotides for detecting CNV genetic markers associated with the duplication and deletion syndromes set forth herein may be used in high throughput sequencing methods (often referred to as next-generation sequencing methods or next-gen sequencing methods). Accordingly, in one embodiment, the present disclosure provides methods of determing or predicting the presence or absence of a deletion or duplication syndrome by detecting in a genetic sample from the subject one or more CNVs by high throughput sequencing. High throughput sequencing, or next-generation sequencing, methods are known in the art (see, e.g., Zhang et al., J Genet Genomics. 2011 Mar. 20; 38(3):95-109; Metzker, Nat Rev Genet. 2010 January; 11(1):31-46, incorporated by reference herein in its entirety) and include, but are not limited to, technologies such as ABI SOLiD sequencing technology (now owned by Life Technologies, Carlsbad, Calif.); Roche 454 FLX which uses sequencing by synthesis technology known as pyrosequencing (Roche, Basel Switzerland); IIlumina Genome Analyzer (Illumina, San Diego, Calif.); Dover Systems Polonator G.007 (Salem, N.H.); Helicos (Helicos BioSciences Corporation, Cambridge Mass., USA), and Sanger. In one embodiment, DNA sequencing may be performed using methods well known in the art including mass spectrometry technology and whole genome sequencing technologies (e.g., those used by Pacific Biosciences, Menlo Park, Calif., USA), etc.

In one embodiment, nucleic acid, for example, genomic DNA is sequenced using nanopore sequencing, to determine the presence of the one or more CNVs (e.g., as described in Soni et al. (2007). Clin Chem 53, pp. 1996-2001, incorporated by reference in its entirety for all purposes). Nanopore sequencing is a single-molecule sequencing technology whereby a single molecule of DNA is sequenced directly as it passes through a nanopore. A nanopore has a diameter on the order of 1 nanometer. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. patent application publications U.S. Patent Application Publication Nos. 2006/003171 and 2009/0029477, each incorporated by reference in its entirety for all purposes, is amenable for use with the methods described herein

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence. The probe sequence must also be sufficiently long so that the probe exhibits selectivity for the target sequence over non-target sequences. For example, the probe will be at least 10, e.g., 15, 20, 25, 30, 35, 50, 100, or more, nucleotides in length. In some embodiments, the probes are not more than 30, 50, 100, 200, 300, or 500 nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

Control probes can also be used. For example, a probe that binds a less variable sequence, e.g., repetitive DNA associated with a centromere of a chromosome, or a probe that exhibits differential binding to the polymorphic site being interrogated, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK).

In some embodiments, the probes are labeled with a detectable label, e.g., by direct labeling. In various embodiments, the oligonucleotides for detecting the one or more SNP genetic markers associated with ASD described herein are conjugated to a detectable label that may be detected directly or indirectly. In the present invention, oligonucleotides may all be covalently linked to a detectable label.

In one embodiment, CNV size is determined via a nucleic acid hybridization method as follows. Oligonucleotide probes are employed and each represents a known chromosomal coordinate based on hg19 coordinates. In a subject who has no deletion or duplication in a particular region, all probes specific to that region will have a uniform signal that represents having 2 copies of each chromosome at that position. A CNV is detected by looking for increases (duplication) or decreases (deletion) in signal intensity at individual probes, each of which represent a unique location in the genome. When 25 or more probes targeting contiguous regions of the genome show a reduced signal compared to an individual with no CNV, the test individual can then be said to have a deletion at the location containing the probes that have a reduced signal. Similarly, when 25 or more probes (for example 30 or more probes, or 50 or more probes) targeting contiguous regions of the genome show an increased signal compared to an individual with no CNV, the test individual can then be said to have a duplication at the location containing the probes that have an increased signal. Since the genomic coordinates of each probe are known, CNV size is determined by the coordinates of the probes showing reduced (in the case of a deletion) or increased (in the case of a duplication) signal intensity, and the maximal CNV boundaries are defined by the probes nearest to those showing reduced (deletion) signal or increased (duplication) signal that themselves do not show a reduced (deletion) signal or increased (duplication) signal.

For example, consider an example with oligonucleotide probes each having an arbitrary size of 1 unit for each probe. Probes 1-10 show a normal signal (e.g., as the probe is labeled with a detectable label), probes 11-67 show a reduced signal, and probes 68-1000 show a normal signal again. In this case, there is a deletion that is at least 56 units (67−11=56) in size, and at most 58 units in size (68−10). The CNV boundaries lie somewhere between probes 10 and 11 on the “left” end and between probes 67 and 68 on the “right” end. The same is true for a duplication, but one probes for an increase in signal intensity compared to a subject with no CNV, and duplications must include ≧0.50 probes to be detectable.

Where non-microarray based hybridization methods are employed to detect the presence or absence of a CNV, the size of the CNV can also be determined. For example, in a sequencing embodiment, the number of sequence reads of a particular sequence can be used to make a determination of whether a deletion or duplication occurs at the particular chromosomal location. Specifically, the number of sequence reads at a particular genomic DNA location can be compared to the number of sequence reads measured or that would be expected for a sample that does not include the CNV.

As provided above, an oligonucleotide probe or probes designed to hybridize a CNV or portion thereof can be labeled with a detectable label. A “detectable label” is a molecule or material that can produce a detectable (such as visually, electronically or otherwise) signal that indicates the presence and/or concentration of the label in a sample. When conjugated to a nucleic acid such as a DNA probe, the detectable label can be used to locate and/or quantify a target nucleic acid sequence to which the specific probe is directed. Thereby, the presence and/or amount of the target in a sample can be detected by detecting the signal produced by the detectable label. A detectable label can be detected directly or indirectly, and several different detectable labels conjugated to different probes can be used in combination to detect one or more targets.

Examples of detectable labels, which may be detected directly, include fluorescent dyes and radioactive substances and metal particles. In contrast, indirect detection requires the application of one or more additional probes or antibodies, i.e., secondary antibodies, after application of the primary probe or antibody. Thus, in certain embodiments, as would be understood by the skilled artisan, the detection is performed by the detection of the binding of the secondary probe or binding agent to the primary detectable probe. Examples of primary detectable binding agents or probes requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to the oligonucleotides used in the methods of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (α.-NP), o-dianisidine (OD), 5-bromo-4-chloro-3-indolylphosphate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to any molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents, nucleic acids, or antibodies, etc. Moreover, the skilled artisan would appreciate that each additional binding agent or nucleic acid used to characterize a biological marker of interest (e.g., the CNV genetic markers associated with ASD) may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

In another aspect, the invention features arrays that include a substrate having a plurality of addressable areas, and methods of using them. At least one area of the plurality includes a nucleic acid probe that binds specifically to a sequence comprising a CNV, for example one of the chromosomal locations set forth at Tables A and/or B, or one or more CNVs set forth in one or more of Tables 8-10 and 12-13, or a CNV associated with one or more of the genes set forth at Table 15, and can be used to detect the absence or presence of the CNV, and the size of the CNV, as described herein. The substrate can be, e.g., a two-dimensional substrate known in the art such as a glass slide, a wafer (e.g., silica or plastic), a mass spectroscopy plate, or a three-dimensional substrate such as a gel pad. In some embodiments, the probes are nucleic acid capture probes.

Methods for generating arrays are known in the art and include, e.g., photolithographic methods (see, e.g., U.S. Pat. Nos. 5,143,854; 5,510,270; and 5,527,681, each of which is incorporated by reference in its entirety), mechanical methods (e.g., directed-flow methods as described in U.S. Pat. No. 5,384,261), pin-based methods (e.g., as described in U.S. Pat. No. 5,288,514, incorporated by reference in its entirety), and bead-based techniques (e.g., as described in PCT US/93/04145, incorporated by reference in its entirety). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. After hybridization and washing, the array is scanned to determine the position on the array to which the nucleic acid from the sample hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms, e.g., distinct polymorphisms at the same polymorphic site or polymorphisms at different chromosomal sites. Detection blocks may be grouped within a single array or in multiple, separate arrays so that varying conditions (e.g., conditions optimized for particular polymorphisms) may be used during the hybridization.

Additional description of use of oligonucleotide arrays for detection of polymorphisms can be found, for example, in U.S. Pat. Nos. 5,858,659 and 5,837,832, each of which is incorporated by reference in its entirety.

Results of the CNV profiling performed on a sample from a subject (test sample) may be compared to a biological sample(s) or data derived from a biological sample(s) that is known or suspected to be normal (“reference sample” or “normal sample”). In some embodiments, a reference sample is a sample that is not obtained from an individual having deletion or duplication syndrome, or would test negative in the particular one or more CNVs probed for in the test sample. The reference sample may be assayed at the same time, or at a different time from the test sample.

The results of an assay on the test sample may be compared to the results of the same assay on a reference sample. In some cases, the results of the assay on the reference sample are from a database, or a reference. In some cases, the results of the assay on the reference sample are a known or generally accepted value or range of values by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, CNV presence or absence, CNV size.

In one embodiment, an odds ratio (OR) is calculated for each individual CNV measurement. Here, the OR is a measure of association between the presence or absence of an SNP, and an outcome, e.g., deletion or duplication syndrome positive or negative, or likely to respond to therapy for the respective deletion or duplication syndrome. Odds ratios are most commonly used in case-control studies. For example, see, J. Can. Acad. Child Adolesc. Psychiatry 2010; 19(3): 227-229, which is incorporated by reference in its entirety for all purposes. Odds ratios for each CNV can be combined to make an ultimate diagnosis, to select a patient for treatment of a deletion or duplication syndrome, or to predict whether a subject is likely to respond to therapy for a deletion or duplication syndrome, for example, a deletion or duplication syndrome associated with developmental delay.

In one embodiment, a specified statistical confidence level may be determined in order to provide a diagnostic confidence level. For example, it may be determined that a confidence level of greater than 90% may be a useful predictor of the presence of a deletion or duplication syndrome, or to predict whether a subject is likely to respond to therapy for a deletion or duplication syndrome. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and/or the number of CNVs analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operating Characteristic (ROC) curve analysis, binomial ROC, principal component analysis, odds ratio analysis, partial least squares analysis, singular value decomposition, least absolute shrinkage and selection operator analysis, least angle regression, and the threshold gradient directed regularization method.

CNV detection may in some cases be improved through the application of algorithms designed to normalize and or improve the reliability of the data. In some embodiments of the present disclosure the data analysis requires a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a CNV profile. The signals corresponding to certain CNVs, which are obtained by, e.g., microarray-based hybridization assays, sequencing assays, NanoString assays, etc., are in one embodiment subjected to the algorithm in order to classify the profile. Supervised learning generally involves “training” a classifier to recognize the distinctions among classes (e.g., CNV present, CNV absent, deletion syndrome positive, deletion syndrome negative, duplication syndrome positive, duplication syndrome negative) and then “testing” the accuracy of the classifier on an independent test set. For new, unknown samples the classifier can be used to predict the class (e.g., CNV present, CNV absent, deletion syndrome positive, deletion syndrome negative, duplication syndrome positive, duplication syndrome negative) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2): 249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety for all purposes) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety). Confidence intervals, in one embodiment, are computed using the pROC package (Robin X, Turck N, Hainard A, et al. pROC: an open-source package for R and S+ to analyze and compare ROC curves. BMC bioinformatics 2011; 12: 77, incorporated by reference in its entirety).

In addition, data may be filtered to remove data that may be considered suspect. In one embodiment, data derived from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may be considered unreliable due to their aberrant hybridization propensity or secondary structure issues.

In some embodiments of the present invention, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N−1) degrees of freedom. (N−1)*Probe-set Variance/(Gene Probe-set Variance). about.Chi-Sq(N−1) where N is the number of input CEL files, (N−1) is the degrees of freedom for the Chi-Squared distribution, and the “probe-set variance for the gene” is the average of probe-set variances across the gene. In some embodiments of the present invention, probe-sets for a given CNV or group of CNVs may be excluded from further analysis if they contain less than a minimum number of probes that pass through the previously described filter steps for GC content, reliability, variance and the like. For example in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of CNV data analysis in one embodiment, further include the use of a feature selection algorithm as provided herein. In some embodiments of the present invention, feature selection is provided by use of the LEMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of CNV data analysis, in one embodiment, include the use of a pre-classifier algorithm. For example, an algorithm may use a specific molecular fingerprint to pre-classify the samples according to their composition and then apply a correction/normalization factor. This data/information may then be fed in to a final classification algorithm which would incorporate that information to aid in the final diagnosis.

Methods of CNV data analysis, in one embodiment, further include the use of a classifier algorithm as provided herein. In one embodiment of the present invention, a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples (e.g., CNV duplication present vs. CNV duplication absent; CNV deletion present vs. CNV deletion absent; CNV size “n” vs. CNV size “x”, where “x” and “n” are the length in bases or basepairs of the CNV) are selected based on statistical significance of the difference in expression levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of microarray data are known in the art and have been described for example in Smyth, G. K. 2004 Stat. Appi. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the molecular profiling may provide a quantitative value or values indicative of one or more of the following: the likelihood of the presence or absence of one or more CNVs; the likelihood of diagnostic accuracy of a deletion or duplication syndrome; the likelihood of a particular deletion or duplication syndrome; the likelihood of the success of a particular therapeutic intervention. In one embodiment, the data is presented directly to the physician in its most useful form to guide patient care, or is used to define patient populations in clinical trials or a patient population for a given medication. The results of the molecular profiling can be statistically evaluated using a number of methods known to the art including, but not limited to: the students T test, the two sided T test, pearson rank sum analysis, hidden Markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some cases the results of the CNV detection and sizing assays, are entered into a database for access by representatives or agents of a molecular profiling business, the individual, a medical provider, or insurance provider. In some cases assay results include sample classification, identification, or diagnosis by a representative, agent or consultant of the business, such as a medical professional. In other cases, a computer or algorithmic analysis of the data is provided automatically. In some cases the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the CNV detection and sizing assays are presented as a report on a computer screen or as a paper record. In some embodiments, the report may include, but is not limited to, such information as one or more of the following: the number of CNVs identified as compared to the reference sample, the size of a CNV identified as compared to the size of the CNV in a reference sample (or reference database), the suitability of the original sample, a diagnosis, a statistical confidence for the diagnosis, the likelihood of a particular deletion or duplication syndrome, and proposed therapies.

The results of the CNV profiling may be classified into one of the following: CNV positive, CNV size (if CNV positive), CNV negative, deletion syndrome positive, deletion syndrome negative, non-diagnostic (providing inadequate information concerning the presence or absence of one or more CNVs or the size of one or more CNVs).

In some embodiments of the present invention, results are classified using a trained algorithm. Trained algorithms of the present invention include algorithms that have been developed using a reference set of known CNV and/or normal samples, for example, samples from individuals diagnosed with a particular deletion or duplication syndrome, or not diagnosed with the deletion or duplication syndrome. In some embodiments, training comprises comparison of one or more CNVs (presence and optionally size) in from a first CNV positive sample to the one or more CNVs in a second ASD positive sample, where the first set of CNVs include at least one CNV that is not in the second set.

Algorithms suitable for categorization of samples include but are not limited to k-nearest neighbor algorithms, support vector machines, linear discriminant analysis, diagonal linear discriminant analysis, updown, naive Bayesian algorithms, neural network algorithms, hidden Markov model algorithms, genetic algorithms, or any combination thereof.

When classifying a biological sample for diagnosis of a deletion or duplication syndrome, for example, WHS, or for the selection of a patient for treatment of a deletion or duplication syndrome, there are typically two possible outcomes from a binary classifier. When a binary classifier is compared with actual true values (e.g., values from a biological sample), there are typically four possible outcomes. If the outcome from a prediction is p (where “p” is a positive classifier output, such as the presence of a deletion or duplication syndrome) and the actual value is also p, then it is called a true positive (TP); however if the actual value is n then it is said to be a false positive (FP). Conversely, a true negative has occurred when both the prediction outcome and the actual value are n (where “n” is a negative classifier output, such as no deletion or duplication syndrome), and false negative is when the prediction outcome is n while the actual value is p. In one embodiment, consider a diagnostic test that seeks to determine whether a person has a certain deletion or duplication syndrome. A false positive in this case occurs when the person tests positive, but actually does not have the deletion or duplication syndrome. A false negative, on the other hand, occurs when the person tests negative, suggesting they are healthy, when they actually do have the disease (the deletion or duplication syndrome).

The positive predictive value (PPV), or precision rate, or post-test probability of disease, is the proportion of subjects with positive test results who are correctly diagnosed. It reflects the probability that a positive test reflects the underlying condition being tested for. Its value does however depend on the prevalence of the disease, which may vary. In one example the following characteristics are provided: FP (false positive); TN (true negative); TP (true positive); FN (false negative). False positive rate (□)=FP/(FP+TN)-specificity; False negative rate (□)=FN/(TP+FN)-sensitivity; Power=sensitivity=1−□□; Likelihood-ratio positive=sensitivity/(1−specificity); Likelihood-ratio negative=(1−sensitivity)/specificity. The negative predictive value (NPV) is the proportion of subjects with negative test results who are correctly diagnosed.

In some embodiments, the results of the CNV analysis of the subject methods provide a statistical confidence level that a given diagnosis is correct. In some embodiments, such statistical confidence level is at least about, or more than about 85%, 90%, 91%, 92%, 93%, 94%, 95%, 96%, 97%, 98%, 99% 99.5%, or more.

In one embodiment, depending on the results of the CNV hybridization assay and data analysis, the subject is selected for treatment for a particular deletion or duplication syndrome.

The present invention relates to diagnostic tests for determining whether a subject has a deletion or duplication syndrome, or predicting the presence or absence of one or more of the deletion or duplication syndromes set forth in Tables A and B. The diagnostic tests described herein may be an in vitro diagnostic test. Diagnostic tests include but are not limited to FDA approved, or cleared, In Vitro Diagnostic (IVD), Laboratory Developed Test (LDT), or Direct-to-Consumer (DTC) tests, that may be used to assay a sample and detect or indicate the presence of, the predisposition to, or the risk of, diseases, disorders, conditions, infections and/or therapeutic responses. In one embodiment, a diagnostic test may be used in a laboratory or other health professional setting. In another embodiment, a diagnostic test may be used by a consumer at home. Diagnostic tests comprise one or more reagents for detecting the presence or absence of the one or more CNV genetic markers associated with the particular deletion or duplication syndrome and may comprise other reagents, instruments, and systems intended for use in the in vitro diagnosis of disease or other conditions, including a determination of the state of health, in order to cure, mitigate, treat, or prevent disease. In one embodiment, the diagnostic tests described herein may be intended for use in the collection, preparation, and examination of specimens taken from the human body. In certain embodiments, diagnostic tests and products may comprise one or more laboratory tests. As used herein, the term “laboratory test” means one or more medical or laboratory procedures that involve testing samples of blood, urine, or other tissues or substances in the body.

One aspect of the present invention comprises an in vitro test for determining the presence or absence of a deletion or duplication syndrome, or predicting the likelihood of a deletion or duplication syndrome in a subject comprising a reagent for detecting one or more CNV genetic markers associated with the deletion or duplication syndrome, wherein the at least one CNV genetic marker comprises: at least one CNV genetic marker present at the chromosome location set forth in Table A or Table B, or at least one CNV as set forth in Tables 3-4, 8-10, 12 and/or 13; wherein detection in a genetic sample from the subject of the at least one CNV indicates that the individual is affected with the deletion or duplication syndrome, or is predisposed to developing the deletion or duplication syndrome.

In one embodiment the at least one CNV in Table A or Table B, or at least one CNV as set forth in Tables 3-4, 8-10, 12 and/or 13 comprises one or more of the CNV genetic markers numbered 6, 8, 10, 16 and 22 in Table 3.

In one embodiment, a diagnostic test as described herein has a diagnostic yield for the deletion or duplication syndrome of about 8% to about 40%. Diagnostic yield refers to the percent of individuals with the diagnosis of ASD that will have an abnormal genetic test result and is equal to sensitivity. In this regard, the diagnostic test described herein may have a diagnostic yield for ASD of about 8% to about 14%, from about 9% to about 13%, or from about 10% to about 12%. In further embodiments, a diagnostic test as described herein has a diagnostic yield for ASD of at least about 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or at least about 40%.

In certain embodiments, the CNV genetic markers associated with ASD as described herein may be isolated, amplified, and/or cloned into a vector. The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention (e.g., an isolated nucleic acid containing a CNV associated with ASD as described herein) can be inserted into a vector by cutting the vector with restriction enzymes and ligating the two pieces together.

Many techniques are available to those skilled in the art to facilitate transformation, transfection, or transduction of an expression construct into a prokaryotic or eukaryotic organism. The terms “transformation”, “transfection”, and “transduction” refer to methods of inserting a nucleic acid and/or expression construct into a cell or host organism. These methods involve a variety of techniques known to the skilled artisan, such as treating the cells with high concentrations of salt, an electric field, or detergent, to render the host cell outer membrane or wall permeable to nucleic acid molecules of interest, microinjection, PEG-fusion, and the like.

Those skilled in the art will recognize that a nucleic acid vector can contain nucleic acid elements other than the promoter element and the autism specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

In one embodiment, the methods and in vitro diagnostic tests and products described herein may be used for the diagnosis of a deletion or duplication syndrome, patients with non-specific symptoms possibly associated with the deletion or duplication syndrome, and/or patients presenting with related disorders. In another embodiment, the methods and in vitro diagnostic tests described herein may be used for screening for risk of progressing from at-risk, non-specific symptoms possibly associated with the deletion or duplication syndrome, and/or fully-diagnosed ASD. In certain embodiments, the methods and in vitro diagnostic tests described herein can be used to rule out screening of diseases and disorders that share symptoms with the deletion or duplication syndrome. In yet another embodiment, the methods and in vitro diagnostic tests described herein may indicate diagnostic information to be included in the current diagnostic evaluation in patients suspected of having the deletion or duplication syndrome.

In one embodiment, a diagnostic test may comprise one or more devices, tools, and equipment configured to collect a genetic sample from an individual. In one embodiment of a diagnostic test, tools to collect a genetic sample may include one or more of a swab, a scalpel, a syringe, a scraper, a container, and other devices and reagents designed to facilitate the collection, storage, and transport of a genetic sample. In one embodiment, a diagnostic test may include reagents or solutions for collecting, stabilizing, storing, and processing a genetic sample. Such reagents and solutions for collecting, stabilizing, storing, and processing genetic material are well known by those of skill in the art. In another embodiment, a diagnostic test as disclosed herein, may comprise a microarray apparatus and associated reagents, a flow cell apparatus and associated reagents, a multiplex next generation nucleic acid sequencer and associated reagents, and additional hardware and software necessary to assay a genetic sample for the presence of certain genetic markers and to detect and visualize certain genetic markers.

In certain embodiments, one or more CNV genetic markers described herein can be used in a method for selecting a patient for treatment of a mitochondrial associated disorder, or a disorder associated with a genetic duplication and/or deletion, for example, Wolf-Hirshhorn Syndrome (WHS). For example, the patient is selected for treatment of the deletion or duplication syndrome depending on the presence or absence of the particular CNV(s) that is probed for, and optionally, if the CNV(s) is present, the size of the CNV (e.g., as compared to a reference value) is taken into consideration in order to select the patient for therapy.

In one embodiment, the patient is selected for treatment with gene therapy, RNA interference (RNAi), behavioral therapy (e.g., Applied Behavior Analysis (ABA), Discrete Trial Training (DTT), Early Intensive Behavioral Intervention (EIBI), Pivotal Response Training (PRT), Verbal Behavior Intervention (VBI), and Developmental Individual Differences Relationship-Based Approach (DIR)), physical therapy, occupational therapy, sensory integration therapy, speech therapy, music therapy, the Picture Exchange Communication System (PECS), dietary treatment, or drug therapy (e.g., antipsychotics, anti-depressants, anticonvulsants, stimulants, aripiprazole, guanfacine, selective serotonin reuptake inhibitors (SSRIs), riseridone, olanzapine, naltrexone).

In the case of gene therapy treatment, in one embodiment, the gene therapy comprises delivery to the subject the wild type sequence of a particular gene that has been detected as part of a CNV in the patient.

Where a CNV that is associated with a mitochondrial gene is detected in a subject, the subject is selected for therapy with one or more of the following: EPI-743, antioxidants, oxygen, arginine, Coenzyme Q10, idebenone, benzoquinone therapeutics (e.g., alpha-tocotrien).

Where a CNV that is associated with glutamate or GABA receptor is detected in a subject, the subject, in one embodiment, is selected for therapy with a glutamate receptor agonist or antagonist or a GABA receptor agonist or antagonist. In a further embodiment, the subject is selected for therapy with a glutamatergic receptor agonist or GABAergic antagonist if the effect of the CNV is an inhibitory effect, and wherein the subject is administered a glutamatergic receptor antagonist or GABAergic agonist if the effect of the CNV is an excitatory effect.

### EXAMPLES

The present invention is further illustrated by reference to the following Example. However, it should be noted that these Examples, like the embodiments described above, are illustrative and are not to be construed as restricting the scope of the invention in any way. The references cited in the Example are incorporated by reference in their entireties for all purposes

### Example 1

### Identification of Rare Recurrent Copy Number Variants in High-Risk Autism Families and their Prevalence in a Large ASD Population

Genetics are known to play a major role in individuals with autism. However, the genetic underpinnings of autism are highly complex. The study described in this example used high-risk autism families to identify genetic variants that could predispose to autism in these families. This study also further evaluated these variants in a very large group of unrelated autism samples and controls to determine if these variants were relevant to children with autism in the broader population. This study identified 18 genetic variants that have not previously been observed in children with autism that are important not only in families but also in unrelated children with autism. By using a very large group of samples and controls this study also provides better frequency and significance estimates for many genetic variants previously associated with autism. This study sets the stage for using these genetic variants in the clinical analysis of children with autism.

Structural variation is thought to play a major etiological role in the development of ASDs, and numerous studies documenting the relevance of copy number variants in ASDs have been published since 2006. To determine if large ASD families harbor high-impact CNVs that may have broader impact in the general ASD population, the present experiments used the Affymetrix genome wide human SNP array 6.0 to identify 153 putative autism-specific CNVs present in 55 individuals with ASD from 9 multiplex ASD pedigrees. To evaluate the actual prevalence of these CNVs as well as 185 CNVs reportedly associated with ASD from published studies many of which are insufficiently powered, a custom Illumina array was designed and used to interrogate these CNVs in 3,000 ASD cases and 6,000 controls.

Additional single nucleotide variants (SNVs) on the array identified 25 CNVs not detected in the family studies at the standard SNP array resolution. After molecular validation, the results demonstrated that 15 CNVs identified in high-risk ASD families also were found in two or more ASD cases with odds ratios greater than 2.0, strengthening their support as ASD risk variants. In addition, of the 25 CNVs identified using SNV probes on the custom array, 9 also had odds ratios greater than 2.0, suggesting that these CNVs also are ASD risk variants. Eighteen of the validated CNVs have not been reported previously in individuals with ASD and three have only been observed once. Finally, the results described here confirmed the association of 31 of 185 published ASD-associated CNVs in this dataset with odds ratios greater than 2.0, suggesting they may be of clinical relevance in the evaluation of children with ASDs. Taken together, these data provide strong support for the existence and application of high-impact CNVs in the clinical genetic evaluation of children with ASD.

Twin studies [1-3], (reviewed in [4]), family studies [5-7], and reports of chromosomal aberrations in individuals with ASD (reviewed in [8]) all have strongly suggested a role for genes in the development of ASD. Although the magnitude of the genetic effect observed in ASD varies from study to study, it is clear that genetics plays a significant role.

While a number of genes associated with ASD susceptibility have been observed in multiple studies, variants in a single gene cannot explain more than a small percentage of cases. Indeed, recent estimates suggest that there may be nearly 400 genes or chromosomal regions involved in ASD predisposition [9-12].

In the past few years, a number of studies have identified both de novo and inherited structural variants, CNVs, that are associated with ASD [13-23]. De novo CNVs may explain at least some of the “missing heritability” of ASD as understood to date. While it is clear that CNVs play an important role in susceptibility to ASD, it is also clear that the genetic penetrance of many of these CNVs is less than 100%. Although many of the duplications or deletions observed in children with ASD occur as de novo variants, duplications, for example on chromosome 16p11.2, often are inherited from an asymptomatic parent. Moreover, both deletions and duplications encompassing a portion of chromosome 16p11.2 have been associated with ASD [21, 24-26] and 16p11.2 gains have been associated with ADHD and schizophrenia [24, 27-29], indicating that the same genomic region can be involved in multiple developmental conditions. In addition, deletions on chromosome 7q11.23 are known to cause Williams syndrome and duplications of this same region have been observed and are thought to be causal in individuals with ASD [9,11]. While individuals with Williams syndrome tend to be outgoing and social, individuals with ASD are socially withdrawn, suggesting that deletions and duplications in this region result in individuals on opposite sides of the behavioral spectrum.

Although numerous studies regarding the role of CNVs in ASD have been published in the research literature, the findings of these studies have not been fully utilized for clinical evaluation of children with ASD. This is likely due to the rarity of individual variants, the lack of probe coverage on clinical microarrays that permits detection of smaller variants, and the difficulty in understanding the relevant biology of some variants even when they are significantly associated with ASD. Despite this, published clinical guidelines suggest that microarray-based testing should be the first step in the genetic analysis of children with syndromic and non-syndromic ASD as well as other conditions of childhood development [30], and there is a wealth of information demonstrating its utility in large samples of children who have undergone such testing [25,31].

This example describes efforts to discover high-impact CNVs in high-risk ASD families in Utah and to assess their potential role in unrelated ASD cases. These CNVs were interrogated, as well as CNVs from multiple published sources [18,32] in a large sample set of ASD cases and controls, to determine more precisely their potential disease relevance. To evaluate carefully these CNVs, a custom Illumina iSelect array was designed containing probes within and flanking CNV regions of interest. This custom array was used to obtain high-quality CNV results on 2,175 children with clinically diagnosed ASD and 5,801 children with normal development following removal of samples that did not meet stringent quality control parameters. The results of this study identify multiple rare recurrent CNVs from high-risk ASD families that also confer risk in unrelated ASD cases and delineate the prevalence and impact of CNVs reported in the literature in a large case control study of ASDs.

DNA Samples.

DNA samples from high-risk ASD family members were collected after obtaining informed consent using a University of Utah IRB-approved protocol. Three independent sample cohorts, comprising 3,000 ASD patient samples (72% male), were collected for CNV replication. Of those, 857 were probands recruited and genotyped by the Center for Applied Genomics (CAG) at The Children's Hospital of Philadelphia (CHOP) from the greater Philadelphia area using a CHOP IRB-approved protocol; 2,143 ASD samples were from the AGRE and the AGP consortium (Rutgers, N.J. ASD repository), and genotyped at the CAG center at CHOP (Table 1). Only samples from affected individuals diagnosed using the Autism Diagnostic Interview-Revised (ADI-R) and the Autism Diagnostic Observation Schedule (ADOS) were used in the study. All control samples were from CHOP and were matched in a 2:1 ratio with the ASD cases.

TABLE 1
Case and control samples used in this study.

| | |
|---|---|
| case | control |

| | | female | male | female |
|---|---|---|---|---|
| AGRE/AGP | 1,517 | 626 | 0 | 0 |
| CHOP | 633 | 224 | 3,992 | 2,008 |
| sub-total | 2,150 | 850 | 3,992 | 2,008 |

| | | |
|---|---|---|
| grand-total | 3,000 | 6,000 |

CNV Discovery in High-Risk ASD Families.

DNA samples were genotyped on the Affymetrix Genome-Wide Human SNP Array 6.0 according to the manufacturer's protocol. Fifty-five autism subjects were chosen from 9 families with multiple affected first-degree relatives. The number of individuals with an autism diagnosis in these families ranged from 3 to 9. Affected individuals were diagnosed using ADI-R and ADOS. Control subjects (N=439) for the discovery phase of the project were selected from Utah CEPH/Genetics Reference Project (UGRP) families [70]. All microarray experiments were performed on blood DNA samples, except for two of the 55 case samples and three control subjects for which DNA from lymphoblastoid cell lines was used. CNVs were initially detected using the Copy Number Analysis Module (CNAM) of Golden Helix SNP & Variation Suite (SVS) (Golden Helix Inc.). Log ratios were calculated by quantile normalizing the A allele and B allele intensities using the entire population as a reference median for each SNP.

Batch effects in the log ratios were corrected via numeric principle component analysis (PCA) [71]. CNV segmentation analysis was carried out for each individual using the univariate CNAM segmentation procedure of Golden Helix SVS. We used a moving window of 5,000 markers, maximum number of segments per window of 20, minimum segment size 10 markers, and pairwise permutation p-value of 0.001.

iSelect Array Design.

Probes for each CNV to be characterized in this study were selected from the Illumina Omni2.5 array probe set. Probes were selected to be as uniformly spaced across each region and flanking each region as possible (using the hg19 genome build). For each CNV, we included 10 or more probes within the defined CNV region (CNVr) and five probes on each flank (except where not possible due to the telomeric location of a CNVr). Probes for an additional 185 CNVs described in the literature, including 104 identified by CHOP in samples that partially overlap those used in this study, also were included for further CNV validation. We attempted to increase probe coverage for CNVs identified with only a small number of probes. Probes for 2,799 putative functional candidate SNVs detected by targeted exome DNA sequencing on 26 representative individuals from 11 ASD families (unpublished data) were included. The genes that were targeted for exome sequencing included all known genes in regions of familial haplotype sharing and linkage as well as additional autism candidate genes. These SNVs, although included in a search for potential ASD point mutations, also were used to identify additional CNVs.

Array Processing.

High throughput SNP genotyping using the Illumina Infinium™ II BeadChip technology (Illumina, San Diego), at the Center for Applied Genomics at CHOP was performed. Detailed methods for array processing are described in the section entitled Supplemental Materials below.

CNV Calling and Statistical Analysis.

CNVs were called using both PennCNV [34,35] and CNAM (Golden Helix SNP & Variation Suite (SVS), Golden Helix, Inc.). CNV calling using PennCNV was performed as described [32]. For CNAM calls, each target region was separately analyzed, rather than whole chromosomes. Since our array targeted specific regions and did not have probe coverage over much of the genome, it was desirable to avoid calling segments that spanned large regions with no data, and prevent any CNV calls from being influenced by distant data points. To accomplish this, the markers in the data set were grouped into “pseudochromosomes”, one for each CNV covered by the array, that were then considered individually in the segmentation algorithm. After segmentation, segments were classified as losses, gains, or neutral. Fisher's exact test was used to test for association of copy number loss versus no loss, and copy number gain versus no gain. Similar tests were conducted for the X chromosome, stratified by gender. Odds ratios also were calculated as an indicator of potential clinical risk for each CNV.

Laboratory Confirmation of CNVs.

Array results were confirmed using pre-designed Applied Biosystems TaqMan copy number assays or custom-designed TaqMan copy number assays when necessary (Life Technologies, Inc.). All CNVs with odds ratios greater than 2.0 and present in at least two cases were selected for molecular validation. We did not select CNVs with odds ratios less than 2 were not selected for validation because these odds ratios were not thought to have high potential clinical utility. Six CNVs were also selected for validation because they were adjacent to, but not overlapping, literature CNVs that were covered by probes on the custom array. A maximum of 6 case samples were validated for each CNV. Five negative control samples, selected based on their lack of all of the CNVs under study also were included in each validation assay. A list of all of the TaqMan assays used in this work is found in Table 7, and detailed procedures of the TaqMan assays are described in the supplemental methods.

Pathway Analysis.

Analysis of biological pathways encompassing genes found in the CNV regions was performed using the bioinformatics tools DAVID Bioinformatics Resources 6.7 [72,73] and Ingenuity Pathways Analysis (IPA) (Ingenuity® Systems). Network and pathway analyses on genes contained within the CNVs or immediately flanking intergenic CNVs that were PCR validated was performed. Pathway analysis details are described in the supplemental methods.

CNV Discovery in Utah High Risk Autism Pedigrees.

Using CNAM (GoldenHelix Inc.) on Affymetrix Genome-Wide Human SNP array 6.0 data, a total of 153 CNVs in subjects with autism in Utah families that were not found in any CEPH/UGRP control samples were identified. This set included 131 novel CNVs and 22 CNVs present in the Autism Chromosomal Rearrangement Database [15]. Thirty-two autism-specific CNVs were detected in multiple (2 or more) autism subjects, and 121 CNVs were detected in only one person among the 55 autism subjects assayed. Of these, 153 CNVs, 112 were copy number losses (deletions) and 41 were copy number gains (duplications). The average size of the CNVs from high-risk families was 91 kb. The genomic locations of these CNVs are shown in Table 8.

CNV Regions on the Custom Array.

To better understand the frequency of the CNVs identified in Utah ASD families in a broader ASD population, we created a custom Illumina iSelect array containing probes covering all 153 of the Utah CNVs described in Table 8. CNV coordinate, copy number status, and probe content for each CNV are included. In addition, since the ultimate goal of this work is to understand the frequency and relevance of rare recurrent CNVs in the etiology of ASD, we included probes for 185 autism-associated CNVs identified in the literature [14-16, 18, 21, 32, 33] (Table 9). The probe coverage for each literature CNV also is shown in Table 9. In total, 7134 probes, all selected from the Illumina 2.5M array, were used for this study. As part of a separate study we also included 2799 SNVs detected by next-generation sequencing of genes in regions of haplotype sharing among our high-risk ASD families and in published ASD candidate genes in these same individuals also were included. Intensity data for these SNVs were used to identify additional CNVs that were not observed in our Utah high-risk ASD families (Table 10). Following standard data QC steps (see supplemental results) this array was used to characterize which of these 363 CNVs were present in DNA from 2,175 children with autism and 5,801 age, gender, and ethnicity matched controls (Table 1). These 7976 samples were available for analysis following our strict quality control measures (supplemental methods).

Analysis of CNVs on the iSelect Array.

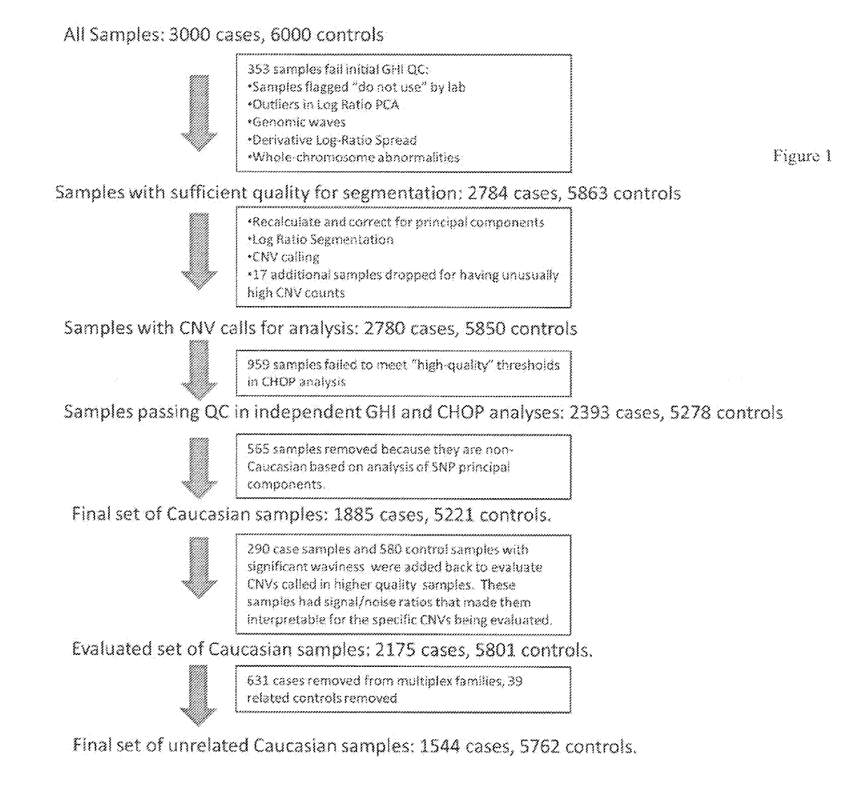

The workflow for CNV analyzis of the custom array data is shown in FIG. 1. Following quality control analysis, including removal of samples that did not meet laboratory sample quality control measures, samples with excessive CNV calls, samples of uncertain ethnicity, and related samples, our final dataset included 1544 unrelated cases and 5762 unrelated controls. Because of the inherent noisiness of CNV analysis, we used two independent CNV calling algorithms, PennCNV [34] and CNAM (Golden Helix, Inc.), to increase our ability to detect CNVs. We identified 6,086 CNVs in cases and 14,387 CNVs in controls using PennCNV and 3,226 CNVs in cases and 8,234 CNVs in controls using CNAM. 1,537 CNVs from the 2175 cases including those from multiplex families (average 0.70 CNVs per individual) and 3,845 CNVs from the 5801 controls including related controls (average of 0.66 CNVs per individual) were called by both algorithms used for CNV detection.

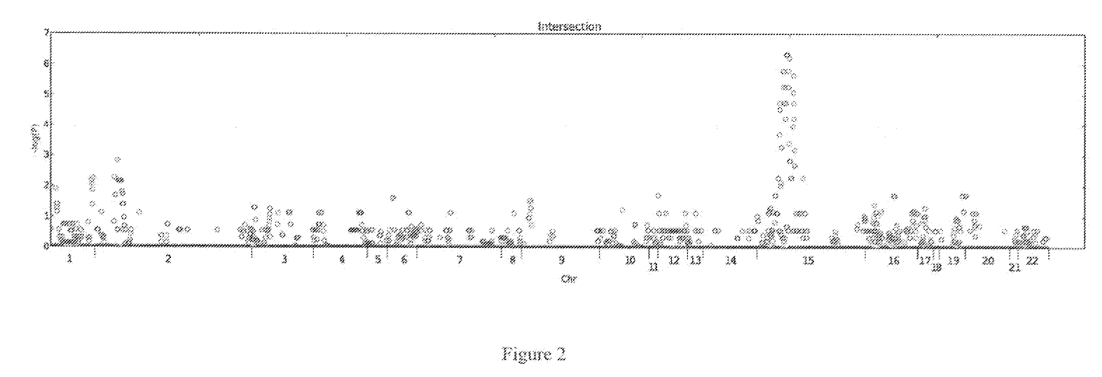

All CNV regions harboring CNVs shared among subjects were defined from PennCNV calls, CNAM calls and the PennCNV/CNAM intersecting calls and their significance of association was calculated across the genome (FIG. 2). Of the 153 CNVs discovered in high-risk ASD families, 139 of them were seen in replication samples evaluated with the custom Illumina iSelect array. Seven of the CNVs not seen in this larger population study had poor probe coverage on the array either due to their small size or their genomic content, while the remainder that were not detected may represent false positive CNVs from our initial discovery work or may be rare CNVs that are private to the families or individuals in which they were identified.

Molecular Validation of CNV Calls.

We used TaqMan copy number assays to confirm the presence of CNVs in our population. A summary of the 195 TaqMan assays used is shown in Table 7 (Hs assay names refer to assays available from Applied Biosystems, now Life Technologies, Carlsbad, Calif.). Since our goal for this study was to understand the frequencies of these CNVs in a large case/control population, we chose to validate any CNVs that were likely to have clinical relevance. Our criteria for selection were as follows: 1) any CNV with an odds ratio>=2.0; 2) any rare CNV seen in at least two cases. These criteria for selecting CNVs were chosen to validate because the goal was to translate research CNV findings into potentially clinically useful markers. Since clinical testing of individuals with ASD is only performed on people who are symptomatic, CNVs with odds ratios <1.0 (CNVs that indicate lower than average risk of ASD) were not chosen for validation. Likewise, since CNVs with odds ratios>=1 but <=2 do are not of great diagnostic interest, we chose to validate only CNVs with odds ratios>=2.0. By using these criteria, we included rare recurrent CNVs that may be etiologically important despite the lack of statistical significance in cases versus controls. For previously published CNVs we considered our custom Illumina iSelect array as an independent test of their validity. We assumed therefore that these CNVs did not require additional testing. Since some of the CNVs from CHOP were not included in previous publications [18,32], we selected all CHOP CNVs for molecular validation. For CNVs that met our selection criteria we assayed a maximum of six case samples that contained the CNV, giving priority to those samples called both by PennCNV and CNAM. Results of these TaqMan experiments are summarized in Table 2. Interestingly, many of the most common CNVs detected by the array were not validated by the TaqMan assays. For example, when we tested samples from a statistically significant CNV duplication on chromosome 7q36.1 that was detected only by PennCNV and not by CNAM, all samples tested were shown to have two copies rather than the anticipated three copies, suggesting that in this sample set at least some of the CNV duplications observed are not true positives. Conversely all but one of the CNVs observed on chromosome 15, whether in the Prader-Willi/Angelman syndrome region or located more distally on chromosome 15, were confirmed by TaqMan assays. Results of these validation experiments demonstrated that CNVs called both by PennCNV and CNAM were much more likely to be confirmed (97% of tested samples) than CNVs called by either PennCNV alone (24%) or CNAM alone (30%). This observation demonstrates the care that must be taken during the CNV discovery process to insure that only valid calls are selected for further analysis.

False negative results also are possible with these microarray studies. However, the controls used for TaqMan assays were selected from the control sample set because they lacked CNV calls for any of the regions being evaluated. In none of these samples did the TaqMan results indicate the presence of any of the CNVs being validated, so no false negative results were detected. These data suggest that false negative results are not a common problem in this study.

TABLE 2
confirmation of CNV calls by quantitative PCR.

| TaqMan CNV | Utah Family | Utah Sequence | Literature | |
|---|---|---|---|---|
| Validation Status | CNVs | SNP CNVs | CNVs | Total |
| PASS | 24 (2 overlap | 15 | 25 | 64 |
| | with Lit. CNV) | | | |
| FAIL | 9 | 9 | 5 | 23 |
| NoCall | 0 | 1 | 0 | 1 |

A summary of the PCR validation result is shown. Sequence SNP CNVs were discovered in this work using SNVs present on this array for sequence variant confirmation in the same cohort.

CNVs from High-Risk Utah Families.

One hundred thirty-nine of the 153 CNVs identified in high-risk ASD families were observed in case and/or control samples in this large dataset. Of these, 33 were present in two or more cases and had odds ratios greater than 2 and thus were selected for molecular confirmation. Following TaqMan validation, fifteen of thirty-three CNVs were confirmed (Table 3). This set included 3 CNVs with mixed results (Table 3). A CNV that was validated in some samples but not in others was considered to have passed validation if the validated samples resulted in an odds ratio greater than 2.0 with at least two confirmed cases, even if other samples did not pass molecular validation. The remaining 18 CNVs did not pass validation experiments.

One hundred thirty-nine of the 153 CNVs identified in high-risk ASD families were observed in case and/or control samples in this large dataset. Of these, 33 were present in two or more cases and had odds ratios greater than 2 and thus were selected for molecular confirmation. Following TaqMan validation, fifteen of the thirty-three CNVs were validated (Table 3). Of the 15 validated CNVs identified in high-risk families, 4 were shown to be inherited CNVs while three were de novo CNVs in the discovery families. The remainder were of undetermined origin, in most cases due to lack of information for one or both parents. A CNV that was validated in some samples but not in others, for example if a CNV was validated in all calls made by both PennCNV and CNAM but was not validated in all calls made only by one program, was considered to have passed validation if the validated samples yielded an odds ratio greater than 2.0 with at least two cases confirmed by validation.

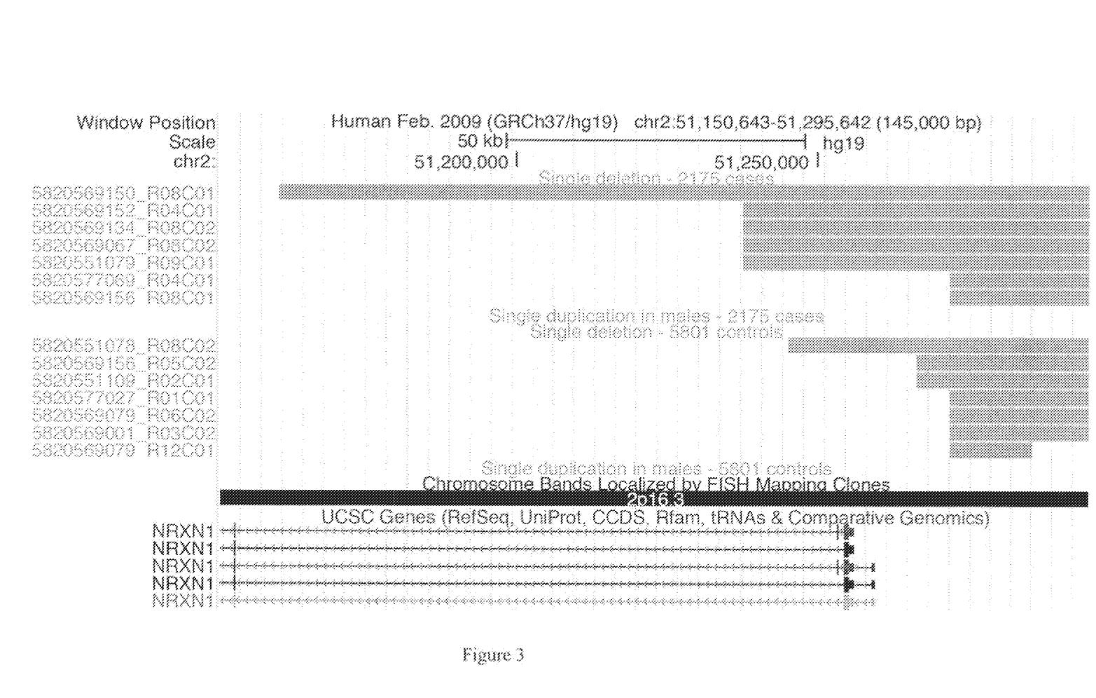

Notable among these CNVs is a deletion observed near the 5′-end of the NRXN1 gene. This deletion, observed in five cases and only in one control, includes at least a portion of the NRXN1-alpha promoter, and extends into the first exon of NLRXN1-α, as shown in the UCSC Genome Browser view [35] (FIG. 3). CNVs impacting NRXN1 in ASD as well as other neurological conditions have been published by others [15, 32, 36-40], so the observation of NRXN1 CNVs both in our high-risk ASD family discovery work and in the large case/control replication study demonstrates our ability to detect biologically relevant CNVs that may also have clinical utility.

Other CNVs of interest included portions of the LINGO2 and STXBP5 genes. Single nucleotide variants in the LINGO2 gene have been associated with essential tremor and with Parkinson's disease, suggesting that the LINGO2 protein may have a neurological function [41]. However, CNVs in this gene have not previously been identified in individuals with ASD. We also observed deletions involving a portion of the STXBP5 gene, an interesting finding based on the potential role of STXBP5 in neurotransmitter release [42,43].

CNVs Identified by SNV Probes.

Twenty-five additional CNVs shown in Table 3 were discovered using SNVs identified in our high-risk ASD families. The SNVs that detected these twenty-five CNVs (Table 10) were identified by exon capture and DNA sequencing in regions of haplotype sharing and in published ASD candidate genes in our high-risk ASD families, and were selected for further study because they might alter the function of the proteins in which they were found (unpublished observations). The 9 validated CNVs derived from SNV intensity data are shown in Table 3 (CNVs not detected in discovery cohort). One of these CNVs, a chromosome 15q duplication, encompasses three duplication CNVs in Table 10. These three CNVs are thought to be contiguous since TaqMan data confirmed the same samples to be positive for each of them.

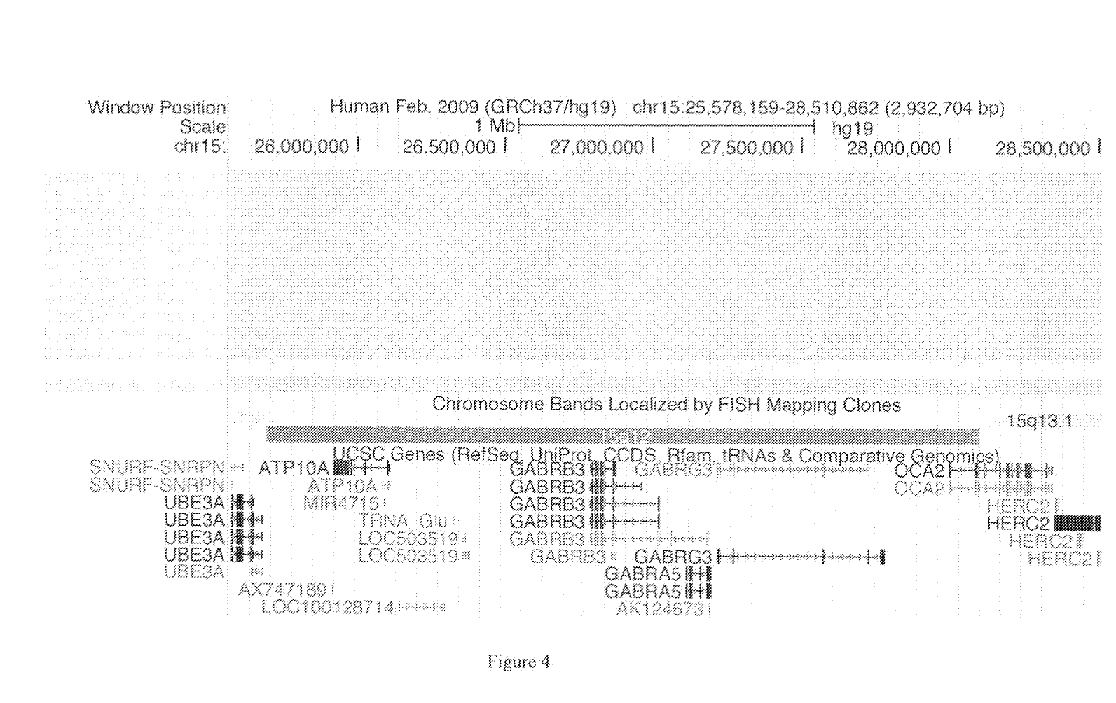

Interestingly, duplications involving the GABA receptor gene cluster, as well as many other genes, on chromosome 15q12 Were observed in 11 unrelated cases in our study and only in a single control, shown in the UCSC Genome Browser view [35] (FIG. 4). Contrary to our findings, a recent search for CNVs in GABA pathway genes [44] did not find an enrichment of duplications in this region. Rather, both deletions and duplications were observed at similar frequencies in cases and controls.

Published CNVs.

Additional CNVs from the literature and both published and unpublished CNVs identified at CHOP also were observed in our large dataset and met our criteria for potential clinical utility. Of those, 31 high-impact CNVs are shown in Table 4 (CNVs 20 and 21 in Table 4 are shown separately but are noted as likely being contiguous and thus likely are only a single entity). All CNVs not previously experimentally validated were validated in this study.

One of the previously unpublished CHOP CNVs is a duplication that encompasses the 3′-end RGS20 gene as well as the 3′-end of the TCEA1 gene. The RGS gene family encodes proteins that regulate G-protein signaling. These proteins function by increasing the inherent GTPase activity of their target G-proteins, and thus limit the signaling activity of their target G-proteins by keeping them in the inactive, GDP-bound state. RGS20 is expressed throughout the brain (reviewed in [45]), making it a likely candidate for involvement in neurological development. The TCEA1 gene, which also is partially encompassed by this CNV, is a transcription elongation factor involved in RNA polymerase II transcription. A role for TCEA1 in cell growth regulation has been suggested [46]. This potential role is consistent with the involvement of TCEA1 CNVs in ASD etiology as well.

TABLE 3
Validated CNVs discovered using affected children from Utah families

| | CNV | | CNV Region - | CNV Region - | CNV | Odds | | | | |
|---|---|---|---|---|---|---|---|---|---|---|
| No. | Origin | Cytoband | Discovery Cohort | Replication Cohort | Type | Ratio | P Value | Cases | Controls | Gene/Region |
| 1 | Utah CNV | 1q21.1 | chr1: 145714421- | chr1: 145703115- | Dup | 3.37 | 9.60E−03 | 9 | 10 | CD160, PDZK1 |
| | | | 146101228 | 145736438 | | | | | | |
| 2 | Utah CNV | 1q41 | chr1: 215858193- | chr1: 215854466- | Del | 2.12 | 5.02E−03 | 22 | 39 | USH2A |
| | | | 215861879 | 215861792 | | | | | | |
| 3 | Utah CNV | 2p16.3 | chr2: 51272055- | chr2: 51266798- | Del | 14.96 | 8.26E−03 | 4 | 1 | upstream of |
| | | | 51336043 | 51339236 | | | | | | NRXN1 |
| 4 | Utah CNV^{#} | 3q26.31 | chr3: 172596081- | chr3: 172591359- | Dup | 3.74 | 2.11E−01 | 1 | 1 | downstream of |
| | | | 172617355 | 172604675 | | | | | | SPATA16 |
| 5 | Utah CNV^{#} | 4q35.2 | chr4: 189084983- | chr4: 189084240- | Del | 3.74 | 1.98E−01 | 2 | 2 | downstream of |
| | | | 189117429 | 189117031 | | | | | | TRIML1 |
| 6 | Utah CNV^{#} | 6p24.3 | chr6: 7425246- | chr6: 7461346- | Del | ∞ | 2.11E−01 | 1 | 0 | between RIOK1 |
| | | | 7464367 | 7470321 | | | | | | and DSP |
| 7 | Utah CNV^{#} | 6q11.1 | chr6: 62443739- | chr6: 62426827- | Dup | 3.74 | 1.98E−01 | 2 | 2 | KHDRBS2 |
| | | | 62462295 | 62472074 | | | | | | |
| 8 | Utah CNV | 6q24.3 | chr6: 147588752- | chr6: 147577803- | Del | ∞ | 2.10E−01 | 1 | 0 | STXBP5 |
| | | | 147664671 | 147684318 | | | | | | |
| 9 | Utah CNV^{#} | 7p22.1 | chr7: 6838712- | chr7: 6870635- | Dup | 7.47 | 1.15E−01 | 2 | 1 | upstream of |
| | | | 6864071 | 6871412 | | | | | | CCZ1B |
| 10 | Sequence | 7q21.3 | Not found | chr7: 93070811- | Del | ∞ | 4.46E−02 | 2 | 0 | CALCR, MIR653, |
| | SNP CNV^{#} | | | 93116320 | | | | | | MIR489 |
| 11 | Utah CNV^{#} | 9p21.1 | chr9: 28190069- | chr9: 28207468- | Del | 3.74 | 6.72E−02 | 4 | 4 | LINGO2 |
| | | | 28347679 | 28348133 | | | | | | |
| 12 | Utah CNV^{#} | 9p21.1 | chr9: 28190069- | chr9: 28354180- | Del | 3.73 | 3.78E−01 | 1 | 1 | LINGO2 (intron) |
| | | | 28347679 | 28354967 | | | | | | |
| 13 | Utah CNV | 10q23.1 | chr10: 83893626- | chr10: 83886963- | Del | 3.76 | 1.54E−02 | 7 | 7 | NRG3 (intron) |
| | | | 84175018 | 83888343 | | | | | | |
| 14 | Utah CNV^{#} | 10q23.31 | chr10: 92274764- | chr10: 92262627- | Dup | 7.47 | 1.15E−01 | 2 | 1 | downstream of |
| | | | 92289762 | 92298079 | | | | | | BC037970 |
| 15 | Utah CNV^{#} | 12q23.2 | chr12: 102097012- | chr12: 102095178- | Dup | 7.47 | 1.15E−01 | 2 | 1 | CHPT1 |
| | | | 102106306 | 102108946 | | | | | | |
| 16 | Utah CNV# | 13q13.3 | chr13: 40087689- | chr13: 40089105- | Del | ∞ | 2.11E−01 | 1 | 0 | LHFP (intron) |
| | | | 40088007 | 40090197 | | | | | | |
| 17 | Sequence | 14q32.2 | Not found | chr14: 100705631- | Dup | 9.36 | 5.99E−03 | 5 | 2 | SLC25A29, YY1, |
| | SNP CNV^{#} | | | 100828134 | | | | | | MIR345, |
| | | | | | | | | | | SLC25A47, WARS |
| 18 | Sequence | 14q32.31 | Not found | chr14: 102018946- | Dup | 4.62 | 1.01E−14 | 60 | 50 | DIO3AS, DIO3OS |
| | SNP CNV^{#} | | | 102026138 | | | | | | |
| 19 | Sequence | 14q32.31 | Not found | chr14: 102729881- | Del | 7.47 | 1.15E−01 | 2 | 1 | MOK |
| | SNP CNV^{#} | | | 102749930 | | | | | | |
| 20 | Sequence | 14q32.31 | Not found | chr14: 102973910- | Dup | 3.82 | 8.29E−26 | 136 | 142 | ANKRD9 (RAGE) |
| | SNP CNV^{#} | | | 102975572 | | | | | | |
| 21 | Sequence | 15q11.2- | Not found | chr15: 25690465- | Dup* | 41.05 | 1.82E−08 | 11 | 1 | ATP10A, GABRB3, |
| | SNP CNV | q13.1 | | 28513763 | | | | | | GABRA5, GABRG3. |
| 22 | Sequence | 15q13.2- | Not found | chr15: 31092983- | Del | ∞ | 4.46E−02 | 2 | 0 | FAN1, MTMR10, |
| | SNP CNV^{#} | 15q13.3 | | 31369123 | | | | | | MIR211, TRPM1 |
| 23 | Sequence | 15q13.3 | Not found | chr15: 31776648- | Dup | 4.40 | 6.91E−06 | 21 | 18 | OTUD7A |
| | SNP CNV^{#} | | | 31822910 | | | | | | |
| 24 | Sequence | 20q11.22 | Not found | chr20: 32210931- | Dup | 2.72 | 3.16E−02 | 8 | 11 | NECAB3, CBFA2T2, |
| | SNP CNV^{#} | | | 32441302 | | | | | | C20orf144, NECAB3, |

CNVs shown here were selected based on their p value, their case/control odds ratio, or both and were subject to molecular validation.
*This CNV is contiguous with the chromosome 15q11.2 CNV described in Table 4 based on TaqMan data.
^{#}Designates CNVs not previously seen in ASD, based on queries for genes included in or flanking the CNV.
**Denotes gene in or adjacent to the CNV that is involved in neural function, development and disease (see Table 5-6).

TABLE 4
Published CNVs observed in the sample population

| | | | Region of | | | | | | | |
|---|---|---|---|---|---|---|---|---|---|---|
| | | Literature | Highest | CNV | TaqMan | | | | | |
| No. | Cytoband | CNVs | Significance | Type | Validation | OddsRatio | P Value | Cases | Ctrls | Gene/Region |
| 1 | 1q21.1 | chr1: 146555186- | chr1: 146656292- | Dup | NT | 7.48 | 1.15E−01 | 2 | 1 | FMO5 |
| | | 147779086 | 146707824 | | | | | | | |
| 2 | 2p24.3 | chr2: 13202218- | chr2: 13203874- | Del | Validated (chr2: | ∞ | 2.11E−01 | 1 | 0 | upstream of |
| | | 13248445 | 13209245 | | 13203874- | | | | | LOC100506474 |
| | | | | | 13209245) | | | | | |
| 3 | 2p21 | chr2: 45455651- | chr2: 45489954- | Dup | NT | ∞ | 4.46E−02 | 2 | 0 | between UNQ6975 |
| | | 45984915 | 45492582 | | | | | | | and SRBD1 |
| 4 | 2p16.3 | chr2: 50145644- | chr2: 51237767- | Del | NT | ∞ | 1.99E−03 | 4 | 0 | NRXN1** |
| | | 51259671 | 51245359 | | | | | | | |
| 5 | 2p15 | chr2: 62258231- | chr2: 62230970- | Dup | NT | ∞ | 2.11E−01 | 1 | 0 | COMMD1 |
| | | 63028717 | 62367720 | | | | | | | |
| 6 | 2q14.1 | chr2: 115139568- | chr2: 115133493- | Del | NT | 7.47 | 1.15E−01 | 2 | 1 | between |
| | | 115617934 | 115140263 | | | | | | | LOC440900 and |
| | | | | | | | | | | DPP10** |
| 7 | 3p26.3 | chr3: 1940192- | chr3: 1937796- | Del | Validated (chr3: | 5.60 | 6.70E−02 | 3 | 2 | between CNTN6 |
| | | 1940920 | 1941004 | | 1937796- | | | | | and CNTN4** |
| | | | | | 1942764) | | | | | |
| 8 | 3p14.1 | chr3: 67656832- | chr3: 67657429- | Del | NT | ∞ | 2.11E−01 | 1 | 0 | SUCLG2, FAM19A4, |
| | | 68957204 | 68962928 | | | | | | | FAM19A1 |
| 9 | 4q13.3 | chr4: 73756500- | chr4: 73766964- | Dup | Validated (chr4: | ∞ | 2.11E−01 | 1 | 0 | COX18, ANKRD17 |
| | | 73905356 | 73816870 | | 73753294- | | | | | |
| | | | | | 74058988) | | | | | |
| 10 | 4q33 | chr4: 154087652- | chr4: 171366005- | Del | NT | ∞ | 4.46E−02 | 2 | 0 | between AADAT** |
| | | 172339893 | 171471530 | | | | | | | and HSP90AA6P |
| 11 | 5q23.1 | chr5: 118478541- | chr5: 118527524- | Dup | Validated (chr5: | 3.74 | 1.98E−01 | 2 | 2 | DMXL1, TNFAIP8 |
| | | 118584821 | 118589485 | | 118527524- | | | | | |
| | | | | | 118614781) | | | | | |
| 12 | 6p21.2 | chr6: 39071841- | chr6: 39069291- | Del | Validated (chr6: | 2.37 | 1.93E−02 | 12 | 19 | SAYSD1 |
| | | 39082863 | 39072241 | | 39069291- | | | | | |
| | | | | | 39072241) | | | | | |
| 13 | 8q11.23 | chr8: 54858496- | chr8: 54855680- | Dup | Validated (chr8: | ∞ | 2.11E−01 | 1 | 0 | RGS20, TCEA1 |
| | | 54907579 | 54912001 | | 54855680- | | | | | |
| | | | | | 54912001) | | | | | |
| 14 | 10q11.22 | chr10: 46269076- | chr10: 49370090- | Dup | NT | 3.77 | 1.96E−01 | 2 | 2 | FRMPD2P1, |
| | | 50892143 | 49471091 | | | | | | | FRMPD2 |
| 15 | 10q11.23 | chr10: 50892146- | chr10: 50884949- | Dup | NT | 3.74 | 1.98E−01 | 2 | 2 | OGDHL, C10orf53 |
| | | 51450787 | 50943185 | | | | | | | |
| 16 | 12q13.13 | chr12: 53183470- | chr12: 53177144- | Del | Validated (chr22: | ∞ | 4.46E−02 | 2 | 0 | between KRT76 and |
| | | 53189890 | 53180552 | | 53177144- | | | | | KRT3 |
| | | | | | 53182177) | | | | | |
| 17 | 15q11.1 | chr15: 20266959- | chr15: 20192970- | Dup | Validated (chr15: | 4.97 | 4.06E−02 | 4 | 3 | downstream of |
| | | 25480660 | 20197164 | | 20192970- | | | | | HERC2P3 |
| | | | | | 20212798) | | | | | |
| 18 | 15q11.2 | chr15: 20266959- | chr15: 25099351- | Del | NT | 3.75 | 1.13E−01 | 3 | 3 | SNRPN** |
| | | 25480660 | 25102073 | | | | | | | |
| 19 | 15q11.2 | chr15: 20266959- | chr15: 25099351- | Dup | NT | 45.19 | 7.93E−08 | 12 | 1 | SNRPN** |
| | | 25480660 | 25102073 | | | | | | | |
| 20 | 15q11.2 | chr15: 25582397- | chr15: 25579767- | Dup* | Validated (chr15: | ∞ | 3.86E−06 | 8 | 0 | between |
| | | 25684125 | 25581658 | | 25576642- | | | | | SNORD109A and |
| | | | | | 25581880) | | | | | UBE3A** |
| 21 | 15q11.2 | chr15: 25582397- | chr15: 25582882- | Dup* | NT | 30.08 | 2.82E−05 | 8 | 1 | UBE3A** |
| | | 25684125 | 25662988 | | | | | | | |
| 22 | 16p12.2 | chr16: 21901310- | chr16: 21958486- | Dup | NT | ∞ | 4.47E−02 | 2 | 0 | C16orf52, |
| | | 22703860 | 22172866 | | | | | | | UQCRC2**, PDZD9, |
| | | | | | | | | | | VWA3A |
| 23 | 16p11.2 | chr16: 29671216- | chr16: 29664753- | Del | NT | 7.47 | 1.15E−01 | 2 | 1 | DOC2A**, ASPHD1, |
| | | 30173786 | 30177298 | | | | | | | LOC440356, TBX6, |
| | | | | | | | | | | LOC100271831, |
| | | | | | | | | | | PRRT2 |
| | | | | | | | | | | CDIPT, QPRT, YPEL3, |
| | | | | | | | | | | PPP4C, MAPK3**, |
| | | | | | | | | | | SPN, MVP, FAM57B, |
| | | | | | | | | | | ZG16, ALDOA, |
| | | | | | | | | | | INO80E, SEZ6L2, |
| | | | | | | | | | | TAOK2, KCTD13, |
| | | | | | | | | | | MAZ, KIF22, GDPD3, |
| | | | | | | | | | | C16orf92, C16orf53, |
| | | | | | | | | | | TMEM219, |
| | | | | | | | | | | C16orf54, HIRIP3 |
| 24 | 16q23.3 | chr16: 82195236- | chr16: 82423855- | Dup | NT | ∞ | 4.46E−02 | 2 | 0 | between |
| | | 82722082 | 82445055 | | | | | | | MPHOSPH6 and |
| | | | | | | | | | | CDH13 |
| 25 | 17p12 | chr17: 14139846- | chr17: 14132271- | Dup | Validated (chr17: | 1.60 | 3.57E−01 | 3 | 7 | between COX10 and |
| | | 15282723 | 14133349 | | 14132271- | | | | | CDRT15 |
| | | | | | 14133568) | | | | | |
| 26 | 17p12 | chr17: 14139846- | chr17: 14132271- | Del | NT | 5.61 | 6.70E−02 | 3 | 2 | PMP22**, CDRT15, |
| | | 15282723 | 15282708 | | | | | | | TEKT3, MGC12916, |
| | | | | | | | | | | CDRT7, HS3ST3B1 |
| 27 | 17p12 | chr17: 14139846- | chr17: 14952999- | Dup | NT | 3.74 | 1.98E−01 | 2 | 2 | between CDRT7 and |
| | | 15282723 | 15053648 | | | | | | | PMP22 |
| 28 | 17p12 | chr17: 14139846- | chr17: 15283960- | Del | Validated (chr17: | 3.74 | 1.13E−01 | 3 | 3 | between TEKT3 and |
| | | 15282723 | 15287134 | | 15283960- | | | | | FAM18B2-CDRT4 |
| | | | | | 15287134) | | | | | |
| 29 | 20p12.3 | chr20: 8044044- | chr20: 8162278- | Dup | NT | 3.73 | 1.98E−01 | 2 | 2 | PLCB1** |
| | | 8527513 | 8313229 | | | | | | | |
| 30 | Xp21.2 | chrX: 28605682- | chrX: 29944502- | Dup | NT | ∞ | 4.47E−02 | 2 | 0 | IL1RAPL1** |
| | | 29974014 | 29987870 | | | | | | | |
| 31 | Xq27.2 | chrX: 139998330- | chrX: 140329633- | Del | Validated (chrX: | 7.48 | 2.06E−02 | 4 | 2 | SPANXC |
| | | 140443613 | 140348506 | | 140329633- | | | | | |
| | | | | | 140456325) | | | | | |
| 32 | Xq28 | chrX: 148858522- | chrX: 148882559- | Del | Validated (chrX: | ∞ | 4.46E−02 | 2 | 0 | MAGEA8 |
| | | 149097275 | 148886166 | | 148882559- | | | | | |
| | | | | | 149020410) | | | | | |

*Denotes CNVs contiguous with the chromosome 15q11.2-13.1 CNVs shown in Table 3.
**Denotes gene in or adjacent to the CNV that is involved in neural function, development and disease (see Table 5-6).

Pathway Analysis.

Analysis of 104 genes within or immediately flanking our PCR-validated CNVs yielded significant association of these genes to previously characterized functional networks. The five most statistically significant networks, along with their statistical scores, are shown in Table 5. The top ranking functional categories identified in this analysis, along with their P-values, are shown in Table 6.

TABLE 5
Top Significant Networks Identified by
Pathway Analysis using Ingenuity IPA.

| Network | Score |
|---|---|
| Cell-To-Cell Signaling and Interaction, Tissue | 55 |
| Development, Gene Expression | |
| Neurological Disease, Behavior, Cardiovascular Disease | 28 |
| Cell Death, Cellular Compromise, Neurological Disease | 26 |
| Cellular Development, Cell Morphology, Nervous System | 20 |
| Development and Function | |
| Behavior, Cardiovascular Disease, Neurological Disease | 18 |

Network scores are the −log P for the results of a right-tailed Fisher's Exact Test.

As expected for CNVs associated with a neurodevelopmental disorder, a significant number of genes in or adjacent to the CNVs described here are involved in neural function, development and disease (Tables 5-6). Examples of such genes include: GABRA5, GABRA3, GABRG3, UBE3A, E2F1, PLCB1, PMP22, AADAT, MAPK3, NRXN1, NRG3, DPP10, UQCRC2, USH2A, NECAB3, CNTN4, LINGO2, IL1RAPL1, STXBP5, DOC2A, and SNRPN. Of these genes, E2F1, AADAT, NECAB3, and IL1RAPL1 are not found in the Autism Chromosome Rearrangement Database (see website at projects.tcag.ca/autism/), suggesting that they may be novel ASD risk genes.

The novel ASD risk loci identified here have functions that suggest a significant role in brain function and architecture. As such, altering the function of each of these genes as a result of the CNV could impinge on the biochemical pathways that are relevant to ASD etiology.

For example, mutations in IL1RAPL1 have been observed in cases of X-linked intellectual disability [47], and the encoded protein has been shown to play a role in voltage-gated calcium channel regulation in cultured cells [48]. E2F1 encodes a transcription factor and DNA-binding protein that plays a significant role in regulating cell growth and differentiation, apoptosis and response to DNA damage (reviewed in Biswas and Johnson, 2012 [49]). Each of these genes thus could have detrimental impacts on normal brain function.

NECAB3 encodes a neuronal protein with two isoforms that regulate the production of beta-amyloid peptide in opposite directions, depending on whether exon 9 of NECB3 is included in or excluded from the mature mRNA [50].

AADAT encodes an aminotransferase with multiple functions, one of which leads to the synthesis of kynurenic acid. This pathway has been proposed as a target for potential neuroprotective therapeutics, indicating the potential significance of this finding for ASD etiology (reviewed in Stone et al., 2012 [51]). The specific roles that any of these genes play in ASD etiology have yet to be determined, but the observed neurological functions of their encoded proteins strongly support a potential role in normal brain function.

Many of these genes also have been implicated in other nervous system disorders, including Huntington's, Parkinson's, and Alzheimer's diseases as well as schizophrenia and epilepsy [41, 52-61]. One of the features common to this group of disorders, which includes ASD, is synaptic dysfunction. There is a significant overlap in genes, and/or the molecular mechanisms by which these genes give rise to synaptopathies (reviewed in [62]). We therefore find it notable that many such genes involved in other synaptopathies were found within or flanking the validated CNVs we identified as associated with ASD.

In addition to neurogenic genes, validated CNVs were associated with genes with known roles in renal and cardiovascular diseases (Table 6). Several syndromic forms of autism, such as DiGeorge Syndrome and Charcot-Marie Tooth Disease are comorbid with renal and cardiovascular disease, and therefore it was not surprising to find that our study identified CNVs containing genes associated with these syndromes and functions, such as CDRT15, and CDH13.

TABLE 6
Top Significant Biological Functions Identified
by Ingenuity IPA and Literature Searches.

| Function | p-value range | # Genes |
|---|---|---|
| Neurological Disease | 2.71E−05-3.15E−02 | 14 (18) |
| Behavior | 5.93E−05-4.36E−02 | 10 |
| Cardiovascular Disease | 8.58E−05-4.30E−02 | 10 |
| Cellular Development | 1.39E−04-4.77E−02 | 9 |
| Inflammatory response | 4.84E−04-2.89E−02 | 6 |

The right-tailed Fisher's exact test was used to calculate P-values representing the probability that selecting genes associated with that pathway or network is due to chance alone. Each functional category represents a collection of associated subcategories, each of which has an associated P-value. For example, within ‘Neurological Disease,’ are subcategories of genes associated with seizures, Huntington Disease, schizophrenia, etc. The P-value range range given represents the range of P-values generated for each subcategory. In the first line, 36 genes were associated with a function in Neurological Disease by Ingenuity software. An additional 11 genes were identified as having neurological functions in the literature, giving a total of 47 with known or suspected roles in neurological disease.

There is mounting evidence, as well, that inflammatory responses are involved with the development and progression of autism (reviewed in [63]). Maternal immune activation during pregnancy is believed to activate fetal inflammatory responses, in some cases with detrimental effects on neural development in the fetus, leading to autism. This environmental insult could be mediated or enhanced by genomic changes that predispose the fetus to elevated inflammatory responses, so it is significant that a number of genes from our validated CNVs play a role in inflammatory response. Examples of these include CD160, CALCR, and SPN.

These findings are consistent with other studies that used pathway analysis to characterize the genes contained in ASD risk CNVs, and suggest that many different biological pathways, when disrupted, can lead to features observed in ASD. The wide variety of biological functions identified for these genes also is consistent with estimates of the number of independent genetic variants that may play a role in the etiology of ASD (8-11).

A custom microarray was used to characterize the frequency of CNVs identified in high-risk ASD families in a large ASD case/control population. We also evaluated further the frequency of CNVs discovered in several published studies in our sample cohort to obtain a clearer picture of the potential clinical utility of these CNVs in the genetic evaluation of children with ASD. Multiple quality control measures were used to insure that all cases and controls a) had no unexpected familial relationships; b) represented a uniform ethnic group; c) were devoid of uncharacterized whole chromosome anomalies or other genomic abnormalities consistent with syndromic forms of ASD; d) had sufficient power to distinguish risk variants from CNVs with little or no impact on the ASD phenotype; and e) were validated using quantitative PCR even though the custom array used here represented at least a second evaluation for most of them. Parents of ASD cases tested were not available to determine state of inheritance.

The validity of this approach was confirmed by our observation of CNVs that had been previously identified as ASD risked markers, including CNVs encompassing parts of the NRXN1 gene. CNVs and point mutations in NRXN1 are thought to play a role in a subset of ASD cases as well as in other neuropsychiatric conditions [15, 32, 36-40]. The data from our study demonstrate that NRXN1 CNVs also occur in high-risk ASD families. Further, our case/control data provide additional evidence that neurexin-1 plays an important role in unrelated ASD cases. While CNVs near NRXN1 occur in controls as well as in cases, the CVNs observed in our ASD cases typically disrupt a portion of the NRXN1 coding region while CNVs observed in our control population do not.

CNVs from High-Risk ASD Families.

In the high-risk ASD families, both novel and previously observed CNVs were identified that contain genes with potential relevance to neuropsychiatric conditions such as ASD. These include CNVs involving LINGO2, the GABR gene cluster on chromosome 15q12 and STXBP5. Each of these CNV regions has an odds ratio greater than 2 and most of the CNVs we identified in high-risk families have a significant p value associating them with the ASD phenotype in this case/control study. Some CNVs, although observed only in ASD cases and not in controls, were too rare even in this large dataset to generate statistically significant results. An example is a deletion involving STXBP5 that was observed two ASD samples and in no controls. A deletion including this gene was previously observed in a patient with an apparent syndromic form of ASD [64], lending further support to our observation of STXBP5 deletions in ASD cases. These data collectively suggest that CNVs observed in high-risk ASD families also are important contributors to the etiology of ASD in an ASD case/control population.

Rare duplications involving the GABA receptor gene cluster as well as additional genes in the Prader-Willi/Angelman syndrome region on chromosome 15 were detected (11/1,544 unrelated cases, 1/5,762 unrelated controls, OR=40.05). All of these CNVs were confirmed using TaqMan assays spanning the region, and these results strongly suggest a role for duplications on chromosome 15q12 in ASD etiology. Deficiency of GABA_{A} receptors indeed is thought to play an important role in both autism and epilepsy, and duplications have been observed to result in decreased GABR expression through a potential epigenetic mechanism (reviewed in [65]). Further, differences in the expression of GABRB3 mRNA and protein in the brains of some children with autism have been reported along with loss of biallelic expression of the chromosome 15q GABR genes in some individuals, [66], suggesting that epigenetic regulation of the chromosome 15 GABR gene cluster could also contribute to ASD etiology. Consistent with many previous findings from family studies, case reports and modest case/control studies (see website at omim.org/entry/608636), our data provide additional support for the involvement of duplications in this region of the genome in ASD. Further, the large population study suggests that these duplications may explain as much as 0.7% of ASD cases.

A recent study searching for CNVs encompassing genes in the GABA pathway, including the chromosome 15 GABR gene cluster, also found CNVs in this region. In contrast to our findings, this study found GABR gene cluster duplications at similar frequencies in both cases and in controls (Table S2 in ref. [44]). In addition, deletions were more common in this study in both cases and controls, while duplications were more common in our data. The differences between the two studies may lie in the sample population being studied, the uniformity of our sample population, or the technology platform used for CNV discovery (custom Illumina array compared to a custom Agilent array). Previous results have demonstrated maternal inheritance of deletions in this region in children with autism [67]. However, in our family studies we did not observe CNVs involving chromosome 15q12, and our case/control data preclude us from determining the parent of origin.

Interestingly, the CNVs that we observed on chromosome 15q were detected primarily with probes for SNVs identified in the GABR genes. Further, these SNVs were identified in affected individuals from high-risk ASD families. We did not observe CNVs involving this region in our high-risk ASD families. The observation of frequent duplications in our case/control population in the region containing these genes, coupled with the detection of these CNVs using probes for potential detrimental single nucleotide variants, suggests that both SNVs and CNVs involving the GABR genes might be pathogenic.

Literature Supported CNVs.

In addition to the CNVs identified in our high-risk ASD families, we evaluated further ASD risk CNVs identified in previous studies. Our results (Table 4) clearly demonstrate a role for many of these CNVs in ASD pathogenesis. Consistent with previous results, our data demonstrate in a large ASD population that rare CNVs are likely to play a role in the genetics of ASD, and suggest that these CNVs should be included in the Genetic evaluation of children with ASD.

Interestingly, recent publications have identified a recurrent duplication of the Williams syndrome region on chromosome 7q11.23 in children with ASD [9,11]. We included probes for this region on our custom array, and were not able to identify any 7q11.23 duplications in our datasets. The reason(s) we did not observe any duplications in this region is not obvious; we had adequate probe coverage to have seen such duplications if they were present. Similar to the simplex ASD families used in those published studies, most of our ASD samples also were from reported simplex families, so the lack of observation of these CNVs is unlikely to be due to differences in family structure.

A CNV discovered at CHOP and not previously published includes a portion of the LCE gene cluster on chromosome 1. Deletions in this region have been associated with psoriasis [68,69], but no variants in this region have been linked to autism. Focusing solely on individuals of Caucasian ancestry, we observed this CNV deletion in a single case and also a single control. However, when we included samples of non-Caucasian or uncertain ancestry, we observed 27 additional case DNA samples that carried this deletion, while only a single additional CNV-positive control was observed. Based on SNP genotype results from principal component analysis, all of the cases that were positive for this CNV were of Asian descent. Since our control cohort had few individuals of Asian descent, we suspected that this CNV might be common in the Asian population. Analysis of whole genome data for individuals of non-Caucasian ancestry genotyped at the Center for Applied Genomics did not demonstrate common CNVs in either cases or controls in this region in individuals with Asian ancestry. However, a common CNV including LCE3E was observed in individuals with African ancestry (unpublished observations). Further analysis will be necessary to determine if this CNV is an ASD risk variant in either Asian or African populations.

Effect of Analysis Method on CNV Validation.

Although some CNVs are described here for the first time, many of the CNVs that we evaluated in this study were described previously. It is interesting to note that individual CNV calls that were made with both of the software packages we used were much more likely to be validated by qPCR than were CNVs called by either program alone. In fact, 97% of the CNVs called by both PennCNV and CNAM validated using TaqMan qPCR assays, while only 24% of the CNVs called by PennCNV alone and 30% of the CNVs called by CNAM alone were validated using the same approach. The concordance between the two analysis methods is informative given that the final sample sets used by the two methods differed substantially. The CNAM analysis used 290 fewer case samples and 575 fewer control samples than the PennCNV analysis. These data clearly demonstrate the value of using multiple software packages to evaluate microarray data for CNV discovery work. Our data are consistent with the rarity of many CNVs detected in DNA from children with ASD, and with the suggestion that there may be hundreds of loci that contribute to the development of ASD [9,11].

These data demonstrate that CNVs identified in high-risk ASD families play a role in the etiology of ASD in unrelated cases. Evaluation of these CNVs in the large sample set used in this study provides compelling evidence for extremely rare recurrent CNVs as well as additional common variants in the genetics of ASD. We suggest that the CNVs described here likely have a strong impact on the development of ASD. Given the extensive quality control measures used to characterize the sample cohort, the frequency at which we observed these CNVs in our cohort, and the molecular validation that we used to verify the calls, these CNVs can be used to increase sensitivity in the genetic evaluation of children with ASD. Further work will help to determine if the CNVs reported here are important for specific clinical subsets of ASD cases.

Samples:

All high risk ASD family members and controls were of self-reported European ancestry. Among all cases in the replication study, 84% were of self-reported European ancestry, 6% were of self-reported African ancestry, 5% were self-reported as having multiple ethnic origins, and 5% were of unknown ethnicity. Among the cases, 1,577 were reported from unique families, 864 from 432 different families with 2 siblings, 369 from 123 different families with 3 siblings, 172 from 43 different families of 4 siblings, 5 siblings from a single family, 6 siblings from a single family, and 7 siblings from a single family. Among the DNA from cases used for genotyping, 1% came from cell pellets, 61% come from lymphoblastoid cell lines, 35% came from whole blood, and for 3% the source of DNA remained unknown. DNA was extracted from cell lines or lymphocytes, and quantitated using UV spectrophotometry. Six thousand controls were recruited by CHOP after obtaining informed consent under an IRB approved protocol. All DNA samples from controls were extracted from whole blood. Only individuals with self-reported Caucasian ancestry were used for this study. Pairwise identity by descent (IBD) was used to confirm known family assignments for cases, and to identify cryptic relatedness arising out of multiple subject enrollments across/within cohorts for all samples. Related individuals were removed so that only one family member remained in the study.

Array Processing:

We used 250 ng of genomic DNA to genotype each sample, according to the manufacturer's guidelines. On day one, genomic DNA was amplified 1000-1500-fold. Day two, amplified DNA was fragmented ˜300-600 bp, then precipitated and resuspended, followed by hybridization on to a BeadChip. Single base extension (SBE) utilizes a single probe sequence ˜50 bp long designed to hybridize immediately adjacent to the SNP query site. Following targeted hybridization to the bead array, the arrayed SNP locus-specific primers (attached to beads) were extended with a single hapten-labeled dideoxynucleotide in the SBE reaction. The haptens were subsequently detected by a multi-layer immunohistochemical sandwich assay, as recently described (Pastinen et al., 2000, Genome Res. 10, 1031, Erdogan et al., 2001, Nuc. Acids Res. 29, E36). The Illumina iScan was used to scan each BeadChip at two wavelengths and an image file was created. As BeadChip images were collected, intensity values were determined for all instances of each bead type, and data files were created that summarized intensity values for each bead type. These files were loaded directly into Illumina's genotype analysis software, BeadStudio. A bead pool manifest created from the LIMS database containing all the BeadChip data was loaded into BeadStudio along with the intensity data for the samples. BeadStudio used a normalization algorithm to minimize BeadChip to BeadChip variability. Once the normalization was complete, the clustering algorithm was run to evaluate cluster positions for each locus and assign individual genotypes. Each locus was given an overall score based on the quality of the clustering and each individual genotype call was given a GenCall score. GenCall scores provided a quality metric that ranges from 0 to 1 assigned to every genotype called. GenCall scores were then calculated using information from the clustering of the samples. The location of each genotype relative to its assigned cluster determined its GenCall score.

Sample Quality Control:

Quality control measures were intended to identify the samples with the greatest probability of successful CNV identification and to remove the samples with features making CNV identification problematic. Most of the QC metrics employed were originally designed for applications involving high-density genome-wide data. For this study, it was deemed possible that an otherwise high-quality sample with a few large CNVs might fail some QC metrics due to the sparse nature of the data from the custom array employed. The QC process was therefore approached with caution, and inclusion criteria were determined by manual review of the data for each metric in order to identify the outlier values.

Derivative Log Ratio Spread (DLRS):

Derivative Log Ratio Spread (DLRS) is a measurement of point-to-point consistency of LR data, and is a reflection of the signal-to-noise ratio. It is similar in nature to the standard deviation of LR values that is often used in CNV studies, but has the advantage of being robust against large CNVs, which may influence standard deviation. DLRS was calculated for each chromosome, and the median chromosome DLRS value was used as a quality test. The distribution of the median DLRS statistic can be seen below. The outlier threshold was set at 0.3. One hundred twenty-eight subjects fail at this threshold, including all of the 75 samples that failed the waviness factor QC metric (see below).

Waviness Factor:

The “waviness” of each sample in the study was measured using the method of Diskin, et al. [27] as employed within SVS. An absolute value of 0.2 was determined as the outlier threshold for this metric, and 75 subjects failed at this threshold.

Chromosomal Abnormalities and Cell-Line Artifacts:

Fifty-one samples (12 cases and 39 controls) were determined to have a chromosome 21 trisomy, consistent with a diagnosis of Down syndrome. These subjects were later confirmed to have Down syndrome based on clinical data review, and were removed from all further analyses. Additionally, 10 samples were removed based on other abnormalities that appeared to affect entire chromosomes.

Excessive CNVs:

During the course of our analysis, several subjects were noted, using heat map style plots, to have a high frequency of copy number variant regions, in particular copy number gains. To identify the problematic subjects, we estimated the proportion of autosomal CNV regions in the data for which each subject had any CNV gain or loss. After manual review of the distribution of this proportion, 17 subjects with CNV calls at more than 10% of the regions were dropped from further analysis.

Principle Component Analysis (PCA).

Substantial stratification was observed in the LR intensity data. The first two components were stratified by gender, and additional stratification and clustering was observed in the higher components as well. It was therefore considered prudent to apply a PCA correction to the intensity data prior to analysis in order to reduce the probability of data artifacts influencing CNV calls. The principal components were calculated based on all 9,000 samples in the QC process and the results were skewed by the presence of low quality samples. The principle components were therefore recalculated for the 8,777 samples passing preliminary QC, including samples that passed the tests for waviness, DLRS, PCA outliers, chromosome 21 trisomies, and the initial genotyping lab QC. After calculating the first 50 principal components and examining the distribution of eigenvalues, the LR values were corrected for 20 principal components, which were determined to be sufficient to explain the majority of variability in the data. The corrected LR data was then used for segmentation and CNV identification.

CNV Calling:

The segmentation covariates were reduced to a non-redundant spreadsheet, with columns for each marker position where at least one subject had an intensity shift. The distribution of values for each of these columns then was analyzed to determine if multiple copy number states were present, and if so, to estimate the threshold values that defined the different classes. The threshold values were first estimated by a simple algorithm that identified the mode of the distribution, and assuming this to be the neutral copy number state, set upper and lower thresholds based on the variance of the distribution. These thresholds were then manually reviewed, and gross errors were corrected as necessary. After threshold values were confirmed for each of the non-redundant regions, each subject's data for that region was classified accordingly as loss, gain, or neutral. These values were then used to populate a table of discrete copy number calls for use in association testing.

TaqMan Assays:

DNA samples and controls were transferred from stock tubes and diluted with molecular grade water to a final concentration of 5 ng/ul into 0.75 mL Thermo Scientific Matrix storage tubes. All pipetting steps were carried out using Beckman Coulter Biomek FXp automation (Beckman Coulter, Inc., Fullerton, Calif., USA) unless otherwise stated. For each assay, 14 ul of each sample were plated into rows of a 96-well full-skirted plate. The last well in each row was left blank as a non-template control. Each quadrant of the 384-well reaction plates was stamped with 2 ul of DNA from the 96-well sample plate, so that each sample was assayed in quadruplicate. The reaction plates were dried and stored at 4° C. The TaqMan® reaction mix for each assay was prepared according to Applied Biosystems' (Applied Biosystems, Foster City, Calif., USA) recommendations with RNaseP as the reference assay (reference gene) and transferred by hand to each row of a 96-well full-skirted plate. 10 uL of each assay mix was then stamped into the appropriate reaction plate containing 10 ng of dried down DNA per well. The reaction plates were sealed with optical adhesive film, mixed on a plate vortex mixer, and centrifuged prior to running on the Applied Biosystems 7900HT Real Time PCR instrument. Thermal cycling was performed according to the manufacturer's recommended protocol (Applied Biosystems. Data were analyzed with SDS v2.4 software (Applied Biosystems). The baseline was calculated automatically and the threshold was set manually based on the exponential phase of the amplification plot. Data were exported as a text file and imported into the Applied Biosystems CopyCaller v2.0 Program. Assays were analyzed by setting a negative control sample (selected from samples showing none of the CNVs under study by either PennCNV or CNAM) copy number to n=2 except for X chromosome assays, which were analyzed using n=1. For X chromosome CNVs both male and female control samples were used (3 male, 2 female). All other parameters were left as default.

Pathway Analysis.

Ninety of the genes analyzed were within CNV duplications and 63 genes were within CNV deletions. Eighty-seven genes were included since they were the gene nearest to a validated intergenic CNV. Gene abbreviations were batch converted to their Entrez Gene IDs using G:CONVERT [31,32]. Both DAVID and Ingenuity IPA use the right-tailed Fisher's Exact test to calculate P-values representing the probability that selecting genes associated with that pathway or network is due to chance alone.

Network Generation Using IPA:

Each gene in our list of 240 was mapped to its corresponding object in Ingenuity's Knowledge Base. These genes were overlaid onto a global molecular network developed from information contained in Ingenuity's Knowledge Base. Networks then were algorithmically generated based on their connectivity. Both direct and indirect interactions were searched. Network scores are the −log P for the results of a right-tailed Fisher's Exact Test.

Principle Component Analysis (PCA) Results.

Principal components analysis was used to assess the impact of population stratification within the study subjects. Principal components were calculated in SVS using default settings. All subjects were included in the calculation except those that failed data QC. Prior to calculating principal components, the SNPs were filtered so that only SNPs that met the following criteria were used: 1) autosomal SNPs only; 2) call rate>0.95; 3) MAF>0.05; 4) linkage disequilibrium R²<25% for all pairs of SNPs within a moving window of 50 SNPs. In total 2008 SNPs met these criteria. Self-reported ethnicity was used to group samples into “Caucasian” and “non-Caucasian” sets. A simple outlier detection algorithm was applied to stratify the subjects into the two groups. This was done by first calculating the Cartesian distance of each subject from the median centroid of the first two principal component vectors. After determining the third quartile (Q3) and inter-quartile range (IQR) of the distances, any subject with a distance exceeding Q3+1.5*IQR was determined to be outside of the main cluster, and therefore non-Caucasian. Five hundred sixty-four subjects were placed in the non-Caucasian category, including 207 cases and 57 controls. A small number of samples were removed due to duplicate enrollment in the study, but no other unexpected relationships were identified.

TABLE 7
TaqMan Assays Used for CNV Validation

| | Start Coord. | End Coord. | |
|---|---|---|---|
| Chromosome | (hg19) | (hg19) | Assay Name |
| chr1 | 145608130 | 145608131 | Hs01960835_cn |
| chr1 | 145714157 | 145714158 | Hs03356306 |
| chr1 | 145727743 | 145727744 | Hs02151880 |
| chr1 | 145831706 | 145831707 | Hs03363224_cn |
| chr1 | 215857628 | 215857629 | Hs06533545_cn |
| chr1 | 215860518 | 215860519 | Hs05788384_cn |
| chr2 | 13206303 | 13206304 | Hs05832292_cn |
| chr2 | 51257082 | 51257083 | Hs04675592_cn |
| chr2 | 51273782 | 51273783 | Hs03406712_cn |
| chr2 | 51335043 | 51335044 | Hs03207855_cn |
| chr2 | 78417269 | 78417270 | Hs03210777 |
| chr2 | 78448009 | 78448010 | Hs03219183 |
| chr3 | 1940242 | 1940243 | Hs03449476_cn |
| chr3 | 74559838 | 74559839 | Hs06657187_cn |
| chr3 | 74570239 | 74570240 | Hs03006662_cn |
| chr3 | 74580064 | 74580065 | Hs06656853_cn |
| chr3 | 172593661 | 172593662 | Hs05888850_cn |
| chr3 | 172600469 | 172600470 | Hs04760981_cn |
| chr3 | 174853869 | 174853870 | Hs03492315_cn |
| chr3 | 174889051 | 174889052 | Hs03463132_cn |
| chr3 | 176765106 | 176765107 | Hs00705847 |
| chr3 | 176773900 | 176773901 | Hs06653638 |
| chr3 | 178962631 | 178962632 | Hs04718548_cn |
| chr3 | 178969356 | 178969357 | Hs00989875_cn |
| chr4 | 73785471 | 73785472 | Hs04844255_cn |
| chr4 | 73923259 | 73923260 | Hs02916212_cn |
| chr4 | 74027025 | 74027026 | Hs00308217_cn |
| chr4 | 189089063 | 189089064 | Hs03238737 |
| chr4 | 189109145 | 189109146 | Hs03244159 |
| chr5 | 99647650 | 99647651 | Hs03245981_cn |
| chr5 | 99665469 | 99665470 | Hs03248003_cn |
| chr5 | 118544341 | 118544342 | Hs06046822_cn |
| chr5 | 118567989 | 118567990 | Hs03578408_cn |
| chr5 | 118606921 | 118606922 | Hs03562094_cn |
| chr6 | 7464166 | 7464167 | Hs03258806_cn |
| chr6 | 7467367 | 7467368 | Hs03261355_cn |
| chr6 | 39070306 | 39070307 | Hs06797005_cn |
| chr6 | 44131202 | 44131203 | Hs06765368_cn |
| chr6 | 49257472 | 49257473 | Hs06135362_cn |
| chr6 | 62432331 | 62432332 | Hs06740361_cn |
| chr6 | 62468865 | 62468866 | Hs06752297_cn |
| chr6 | 127449047 | 127449048 | Hs04898996 |
| chr6 | 127467261 | 127467262 | Hs06149095 |
| chr6 | 147599263 | 147599264 | Hs00462911_cn |
| chr6 | 147649513 | 147649514 | Hs06799063_cn |
| chr6 | 147681914 | 147681915 | Hs04903013_cn |
| chr7 | 6870706 | 6870707 | Hs03632408_cn |
| chr7 | 15383278 | 15383279 | CusTaq1CX6RM14_cn |
| chr7 | 15405201 | 15405202 | ContR26CX0IV8W_cn |
| chr7 | 93080844 | 93080845 | Hs04974410_cn |
| chr7 | 93145475 | 93145476 | Hs04971099_cn |
| chr7 | 93152478 | 93152479 | Hs04944233_cn |
| chr7 | 100232257 | 100232258 | Hs03629609 |
| chr7 | 100304948 | 100304949 | Hs01981045 |
| chr7 | 100381692 | 100381693 | Hs05013769 |
| chr7 | 124527535 | 124527536 | Hs03620793_cn |
| chr7 | 124578724 | 124578725 | Hs03650226_cn |
| chr7 | 149504056 | 149504057 | Hs03630536 |
| chr7 | 149528561 | 149528562 | Hs03645125 |
| chr7 | 149550437 | 149550438 | Hs03640597 |
| chr8 | 3165293 | 3165294 | Hs02622320_cn |
| chr8 | 54865516 | 54865517 | Hs03668894_cn |
| chr8 | 54905347 | 54905348 | Hs03694907_cn |
| chr8 | 84323860 | 84323861 | Hs04360657 |
| chr8 | 84331501 | 84331502 | Hs03658852 |
| chr8 | 85298919 | 85298920 | Hs03668441_cn |
| chr8 | 85303238 | 85303239 | Hs03678663_cn |
| chr8 | 86467253 | 86467254 | Hs03673176_cn |
| chr9 | 28203352 | 28203353 | Hs03707922_cn |
| chr9 | 28266812 | 28266813 | Hs03714527_cn |
| chr9 | 28333835 | 28333836 | Hs03725541_cn |
| chr9 | 28354528 | 28354529 | Hs03723870_cn |
| chr9 | 136523906 | 136523907 | Hs01617069_cn |
| chr9 | 136527743 | 136527744 | Hs06869845_cn |
| chr9 | 139091261 | 139091262 | Hs06889516_cn |
| chr9 | 139101729 | 139101730 | Hs06847090 |
| chr9 | 139110612 | 139110613 | Hs00495475 |
| chr10 | 83887149 | 83887150 | Hs03726621_cn |
| chr10 | 89717970 | 89717971 | Hs05212456 |
| chr10 | 92274027 | 92274028 | Hs03746257 |
| chr10 | 92287873 | 92287874 | Hs03740287 |
| chr12 | 53178157 | 53178158 | Hs06965067_cn |
| chr12 | 53181253 | 53181254 | Hs06930722_cn |
| chr12 | 71934616 | 71934617 | Hs06933395_cn |
| chr12 | 71950419 | 71950420 | Hs01107784_cn |
| chr12 | 73071721 | 73071722 | Hs06996317_cn |
| chr12 | 73094916 | 73094917 | Hs03093848_cn |
| chr12 | 80898972 | 80898973 | Hs03825941_cn |
| chr12 | 80974071 | 80974072 | Hs03820308_cn |
| chr12 | 81007496 | 81007497 | Hs03818167_cn |
| chr12 | 81610738 | 81610739 | Hs00229436_cn |
| chr12 | 81693094 | 81693095 | Hs00586334_cn |
| chr12 | 81746602 | 81746603 | Hs06985491_cn |
| chr12 | 102097529 | 102097530 | Hs06981209_cn |
| chr12 | 102105668 | 102105669 | Hs04412303_cn |
| chr13 | 40089549 | 40089550 | Hs03853267_cn |
| chr13 | 93444276 | 93444277 | Hs04432382 |
| chr13 | 93460071 | 93460072 | Hs04432043 |
| chr14 | 24519089 | 24519090 | Hs03883350 |
| chr14 | 24534221 | 24534222 | Hs01939905 |
| chr14 | 28522635 | 28522636 | CusTaq2CXLJH4P_cn |
| chr14 | 37916895 | 37916896 | Hs07055190_cn |
| chr14 | 37977977 | 37977978 | Hs07044926_cn |
| chr14 | 38014166 | 38014167 | Hs07086625_cn |
| chr14 | 38021288 | 38021289 | Hs07075472_cn |
| chr14 | 96763309 | 96763310 | Hs05318569_cn |
| chr14 | 96772014 | 96772015 | Hs00982344_cn |
| chr14 | 99641385 | 99641386 | Hs00596122_cn |
| chr14 | 100734909 | 100734910 | Hs03875129 |
| chr14 | 100765197 | 100765198 | Hs01931607 |
| chr14 | 100795059 | 100795060 | Hs00201515 |
| chr14 | 101000582 | 101000583 | Hs03874127_cn |
| chr14 | 101005643 | 101005644 | Hs01983727_cn |
| chr14 | 102021598 | 102021599 | Hs03877829_cn |
| chr14 | 102025461 | 102025462 | Hs03890390_cn |
| chr14 | 102737644 | 102737645 | Hs04443274_cn |
| chr14 | 102744822 | 102744823 | Hs04436664_cn |
| chr14 | 102974514 | 102974515 | Hs03874565_cn |
| chr14 | 104035624 | 104035625 | Hs07076467 |
| chr14 | 104089093 | 104089094 | Hs07094555 |
| chr14 | 104134199 | 104134200 | Hs07101222 |
| chr15 | 20194087 | 20194088 | Hs04444017 |
| chr15 | 25578159 | 25578160 | Hs03899505_cn |
| chr15 | 25580751 | 25580752 | CusTaq3CX20SJR_cn |
| chr15 | 25739587 | 25739588 | Hs03895201_cn |
| chr15 | 26170697 | 26170698 | Hs03899220_cn |
| chr15 | 26218978 | 26218979 | Hs07535627_cn |
| chr15 | 26566910 | 26566911 | Hs05379477_cn |
| chr15 | 26758634 | 26758635 | Hs05357961_cn |
| chr15 | 27186676 | 27186677 | Hs05354636_cn |
| chr15 | 27215751 | 27215752 | Hs05352889_cn |
| chr15 | 28430324 | 28430325 | Hs03904620_cn |
| chr15 | 28464592 | 28464593 | Hs03900299_cn |
| chr15 | 28510861 | 28510862 | Hs00790698_cn |
| chr15 | 30008107 | 30008108 | Hs03905821_cn |
| chr15 | 30028029 | 30028030 | Hs03894282_cn |
| chr15 | 31233791 | 31233792 | Hs01761674_cn |
| chr15 | 31418708 | 31418709 | Hs03907602_cn |
| chr15 | 31523604 | 31523605 | Hs05345027_cn |
| chr15 | 31779480 | 31779481 | Hs01740084_cn |
| chr15 | 31792000 | 31792001 | Hs03903842 |
| chr15 | 31807369 | 31807370 | Hs03898720 |
| chr15 | 31819397 | 31819398 | Hs01183107_cn |
| chr15 | 40565562 | 40565563 | Hs01801490_cn |
| chr15 | 40569495 | 40569496 | Hs03050146_cn |
| chr15 | 40574016 | 40574017 | Hs03915257 |
| chr15 | 40600033 | 40600034 | Hs02747689 |
| chr15 | 40631492 | 40631493 | Hs05348776 |
| chr15 | 42140352 | 42140353 | Hs01736986_cn |
| chr15 | 42220283 | 42220284 | Hs05327333_cn |
| chr15 | 42278083 | 42278084 | Hs07457532_cn |
| chr15 | 56246674 | 56246675 | Hs05388304_cn |
| chr15 | 56258673 | 56258674 | Hs02776763_cn |
| chr16 | 2137638 | 2137639 | Hs03948922_cn |
| chr16 | 2139578 | 2139579 | Hs01690407_cn |
| chr16 | 83908973 | 83908974 | Hs03924139_cn |
| chr16 | 83927884 | 83927885 | Hs03920294_cn |
| chr17 | 14133533 | 14133534 | Hs05489546_cn |
| chr17 | 15285417 | 15285418 | Hs05479141_cn |
| chr19 | 23823676 | 23823677 | Hs07158898_cn |
| chr19 | 23847358 | 23847359 | Hs07130588_cn |
| chr19 | 43260846 | 43260847 | Hs04483050_cn |
| chr19 | 52919934 | 52919935 | Hs01762991_cn |
| chr19 | 52961357 | 52961358 | Hs04015789_cn |
| chr20 | 8654182 | 8654183 | Hs07182273_cn |
| chr20 | 8655323 | 8655324 | Hs07214628_cn |
| chr20 | 8656129 | 8656130 | Hs07196671 |
| chr20 | 8662295 | 8662296 | Hs07181996 |
| chr20 | 32267585 | 32267586 | Hs03035919 |
| chr20 | 32324773 | 32324774 | Hs04040566 |
| chr20 | 32380921 | 32380922 | Hs07167677 |
| chr20 | 35244629 | 35244630 | Hs07189989_cn |
| chr20 | 35286976 | 35286977 | Hs07187468 |
| chr20 | 35339976 | 35339977 | Hs07195828 |
| chr20 | 35392781 | 35392782 | Hs07216584 |
| chr20 | 57246270 | 57246271 | Hs00451592_cn |
| chr20 | 57276159 | 57276160 | Hs02247879_cn |
| chr20 | 57283659 | 57283660 | Hs07195366_cn |
| chrX | 140316814 | 140316815 | Hs04119700_cn |
| chrX | 140348402 | 140348403 | Hs04105155_cn |
| chrX | 140394910 | 140394911 | Hs04123806_cn |
| chrX | 140450224 | 140450225 | Hs04514589_cn |
| chrX | 140560608 | 140560609 | Hs04117605_cn |
| chrX | 140711967 | 140711968 | Hs04108237 |
| chrX | 140730389 | 140730390 | Hs04114029 |
| chrX | 147283785 | 147283786 | Hs05619718 |
| chrX | 147557625 | 147557626 | Hs05666138 |
| chrX | 147831902 | 147831903 | Hs05592380 |
| chrX | 148101715 | 148101716 | Hs05606186 |
| chrX | 148379988 | 148379989 | Hs05667154 |
| chrX | 148892085 | 148892086 | Hs04109160_cn |
| chrX | 148999489 | 148999490 | Hs04513800_cn |
| chrX | 149014384 | 149014385 | Hs02798232_cn |
| chrX | 153195418 | 153195419 | Hs02879994_cn |
| chrX | 153200970 | 153200971 | Hs01730847_cn |

TABLE 8
153 CNVs in subjects with autism in Utah families

| | | | | | | | | | Custom |
|---|---|---|---|---|---|---|---|---|---|
| | | | | | | | | | iSelect |
| | | | | ACRD | | Gain/ | | | Array |
| No. | Chrom | Start (hg19) | End (hg19) | Published? | Ref. No. | Loss | Size (bp) | Gene | Probes |
| 1 | chr1 | 4737693 | 4746636 | N | | Loss | 8943 | AJAP1 | 20 |
| 2 | chr1 | 10624023 | 10627542 | N | | Loss | 3519 | PEX14 | 14 |
| 3 | chr1 | 145714421 | 146101228 | N | | Gain | 386807 | more than 10 genes | 20 |
| 4 | chr1 | 169704308 | 169732211 | N | | Loss | 27903 | C1orf112 | 20 |
| 5 | chr1 | 179456385 | 179472635 | N | | Loss | 16250 | C1orf125/DKFZp434N1720 | 20 |
| 6 | chr1 | 204193679 | 204209979 | N | | Loss | 16300 | PLEKHA6 | 20 |
| 7 | chr1 | 215858193 | 215861879 | Y | 4 | Loss | 3686 | USH2A | 19 |
| 8 | chr1 | 225508461 | 225511454 | N | | Loss | 2993 | DNAH14 | 14 |
| 9 | chr1 | 228848896 | 228853665 | N | | Loss | 4769 | 5′ of RHOU | 11 |
| 10 | chr1 | 237993724 | 237995299 | N | | Loss | 1575 | RYR2 | 15 |
| 11 | chr1 | 243860912 | 243861049 | N | | Loss | 137 | AKT3 | 10 |
| 12 | chr2 | 12685369 | 12693172 | N | | Loss | 7803 | AK001558 | 16 |
| 13 | chr2 | 32982548 | 33050816 | Y | 2, 5 | Gain | 68268 | TTC27, AK095182 | 15 |
| 14 | chr2 | 37904904 | 37909117 | N | | Gain | 4213 | 5′ of CDC42EP3 | 19 |
| 15 | chr2 | 45997209 | 45997519 | N | | Loss | 310 | PRKCE | 11 |
| 16* | chr2 | 51272055 | 51336043 | Y | 2, 4 | Loss | 63988 | 5′ of NRXN1 (10 kb) | 83 |
| 17 | chr2 | 52420563 | 52584090 | N | | Loss | 163527 | 5′ of NRXN1 (1 Mb) | 20 |
| 18 | chr2 | 58346718 | 58349248 | Y | 2 | Loss | 2530 | VRK2 | 12 |
| 19 | chr2 | 62195814 | 62230970 | N | | Loss | 35156 | COMMD1, CR603473 | 20 |
| 20 | chr2 | 75014711 | 75044204 | N | | Loss | 29493 | 5′ of HK2 | 20 |
| 21 | chr2 | 79330766 | 79342811 | N | | Gain | 12045 | 5′ of REG1B, 5′ of | 17 |
| | | | | | | | | REG1A | |
| 22 | chr2 | 120130796 | 120145728 | N | | Loss | 14932 | 5′ of C2orf76, 5′ of | 20 |
| | | | | | | | | TMEM37 | |
| 23 | chr2 | 236424336 | 236465062 | N | | Loss | 40726 | AGAP1 | 20 |
| 24 | chr3 | 6724453 | 7046515 | N | | Gain | 322062 | AF279782, GRM7 | 20 |
| 25 | chr3 | 12387768 | 12393125 | N | | Loss | 5357 | PPARG | 20 |
| 26* | chr3 | 21731567 | 21734331 | N | | Gain | 2764 | ZNF385D | 14 |
| 27 | chr3 | 57051604 | 57053353 | N | | Gain | 1749 | ARHGEF3 | 13 |
| 28 | chr3 | 60774451 | 60777932 | Y | 3 | Gain | 3481 | FHIT | 16 |
| 29 | chr3 | 63962828 | 63964474 | N | | Loss | 1646 | ATXN7 | 13 |
| 30 | chr3 | 74566042 | 74584605 | N | | Loss | 18563 | CNTN3 | 20 |
| 31 | chr3 | 171090367 | 171092891 | N | | Gain | 2524 | TNIK | 16 |
| 32 | chr3 | 172596081 | 172617355 | N | | Gain | 21274 | SPATA16 | 20 |
| 33 | chr4 | 58811798 | 58816810 | N | | Loss | 5012 | 3′ of BC034799 (480 kb) | 14 |
| 34 | chr4 | 80865807 | 80887173 | N | | Loss | 21366 | ANTXR2/DKFZp667K1925 | 17 |
| 35 | chr4 | 101551216 | 101616281 | N | | Loss | 65065 | 5′ of EMCN (200 kb) | 20 |
| 36 | chr4 | 134924034 | 135188390 | N | | Loss | 264356 | PABPC4L | 20 |
| 37 | chr4 | 185734577 | 185740215 | N | | Loss | 5638 | ACSL1 | 18 |
| 38 | chr4 | 189084983 | 189117429 | N | | Loss | 32446 | 3′ of TRIML1 | 20 |
| 39 | chr5 | 20436884 | 20449034 | N | | Loss | 12150 | CDH18 | 20 |
| 40 | chr5 | 58469036 | 58470270 | N | | Loss | 1234 | PDE4D | 12 |
| 41 | chr5 | 99634772 | 99682698 | N | | Loss | 47926 | 5′ of FAM174A (190 kb) | 20 |
| 42 | chr5 | 132621489 | 132630849 | Y | 2, 4 | Gain | 9360 | FSTL4 | 20 |
| 43 | chr5 | 142599442 | 142602063 | N | | Loss | 2621 | ARHGAP26/KIAA0621 | 14 |
| 44 | chr5 | 151582812 | 151583410 | N | | Loss | 598 | AK001582 | 12 |
| 45 | chr6 | 7425246 | 7464367 | N | | Gain | 39121 | 3′ of RIOK1 | 20 |
| 46 | chr6 | 10856101 | 10872458 | N | | Loss | 16357 | 3′ of TMEM14B and | 20 |
| | | | | | | | | GCM2, 5′ of MAK and | |
| | | | | | | | | SYCP2L | |
| 47 | chr6 | 42126761 | 42128299 | N | | Loss | 1538 | GUCA1A | 16 |
| 48 | chr6 | 44113916 | 44180221 | N | | Loss | 66305 | CAPN11, TMEM63B | 20 |
| 49 | chr6 | 47864831 | 49244526 | N | | Loss | 1379695 | C6orf138 | 25 |
| 50 | chr6 | 53856580 | 53864523 | N | | Loss | 7943 | AK056584 | 19 |
| 51 | chr6 | 62443739 | 62462295 | N | | Loss | 18556 | KHDRBS2 | 17 |
| 52 | chr6 | 119419595 | 119427038 | Y | 2 | Loss | 7443 | FAM184A | 18 |
| 53 | chr6 | 123893763 | 123897553 | N | | Loss | 3790 | TRDN | 14 |
| 54 | chr6 | 139985775 | 140128887 | N | | Gain | 143112 | BC039503 | 20 |
| 55 | chr6 | 147588752 | 147664671 | Y | 2 | Gain | 75919 | STXBP5 | 20 |
| 56 | chr6 | 161189018 | 161218651 | N | | Loss | 29633 | 3′ of PLG | 20 |
| 57 | chr7 | 6838712 | 6864071 | N | | Loss | 25359 | C7orf28B | 15 |
| 58 | chr7 | 11782637 | 11783917 | Y | 4 | Loss | 1280 | THSD7A | 12 |
| 59 | chr7 | 13962113 | 13962620 | Y | 2 | Loss | 507 | ETV1 | 11 |
| 60 | chr7 | 71597328 | 71603027 | N | | Gain | 5699 | CALM | 14 |
| 61 | chr7 | 105285949 | 105321353 | N | | Loss | 35404 | ATXN7L1 | 20 |
| 62 | chr7 | 124546250 | 124580202 | Y | 4 | Loss | 33952 | POT1, hypothetical proteins | 20 |
| 63 | chr8 | 3160739 | 3160885 | N | | Loss | 146 | CSMD1/KIAA1890 | 10 |
| 64 | chr8 | 3169351 | 3169808 | N | | Loss | 457 | CSMD1/KIAA1890 | 11 |
| 65 | chr8 | 3479586 | 3480400 | N | | Loss | 814 | CSMD1 | 12 |
| 66 | chr8 | 4907673 | 4911422 | N | | Loss | 3749 | 5′ of CSMD1 60 kb) | 20 |
| 67 | chr8 | 31977229 | 31989597 | N | | Loss | 12368 | NRG1 | 20 |
| 68 | chr8 | 52261992 | 52265315 | N | | Loss | 3323 | PXDNL | 15 |
| 69 | chr8 | 84323466 | 84337983 | N | | Loss | 14517 | 3′ of BC038578 | 20 |
| 70 | chr8 | 85281895 | 85304198 | N | | Loss | 22303 | RALYL | 20 |
| 71 | chr8 | 86471729 | 86553130 | N | | Gain | 81401 | 3′ of REXO1L1 | 20 |
| 72 | chr8 | 100402969 | 100406592 | N | | Loss | 3623 | VPS13B | 10 |
| 73 | chr9 | 7036350 | 7051859 | N | | Loss | 15509 | JMJD2C | 20 |
| 74 | chr9 | 28027694 | 28039222 | N | | Gain | 11528 | LINGO2 | 20 |
| 75 | chr9 | 28190069 | 28347679 | N | | Loss | 157610 | LINGO2 | 20 |
| 76 | chr9 | 75206337 | 75207666 | N | | Gain | 1329 | TMC1 | 11 |
| 77 | chr9 | 116468123 | 116631674 | N | | Gain | 163551 | 5′ of ZNF618 (5 kb) | 12 |
| 78 | chr9 | 139083019 | 139113146 | N | | Gain | 30127 | LHX3, QSOX2 | 20 |
| 79 | chr10 | 27361202 | 27381349 | N | | Loss | 20147 | ANKRD26 | 20 |
| 80 | chr10 | 33217225 | 33222978 | N | | Loss | 5753 | ITGB1 | 11 |
| 81 | chr10 | 38914665 | 42953131 | N | | Loss | 4038466 | AK131313, BC039000 | 20 |
| 82 | chr10 | 52133698 | 52232708 | Y | 3 | Gain | 99010 | SGMS1/SMS1 | 20 |
| 83 | chr10 | 60793303 | 60857532 | Y | 3 | Gain | 64229 | 5′ of PHYHIPL (80 kb) | 20 |
| 84 | chr10 | 68350062 | 68375800 | N | | Loss | 25738 | CTNNA3 | 20 |
| 85 | chr10 | 81032555 | 81037800 | N | | Loss | 5245 | ZMIZ1 | 14 |
| 86 | chr10 | 83893626 | 84175018 | N | | Loss | 281392 | NRG3 | 13 |
| 87 | chr10 | 86939018 | 86970632 | N | | Loss | 31614 | AK097624 | 20 |
| 88 | chr10 | 89720106 | 89723874 | N | | Loss | 3768 | PTEN | 12 |
| 89 | chr10 | 91210650 | 91217984 | N | | Loss | 7334 | SLC16A12 | 19 |
| 90 | chr10 | 92274764 | 92289762 | Y | 2 | Loss | 14998 | 3′ of BC037970 | 15 |
| 91 | chr11 | 7488341 | 7489819 | N | | Gain | 1478 | SYT9, AK128569 | 16 |
| 92 | chr11 | 12002139 | 12007077 | N | | Gain | 4938 | DKK3 | 20 |
| 93 | chr11 | 12374189 | 12374712 | N | | Loss | 523 | MICALCL | 11 |
| 94 | chr11 | 16569019 | 16576640 | N | | Loss | 7621 | SOX6/DKFZp434N1217 | 12 |
| 95 | chr11 | 31000774 | 31000929 | N | | Gain | 155 | DCDC5/KIAA1493 | 10 |
| 96 | chr11 | 60228735 | 60229382 | N | | Loss | 647 | MS4A1 | 11 |
| 97 | chr11 | 98148399 | 98212796 | N | | Gain | 64397 | 5′ of CNTN5 (700 kb) | 20 |
| 98 | chr11 | 100817655 | 100820663 | N | | Loss | 3008 | FLJ32810 | 14 |
| 99 | chr11 | 131405729 | 131406206 | N | | Gain | 477 | NTM, AK128059 | 11 |
| 100 | chr12 | 60173356 | 60173878 | Y | 4 | Gain | 522 | SLC16A7/MCT2 | 13 |
| 101 | chr12 | 73062598 | 73088289 | Y | 2 | Loss | 25691 | 3′ of TRHDE | 20 |
| 102 | chr12 | 75547922 | 75572356 | N | | Loss | 24434 | KCNC2 | 20 |
| 103 | chr12 | 80880491 | 80895554 | N | | Loss | 15063 | PTPRQ | 20 |
| 104 | chr12 | 80988331 | 81019079 | N | | Loss | 30748 | PTPRQ | 20 |
| 105 | chr12 | 81618586 | 81626675 | N | | Loss | 8089 | ACSS3 | 17 |
| 106 | chr12 | 97870273 | 97875696 | N | | Loss | 5423 | NCRMS/AK056164 | 20 |
| 107 | chr12 | 102097012 | 102106306 | N | | Loss | 9294 | CHPT1 | 13 |
| 108 | chr12 | 127308503 | 127315005 | Y, small | 4 | Loss | 6502 | between BC069215 | 19 |
| | | | | overlap | | | | and BC037858 | |
| 109 | chr13 | 40087689 | 40088007 | N | | Loss | 318 | LHFP | 12 |
| 110 | chr13 | 49284461 | 49343043 | N | | Gain | 58582 | 3′ of CYSLTR2 | 20 |
| 111 | chr13 | 50163809 | 50179454 | N | | Loss | 15645 | 5′ of RCBTB1 | 17 |
| 112 | chr13 | 93448487 | 93461603 | N | | Loss | 13116 | GPC5 | 17 |
| 113 | chr13 | 94357235 | 94369759 | N | | Loss | 12524 | GPC6 | 20 |
| 114 | chr14 | 23862374 | 23888040 | N | | Loss | 25666 | MYH6, MYH7, | 20 |
| | | | | | | | | MIR208B | |
| 115 | chr14 | 28506099 | 28520243 | N | | Loss | 14144 | between BC148262 | 20 |
| | | | | | | | | and CR597916 | |
| 116 | chr14 | 32904231 | 32909169 | N | | Gain | 4938 | AKAP6 | 20 |
| 117 | chr14 | 33859159 | 33860185 | N | | Gain | 1026 | NPAS3 | 11 |
| 118 | chr14 | 37928753 | 37948391 | N | | Loss | 19638 | MIPOL1 | 15 |
| 119 | chr14 | 68068610 | 68071772 | N | | Loss | 3162 | 5′ of PIGH | 15 |
| 120 | chr15 | 33605301 | 33617521 | N | | Gain | 12220 | RYR3 | 20 |
| 121 | chr15 | 47518807 | 47527672 | N | | Loss | 8865 | SEMA6D | 16 |
| 122 | chr15 | 58851369 | 58853307 | N | | Gain | 1938 | LIPC | 14 |
| 123 | chr15 | 60074956 | 60103803 | Y | 5 | Loss | 28847 | 5′ of BNIP2 (90 kb) | 20 |
| 124 | chr15 | 66521832 | 66524433 | N | | Loss | 2601 | MEGF11 | 17 |
| 125 | chr15 | 87830530 | 87870489 | N | | Loss | 39959 | between AGBL1, and | 20 |
| | | | | | | | | TMEM83, NTRK3 | |
| 126 | chr16 | 16245729 | 16256767 | N | | Loss | 11038 | ABCC6, MRP6 | 34 |
| 127 | chr16 | 21363810 | 21602618 | N | | Loss | 238808 | More than 10 genes | 25 |
| 128 | chr16 | 82446255 | 82711504 | Y | 5 | Gain | 265249 | CDH13 | 24 |
| 129 | chr16 | 83909041 | 83926368 | N | | Loss | 17327 | 5′ of MLYCD, 3′ of | 20 |
| | | | | | | | | HSBP1 | |
| 130 | chr17 | 4007594 | 4324408 | Y | 4 | Gain | 316814 | ZZEF1, KIAA0399, | 20 |
| | | | | | | | | CYB5D2, ANKFY1, | |
| | | | | | | | | UBE2G1, SPNS3 | |
| 131** | chr17 | 21556170 | 25363654 | N | | Loss | 3807484 | BC070367, FAM27L, | 20 |
| | | | | | | | | BC039120, CR592140, | |
| | | | | | | | | CR592128 | |
| 132 | chr17 | 39211908 | 39221312 | N | | Loss | 9404 | KRTAP2-4 | 15 |
| 133 | chr17 | 64258845 | 64259329 | N | | Loss | 484 | 5′ of APOH and 5′ of | 11 |
| | | | | | | | | PRKCA | |
| 134 | chr18 | 30037470 | 30037675 | N | | Loss | 205 | FAM59A | 10 |
| 135 | chr20 | 4234781 | 4238447 | N | | Gain | 3666 | 5′ of ADRA1D | 16 |
| 136 | chr20 | 6013320 | 6017259 | N | | Loss | 3939 | CRLS1/DKFZp762C112 | 14 |
| 137 | chr20 | 15755244 | 15765167 | N | | Loss | 9923 | MACROD2 | 20 |
| 138 | chr20 | 47337049 | 47341312 | N | | Gain | 4263 | PREX1 | 14 |
| 139 | chr20 | 49132410 | 49132637 | N | | Loss | 227 | PTPN1 | 10 |
| 140 | chr20 | 56248075 | 56252910 | N | | Loss | 4835 | PMEPA1 | 20 |
| 141 | chr21 | 17311697 | 17435462 | N | | Loss | 123765 | 5′ of C21orf34, 3′ of | 20 |
| | | | | | | | | USP25 | |
| 142 | chr21 | 42855515 | 42855647 | Y | 1 | Gain | 132 | TMPRSS2 | 10 |
| 143 | chr22 | 30731066 | 30731540 | N | | Gain | 474 | SF3A1 | 10 |
| 144 | chr22 | 33459104 | 33470309 | N | | Loss | 11205 | 5′ of SYN3 | 20 |
| 145 | chr22 | 39515118 | 39525791 | N | | Loss | 10673 | 3′ of APOBECSH, 3′ of | 20 |
| | | | | | | | | CBX7 | |
| 146 | chr22 | 44251958 | 44257056 | N | | Loss | 5098 | SULT4A1/SULTX3 | 19 |
| 147 | chr22 | 44641315 | 44641594 | N | | Gain | 279 | KIAA1644 | 10 |
| 148 | chr22 | 51055900 | 51234443 | Y | 4 | Gain | 178543 | ARSA, SHANK3, | 10 |
| | | | | | | | | BC050343, ACR, | |
| | | | | | | | | MGC70863, RABL2B | |
| 149 | chrX | 3206732 | 3216695 | N | | Loss | 9963 | 3′ of MXRA5, ARSF | 19 |
| 150 | chrX | 57285994 | 57291268 | N | | Gain | 5274 | 5′ of FAAH2 | 11 |
| 151 | chrX | 133460586 | 133466162 | N | | Loss | 5576 | 5′ of PHF6 | 11 |
| 152 | chrX | 142769032 | 142781735 | N | | Loss | 12703 | 5′ of SLITRK4, 3′ of | 15 |
| | | | | | | | | SPANXN2 | |
| 153 | chrX | 151041009 | 151042244 | N | | Loss | 1235 | 5′ of MAGEA4 | 12 |
| | | | | | | | | | Total = |
| | | | | | | | | | 2,642 |
| | | | | | | | | | Probes |

References:
1. Jacquemont et al., 2006
2. AGP, 2007
3. Sebat et al., 2007
4. Marshall et al., 2008
5. Christian et al., 2008
*Nos 16 & 26: includes overlapping literature CNVs
**No. 131: Much of this region spans the centromere and is heterochromatic

TABLE 9
185 CNVs reportedly associated with ASD from published studies

| | | | Custom |
|---|---|---|---|
| | | CNV Origin | iSelect |
| | | CHOP | Array |
| No. | CNV Regions (hg19, GRCh37) | Literature | Probes |
| 1 | chr1: 146626687-146641912 | CHOP_CNV | 208 |
| 2 | chr1: 146644352-146646782 | CHOP_CNV | 208 |
| 3 | chr1: 146649431-146651526 | CHOP_CNV | 208 |
| 4 | chr1: 146655885-146661221 | CHOP_CNV | 208 |
| 5 | chr1: 146714336-146767441 | CHOP_CNV | 208 |
| 6 | chr1: 147013183-147042947 | CHOP_CNV | 208 |
| 7 | chr1: 147119170-147142612 | CHOP_CNV | 208 |
| 8 | chr1: 147191843-147211176 | CHOP_CNV | 208 |
| 9 | chr1: 147228333-147245482 | CHOP_CNV | 208 |
| 10 | chr1: 152538131-152539246 | CHOP_CNV | 22 |
| 11 | chr1: 152551861-152552978 | CHOP_CNV | 22 |
| 12 | chr1: 176233934-176277050 | CHOP_CNV | 20 |
| 13 | chr2: 13202218-13248445 | CHOP_CNV | 20 |
| 14 | chr2: 37208154-37311483 | CHOP_CNV | 20 |
| 15 | chr2: 50147489-51240182 | CHOP_CNV | 84 |
| 16 | chr2: 51267143-51294094 | CHOP_CNV | 62 |
| 17 | chr2: 78414693-78457739 | CHOP_CNV | 20 |
| 18 | chr2: 99858712-99871568 | CHOP_CNV | 17 |
| 19 | chr2: 237821591-237832364 | CHOP_CNV | 94 |
| 20 | chr3: 1940192-1940920 | CHOP_CNV | 10 |
| 21 | chr3: 2573150-2573529 | CHOP_CNV | 11 |
| 22 | chr3: 4224733-4261302 | CHOP_CNV | 20 |
| 23 | chr3: 31702318-32023236 | CHOP_CNV | 20 |
| 24 | chr3: 37903670-38025958 | CHOP_CNV | 20 |
| 25 | chr3: 121343502-121387782 | CHOP_CNV | 20 |
| 26 | chr3: 172231370-173116242 | CHOP_CNV | 116 |
| 27 | chr3: 173116245-173254086 | CHOP_CNV | 100 |
| 28 | chr3: 173271686-173289279 | CHOP_CNV | 100 |
| 29 | chr3: 174001117-174885989 | CHOP_CNV | 100 |
| 30 | chr4: 13656804-13932850 | CHOP_CNV | 20 |
| 31 | chr4: 73756500-73905356 | CHOP_CNV | 60 |
| 32 | chr4: 73920417-73935470 | CHOP_CNV | 60 |
| 33 | chr4: 73940504-74124500 | CHOP_CNV | 60 |
| 34 | chr4: 144627954-144635127 | CHOP_CNV | 11 |
| 35 | chr5: 118229547-118343923 | CHOP_CNV | 100 |
| 36 | chr5: 118407187-118469872 | CHOP_CNV | 100 |
| 37 | chr5: 118478541-118584821 | CHOP_CNV | 100 |
| 38 | chr5: 118604420-118730292 | CHOP_CNV | 100 |
| 39 | chr5: 118730295-118856171 | CHOP_CNV | 100 |
| 40 | chr6: 39071841-39082863 | CHOP_CNV | 20 |
| 41 | chr6: 69235102-69237305 | CHOP_CNV | 10 |
| 42 | chr6: 122793063-123047516 | CHOP_CNV | 34 |
| 43 | chr6: 127440049-127518908 | CHOP_CNV | 20 |
| 44 | chr6: 135818945-136037191 | CHOP_CNV | 20 |
| 45 | chr6: 162664588-162667009 | CHOP_CNV | 31 |
| 46 | chr6: 168349013-168596249 | CHOP_CNV | 20 |
| 47 | chr7: 2649899-2654358 | CHOP_CNV | 20 |
| 48 | chr7: 32700564-32804186 | CHOP_CNV | 20 |
| 49 | chr7: 69064321-70257852 | CHOP_CNV | 23 |
| 50 | chr7: 111502940-111846460 | CHOP_CNV | 20 |
| 51 | chr7: 141695680-141806545 | CHOP_CNV | 20 |
| 52 | chr8: 43646415-43657436 | CHOP_CNV | 20 |
| 53 | chr8: 54858496-54907579 | CHOP_CNV | 20 |
| 54 | chr9: 116111824-116132133 | CHOP_CNV | 86 |
| 55 | chr9: 116135700-116139257 | CHOP_CNV | 85 |
| 56 | chr9: 119187508-120177315 | CHOP_CNV | 58 |
| 57 | chr9: 136501486-136524464 | CHOP_CNV | 37 |
| 58 | chr10: 87359313-87944322 | CHOP_CNV | 105 |
| 59 | chr10: 87951688-87959047 | CHOP_CNV | 79 |
| 60 | chr10: 88126251-88893189 | CHOP_CNV | 104 |
| 61 | chr10: 105353785-105615162 | CHOP_CNV | 20 |
| 62 | chr10: 118350491-118368684 | CHOP_CNV | 20 |
| 63 | chr12: 31409581-31410819 | CHOP_CNV | 13 |
| 64 | chr12: 53183470-53189890 | CHOP_CNV | 20 |
| 65 | chr12: 57345220-57352101 | CHOP_CNV | 20 |
| 66 | chr12: 71833814-71980084 | CHOP_CNV | 20 |
| 67 | chr13: 20977807-21100010 | CHOP_CNV | 20 |
| 68 | chr14: 94184645-94254764 | CHOP_CNV | 20 |
| 69 | chr15: 23686020-23692388 | CHOP_CNV | 19 |
| 70 | chr15: 24842742-24979665 | CHOP_CNV | 47 |
| 71 | chr15: 25101701-25223727 | CHOP_CNV | 53 |
| 72 | chr16: 16243423-16317335 | CHOP_CNV | 40 |
| 73 | chr16: 47276822-47330242 | CHOP_CNV | 20 |
| 74 | chr16: 70954495-71007921 | CHOP_CNV | 20 |
| 75 | chr16: 75572016-75590168 | CHOP_CNV | 20 |
| 76 | chr16: 84599210-84610700 | CHOP_CNV | 40 |
| 77 | chr17: 30819629-31203900 | CHOP_CNV | 20 |
| 78 | chr17: 64298927-64806860 | CHOP_CNV | 31 |
| 79 | chr18: 3498838-3880133 | CHOP_CNV | 20 |
| 80 | chr19: 22639351-22639555 | CHOP_CNV | 10 |
| 81 | chr19: 23835709-23870015 | CHOP_CNV | 38 |
| 82 | chr19: 23926161-23941637 | CHOP_CNV | 38 |
| 83 | chr19: 43225795-43440224 | CHOP_CNV | 20 |
| 84 | chr19: 52880583-52901119 | CHOP_CNV | 108 |
| 85 | chr19: 52901122-52909308 | CHOP_CNV | 108 |
| 86 | chr19: 52909311-52921656 | CHOP_CNV | 108 |
| 87 | chr19: 52932442-52934660 | CHOP_CNV | 108 |
| 88 | chr19: 52934663-52942694 | CHOP_CNV | 108 |
| 89 | chr19: 52956761-52961405 | CHOP_CNV | 108 |
| 90 | chr20: 8113297-8865545 | CHOP_CNV | 40 |
| 91 | chr20: 55993557-55997466 | CHOP_CNV | 33 |
| 92 | chr22: 21021266-21028944 | CHOP_CNV | 19 |
| 93 | chr22: 29999566-30094583 | CHOP_CNV | 20 |
| 94 | chrX: 6966962-7066187 | CHOP_CNV | 20 |
| 95 | chrX: 139998330-140335594 | CHOP_CNV | 71 |
| 96 | chrX: 140335597-140443613 | CHOP_CNV | 71 |
| 97 | chrX: 140590844-140672859 | CHOP_CNV | 71 |
| 98 | chrX: 140677836-140678897 | CHOP_CNV | 71 |
| 99 | chrX: 140713997-140714859 | CHOP_CNV | 71 |
| 100 | chrX: 148663310-148669114 | CHOP_CNV | 60 |
| 101 | chrX: 148676928-148678215 | CHOP_CNV | 60 |
| 102 | chrX: 148678218-148713566 | CHOP_CNV | 60 |
| 103 | chrX: 148858522-149097275 | CHOP_CNV | 60 |
| 104 | chrX: 154719774-154842595 | CHOP_CNV | 40 |
| 105 | chr1: 110230419-110236364 | Literature_CNV | 0 |
| 106 | chr1: 146555186-147779086 | Literature_CNV | 152 |
| 107 | chr1: 162573378-167543374 | Literature_CNV | 61 |
| 108 | chr1: 230111830-232145817 | Literature_CNV | 43 |
| 109 | chr2: 54076-1198908 | Literature_CNV | 23 |
| 110 | chr2: 17406571-18378433 | Literature_CNV | 21 |
| 111 | chr2: 32678416-33378738 | Literature_CNV | 40 |
| 112 | chr2: 45455651-45984915 | Literature_CNV | 31 |
| 113 | chr2: 50145644-51259671 | Literature_CNV | 84 |
| 114 | chr2: 51979551-52401447 | Literature_CNV | 40 |
| 115 | chr2: 57200002-61699998 | Literature_CNV | 98 |
| 116 | chr2: 62258231-63028717 | Literature_CNV | 48 |
| 117 | chr2: 115139568-115617934 | Literature_CNV | 20 |
| 118 | chr2: 162387215-162840241 | Literature_CNV | 20 |
| 119 | chr2: 198797484-209741388 | Literature_CNV | 119 |
| 120 | chr2: 236632457-238435065 | Literature_CNV | 101 |
| 121 | chr2: 238435068-242985349 | Literature_CNV | 125 |
| 122 | chr3: 2028902-2884398 | Literature_CNV | 31 |
| 123 | chr3: 11034422-11080933 | Literature_CNV | 20 |
| 124 | chr3: 67656832-68957204 | Literature_CNV | 24 |
| 125 | chr3: 100203669-100487283 | Literature_CNV | 20 |
| 126 | chr3: 143608410-144494785 | Literature_CNV | 20 |
| 127 | chr3: 195674002-197284998 | Literature_CNV | 27 |
| 128 | chr4: 154087652-172339893 | Literature_CNV | 191 |
| 129 | chr5: 176990003-180905258 | Literature_CNV | 42 |
| 130 | chr6: 13889303-15153950 | Literature_CNV | 24 |
| 131 | chr7: 23876-1297908 | Literature_CNV | 16 |
| 132 | chr7: 15386880-15538756 | Literature_CNV | 20 |
| 133 | chr7: 72576596-75922729 | Literature_CNV | 42 |
| 134 | chr7: 83144216-86082367 | Literature_CNV | 40 |
| 135 | chr7: 87999366-89294562 | Literature_CNV | 24 |
| 136 | chr7: 121210655-121381762 | Literature_CNV | 40 |
| 137 | chr7: 121755766-122152424 | Literature_CNV | 40 |
| 138 | chr7: 128907065-128998138 | Literature_CNV | 20 |
| 139 | chr7: 152589804-152616097 | Literature_CNV | 20 |
| 140 | chr8: 6264122-6506023 | Literature_CNV | 20 |
| 141 | chr8: 53271330-53555369 | Literature_CNV | 20 |
| 142 | chr9: 7735282-7770231 | Literature_CNV | 20 |
| 143 | chr9: 38027602-38298598 | Literature_CNV | 20 |
| 144 | chr9: 102472181-136065177 | Literature_CNV | 464 |
| 145 | chr10: 13049365-13367445 | Literature_CNV | 20 |
| 146 | chr10: 46269076-50892143 | Literature_CNV | 64 |
| 147 | chr10: 50892146-51450787 | Literature_CNV | 32 |
| 148 | chr10: 84158614-89685463 | Literature_CNV | 178 |
| 149 | chr11: 40329226-40653822 | Literature_CNV | 20 |
| 150 | chr13: 23604102-24794298 | Literature_CNV | 23 |
| 151 | chr13: 35516457-36246870 | Literature_CNV | 20 |
| 152 | chr13: 48083039-48475962 | Literature_CNV | 20 |
| 153 | chr13: 67572852-67762297 | Literature_CNV | 20 |
| 154 | chr15: 20266959-25480660 | Literature_CNV | 123 |
| 155 | chr15: 25582397-25684125 | Literature_CNV | 28 |
| 156 | chr15: 73090002-76507998 | Literature_CNV | 44 |
| 157 | chr15: 85105976-85708062 | Literature_CNV | 20 |
| 158 | chr16: 2097991-2138710 | Literature_CNV | 20 |
| 159 | chr16: 6052837-6260813 | Literature_CNV | 20 |
| 160 | chr16: 14982501-16482497 | Literature_CNV | 64 |
| 161 | chr16: 21534307-21901307 | Literature_CNV | 48 |
| 162 | chr16: 21901310-22703860 | Literature_CNV | 34 |
| 163 | chr16: 29671216-30173786 | Literature_CNV | 20 |
| 164 | chr16: 82195236-82722082 | Literature_CNV | 40 |
| 165 | chr17: 9964035-10361280 | Literature_CNV | 20 |
| 166 | chr17: 14139846-15282723 | Literature_CNV | 23 |
| 167 | chr17: 48646233-48704540 | Literature_CNV | 20 |
| 168 | chr18: 32073255-35145997 | Literature_CNV | 42 |
| 169 | chr19: 27896698-28805250 | Literature_CNV | 20 |
| 170 | chr20: 127914-419869 | Literature_CNV | 20 |
| 171 | chr20: 2837196-4006397 | Literature_CNV | 23 |
| 172 | chr20: 8044044-8527513 | Literature_CNV | 30 |
| 173 | chr20: 41602847-41867105 | Literature_CNV | 20 |
| 174 | chr21: 37412682-37622182 | Literature_CNV | 20 |
| 175 | chr22: 18640348-21461644 | Literature_CNV | 51 |
| 176 | chr22: 38368320-38380536 | Literature_CNV | 20 |
| 177 | chr22: 47956883-49122331 | Literature_CNV | 36 |
| 178 | chr22: 49405478-49971756 | Literature_CNV | 29 |
| 179 | chr22: 51113071-51171638 | Literature_CNV | 36 |
| 180 | chrX: 94421-5469456 | Literature_CNV | 78 |
| 181 | chrX: 5808084-5999993 | Literature_CNV | 20 |
| 182 | chrX: 28605682-29974014 | Literature_CNV | 25 |
| 183 | chrX: 53300002-53699998 | Literature_CNV | 20 |
| 184 | chrX: 70364712-70391048 | Literature_CNV | 20 |
| 185 | chrX: 153213010-153399998 | Literature_CNV | 40 |
| | | | Total = |
| | | | 4,492 |
| | | | probes* |

*Note that there is significant redundancy in this probe set, as many of the literature CNVs included on the array overlapped.

TABLE 10
25 CNVs identified from single nucleotide variants (SNVs) on custom array

| | | Gain or | Validation | | Start Coord. | End Coord. | |
|---|---|---|---|---|---|---|---|
| No. | CNV Source | Loss | Status | Chromosome | (hg19) | (hg19) | Gene(s) |
| 1 | SequenceSNP | Loss | PASS | chr7 | 93070811 | 93116320 | CALCR MIR653 MIR489 |
| 2 | SequenceSNP | Gain | PASS | chr14 | 100705631 | 100828134 | SLC25A29 YY1 MIR345 |
| | | | | | | | SLC25A47 WARS |
| 3 | SequenceSNP | Gain | PASS | chr14 | 102018946 | 102026138 | DIO3AS DIO3OS |
| 4 | SequenceSNP | Loss | PASS | chr14 | 102729881 | 102749930 | MOK/RAGE |
| 5 | SequenceSNP | Gain | PASS | chr14 | 102973910 | 102975572 | ANKRD9 |
| 6 | SequenceSNP | Gain | PASS | chr15 | 25690465 | 26793077 | ATP10A MIR4715 |
| | | | | | | | GABRB3 LOC503519 |
| | | | | | | | LOC100128714 |
| 7 | SequenceSNP | Gain | PASS | chr15 | 27184517 | 27216737 | GABRA5 GABRG3 |
| 8 | SequenceSNP | Gain | PASS | chr15 | 28408312 | 28513763 | HERC2 |
| 9 | SequenceSNP | Loss | PASS | chr15 | 31092983 | 31369123 | FAN1 TRPM1 MTMR10 |
| | | | | | | | MIR211 TRPM1 |
| 10 | SequenceSNP | Gain/Loss | PASS | chr15 | 31776648 | 31822910 | OTUD7A |
| 11 | SequenceSNP | Gain | PASS | chr20 | 32210931 | 32441302 | NECAB3 CBFA2T2 E2F1 |
| | | | | | | | C20orf134 ZNF341 |
| | | | | | | | C20orf144 PXMP4 ZNF341 |
| | | | | | | | CHMP4B |
| 12 | SequenceSNP | Gain | No data | chr14 | 99640708 | 99642376 | BCL11B |
| 13 | SequenceSNP | Loss | FAIL | chr3 | 176755900 | 176782811 | TBL1XR1 |
| 14 | SequenceSNP | Gain | FAIL | chr7 | 100159979 | 100456457 | MOSPD3 TFR2 |
| | | | | | | | LOC100129845 GIGYF1 |
| | | | | | | | GNB2 LRCH4 ACTL6B |
| | | | | | | | FBXO24 PCOLCE AGFG2 |
| | | | | | | | SAP25 POP7 GIGF1 ZAN |
| | | | | | | | SLC12A9 EPHB4 |
| 15 | SequenceSNP | Gain/Loss | FAIL | chr7 | 149481075 | 149576256 | SSPO ATP6V0E2 ZNF862 |
| | | | | | | | LOC401431 |
| 16 | SequenceSNP | Gain | FAIL | chr14 | 24507010 | 24550497 | DHRS4L1 LRRC16B NRL |
| | | | | | | | CPNE6 |
| 17 | SequenceSNP | Loss | FAIL | chr14 | 96758018 | 96777946 | ATG2B |
| 18 | SequenceSNP | Gain | FAIL | chr14 | 100995537 | 101010301 | BEGAIN WDR25 |
| 19 | SequenceSNP | Gain | FAIL | chr14 | 103986349 | 104182224 | TRMT61A CKB TRMT61A |
| | | | | | | | BAG5 APOPT1 C14orf153 |
| | | | | | | | XRCC3 KLC1 ZFYVE21 |
| 20 | SequenceSNP | Gain | FAIL | chr15 | 30000877 | 30033536 | TJP1 |
| 21 | SequenceSNP | Gain | FAIL | chr15 | 40544493 | 40661306 | C15orf56 PAK6 PLCB2 |
| | | | | | | | C15orf52 DISP2 |
| 22 | SequenceSNP | Gain | FAIL | chr15 | 42139583 | 42302433 | JMJD7-PLA2G4B |
| | | | | | | | PLA2G4B SPTBN5 EHD4 |
| | | | | | | | PLA2G4E |
| 23 | SequenceSNP | Loss | FAIL | chr15 | 56243611 | 56258744 | NEDD4 |
| 24 | SequenceSNP | Gain | FAIL | chr20 | 35234192 | 35444437 | NDRG3 TGIF2-C20ORF24 |
| | | | | | | | C20orf24 SLA2 DSN1 |
| | | | | | | | KIAA0889 |
| 25 | SequenceSNP | Gain | FAIL | chr20 | 57268867 | 57290347 | NPEPL1 STX16-NPEPL1 |

### Example 2

### Design of a Custom Clinical Array

A custom clinical array was designed based on the results of the study described in Example 1. The study array used in Example 1 included about 10,000 probes for the regions being studied. Therefore, a custom array was specifically designed for clinical use to enhance coverage for the CNVs identified as associated with ASD. Custom probes for detection of other childhood developmental delay disorders were also included on the array as outlined in Table 11 below.

Table 11 below summarizes the custom probes designed for and included on the clinical array. The clinical array is based on the Affymetrix CytoScan-HD array and includes the 83,443 custom probes provided in the accompanying sequence listing. The 83,443 probes were added to the Affymetrix array to ensure sufficient coverage of all of the regions described in Tables 8 and 9, as well as to detect CNVs for the other disorders listed in Table 11.

TABLE 11
Summary of Custom Probes

| | | Custom CNV |
|---|---|---|
| Disorder | CNV source | Probes |
| Autism | Literature CNVs | 58950 |
| | Utah CNVs | 3691 |
| | CHOP CNVs | 2619 |
| Utah familial sequence variants | | |
| Rett syndrome | | 28 |
| Noonan/Costello/CFC syndromes | | 0 |
| Tuberous sclerosis | | 0 |
| ADHD | | 8764 |
| DD | | 9364 |
| Tourette syndrome | | 27 |
| Dyslexia | | 0 |
| | Total | 83443 |

A description of the custom probes as summarized in Table 11 is provided in Table 14 of U.S. Provisional Application 61/977,462 and Table 14 from International PCT Publication No. 2014/055915, the disclosure of each of which is incorporated by reference in their entireties. Table 14 from these disclosures provides the following information: The third column, labeled “hg19 Coordinates/Gene Name”, displays the genome coordinates (hg19) of the CNV for which each probe was designed. The second column, labeled “EXPOS” displays the nucleotide position within the chromosomal region shown in the third column that represents the center of the oligonucleotide probe. The oligonucleotides themselves are 25 nucleotides in length, so the center is nucleotide 13. The first column lists the SEQ ID NO for the oligonucleotide (DNA probe) which is provided in the accompanying sequence listing.

Tables 12 and 13 below list the CNVs identified in the study described in Example 1 (from Tables 3 and 4), and further include the SEQ ID NOs for the custom probes, where applicable. Since custom probes were only included on the array for some CNVs identified in Example 1, N/A is used to denote that no custom probes were used. Sequences of the custom probes are set forth in the sequence listing as SEQ ID NOs:1-83,443. As noted above, the positions of the probes are described in Table 14 of U.S. Provisional Application 61/977,462 and Table 14 of International PCT Publication No. 2014/055915, the disclosure of each of which is incorporated by reference in their entireties.

TABLE 12
Summary of Custom Probes for CNVs from Table 3

| | | | Custom Probe |
|---|---|---|---|
| No. | CNV Region - Replication Cohort | Gene/Region | SEQ ID NOs¹ |
| 1 | chr1: 145703115-145736438 | CD160, PDZK1 | N/A |
| 2 | chr1: 215854466-215861792 | USH2A | 27,988-28,001 |
| 3 | chr2: 51266798-51339236 | upstream of NRXN1 | 32,494-32,587 |
| 4 | chr3: 172591359-172604675 | downstream of SPATA16 | N/A |
| 5 | chr4: 189084240-189117031 | downstream of TRIML1 | N/A |
| 6 | chr6: 7461346-7470321 | between RIOK1 and DSP | 62,966-62,998 |
| 7 | chr6: 62426827-62472074 | KHDRBS2 | N/A |
| 8 | chr6: 147577803-147684318 | STXBP5 | N/A |
| 9 | chr7: 6870635-6871412 | upstream of CCZ1B | 69,319-69,561 |
| 10 | chr7: 93070811-93116320 | CALCR, MIR653, MIR489 | N/A |
| 11 | chr9: 28207468-28348133 | LINGO2 | N/A |
| 12 | chr9: 28354180-28354967 | LINGO2 (intron) | N/A |
| 13 | chr10: 83886963-83888343 | NRG3 (intron) | N/A |
| 14 | chr10: 92262627-92298079 | downstream of BC037970 | N/A |
| 15 | chr12: 102095178-102108946 | CHPT1 | 7410-7426 |
| 16 | chr13: 40089105-40090197 | LHFP (intron) | N/A |
| 17 | chr14: 100705631-100828134 | SLC25A29, YY1, MIR345, | N/A |
| | | SLC25A47, WARS | |
| 18 | chr14: 102018946-102026138 | DIO3AS, DIO3OS | N/A |
| 19 | chr14: 102729881-102749930 | MOK | N/A |
| 20 | chr14: 102973910-102975572 | ANKRD9 (RAGE) | N/A |
| 21 | chr15: 25690465-28513763 | ATP10A, GABRB3, | N/A |
| | | GABRA5, GABRG3, | |
| 22 | chr15: 31092983-31369123 | FAN1, MTMR10, MIR211, | N/A |
| | | TRPM1 | |
| 23 | chr15: 31776648-31822910 | OTUD7A | N/A |
| 24 | chr20: 32210931-32441302 | NECAB3, CBFA2T2, | N/A |
| | | C20orf144, NECAB3, | |

¹Custom probes were only included on the array for some CNVs.
N/A denotes that no custom probes were used.

TABLE 13
Summary of Custom Probes for CNVs from Table 4

| | | | Custom Probe |
|---|---|---|---|
| No. | Region of Highest Significance | Gene/Region | SEQ ID NOs¹ |
| 1 | chr1: 146656292-146707824 | FMO5 | N/A |
| 2 | chr2: 13203874-13209245 | upstream of LOC100506474 | 31,283-31,314 |
| 3 | chr2: 45489954-45492582 | between UNQ6975 and | N/A |
| | | SRBD1 | |
| 4 | chr2: 51237767-51245359 | NRXN1** | N/A |
| 5 | chr2: 62230970-62367720 | COMMD1 | 33,402-39,860 |
| 6 | chr2: 115133493-115140263 | between LOC440900 and | N/A |
| | | DPP10** | |
| 7 | chr3: 1937796-1941004 | between CNTN6 and | N/A |
| | | CNTN4** | |
| 8 | chr3: 67657429-68962928 | SUCLG2, FAM19A4, | N/A |
| | | FAM19A1 | |
| 9 | chr4: 73766964-73816870 | COX18, ANKRD17 | 51,803-52,100 |
| 10 | chr4: 171366005-171471530 | between AADAT** and | N/A |
| | | HSP90AA6P | |
| 11 | chr5: 118527524-118589485 | DMXL1, TNFAIP8 | 61,165-61,290 |
| 12 | chr6: 39069291-39072241 | SAYSD1 | 64,149-64,167 |
| 13 | chr8: 54855680-54912001 | RGS20, TCEA1 | N/A |
| 14 | chr10: 49370090-49471091 | FRMPD2P1, FRMPD2 | N/A |
| 15 | chr10: 50884949-50943185 | OGDHL, C10orf53 | N/A |
| 16 | chr12: 53177144-53180552 | between KRT76 and KRT3 | N/A |
| 17 | chr15: 20192970-20197164 | downstream of HERC2P3 | 12,508-12,563 |
| 18 | chr15: 25099351-25102073 | SNRPN** | N/A |
| 19 | chr15: 25099351-25102073 | SNRPN** | N/A |
| 20 | chr15: 25579767-25581658 | between SNORD109A and | N/A |
| | | UBE3A** | |
| 21 | chr15: 25582882-25662988 | UBE3A** | N/A |
| 22 | chr16: 21958486-22172866 | C16orf52, UQCRC2**, | N/A |
| | | PDZD9, VWA3A | |
| 23 | chr16: 29664753-30177298 | DOC2A**, ASPHD1, | N/A |
| | | LOC440356, TBX6, | |
| | | LOC100271831, PRRT2 | |
| | | CDIPT, QPRT, YPEL3, | |
| | | PPP4C, MAPK3**, SPN, | |
| | | MVP, FAM57B, ZG16, | |
| | | ALDOA, INO80E, SEZ6L2, | |
| | | TAOK2, KCTD13, MAZ, | |
| | | KIF22, GDPD3, C16orf92, | |
| | | C16orf53, TMEM219, | |
| | | C16orf54, HIRIP3 | |
| 24 | chr16: 82423855-82445055 | between MPHOSPH6 and | N/A |
| | | CDH13 | |
| 25 | chr17: 14132271-14133349 | between COX10 and | N/A |
| | | CDRT15 | |
| 26 | chr17: 14132271-15282708 | PMP22**, CDRT15, TEKT3, | N/A |
| | | MGC12916, CDRT7, | |
| | | HS3ST3B1 | |
| 27 | chr17: 14952999-15053648 | between CDRT7 and PMP22 | N/A |
| 28 | chr17: 15283960-15287134 | between TEKT3 and | N/A |
| | | FAM18B2-CDRT4 | |
| 29 | chr20: 8162278-8313229 | PLCB1** | N/A |
| 30 | chrX: 29944502-29987870 | IL1RAPL1** | N/A |
| 31 | chrX: 140329633-140348506 | SPANXC | N/A |
| 32 | chrX: 148882559-148886166 | MAGEA8 | N/A |

¹Custom probes were only included on the array for some CNVs.
N/A denotes that no custom probes were used.

### Example 3

### Use of CNV Data to Select Patients for Treatment with Mitochondrial Therapies

In this study, collective CNV data were used to assess a patient population having diagnoses for autism and/or developmental delay. The population was stratified into groups most likely to respond well to pharmacotherapies in development for mitochondrial disease patients or currently available mitochondrial therapies. The collective CNV data was obtained using the custom clinical array as described in Example 2.

At the time of the study, there were 77 mitochondrial disease-associated nuclear-encoded genes, and 1805 human nuclear mitochondrial genes listed in the NIH Pubmed database with the tag “Mitochondria.”

The patient population consisted of 1,740 patients undergoing clinical evaluation of autism spectrum disorders and/or other disorders of childhood development. Of the 1,740 patients tested, 1,176 patients were evaluated using the Affymetrix Cytoscan HD array or the Affymetrix Cytogenetics 2.7 M array, and 564 were tested using a custom clinical array generated as described above in Example 2. The diagnostic yield of the custom clinical array of clinically reportable copy number variants (CNVs) was 28.9%. Diagnostic yield is the percentage of patients with a clinically relevant CNV divided by the total number of patients tested.

The custom clinical array used herein had the highest probe density of all marketed CMA platforms, and contains probes that provide high enough resolution to detect CNVs affecting a single gene in 45 of the 77 mitochondrial disease-associated nuclear-encoded genes known at the time of the study. It is the only CMA platform with sufficient probe density to detect 4 of these 45 genes.

Size of deletion in CNVs was determined in the following manner. All probes on the custom microarray represent a known chromosomal coordinate based on hg19. See the sequence listing and Table 14 from U.S. Provisional Application 61/977,462 and Table 14 from International PCT Publication No. 2014/055915, the disclosure of each of which is incorporated by reference in their entireties. In an individual who has no deletion or duplication in a particular region, all probes will have a uniform signal that represents having 2 copies of each chromosome at that position. A CNV is detected by looking for increases (duplication) or decreases (deletion) in signal intensity at individual probes, each of which represent a unique location in the genome. When 25 or more probes targeting contiguous regions of the genome show a reduced signal compared to an individual with no CNV, the test individual can then be said to have a deletion at the location containing the probes that have a reduced signal. Since the genomic coordinates of each probe are known, CNV size is determined by the coordinates of the probes showing reduced signal intensity, and the maximal CNV boundaries are defined by the probes nearest to those showing reduced signal that themselves do not show a reduced signal.

In this study, 27 patients, or 1.5% of the patient population, had clinically relevant CNVs that affect mitochondrial disease-associated genes. Furthermore, 185 patients, or 11% of the patient population, had a CNV affecting one or more of the 1805 nuclear genes encoding proteins associated with mitochondrial functions. These patients were further sorted into groups based on the mitochondrial function carried out by genes within their CNVs (Table 15). In Table 15, the chromosome number of the deletion or duplication for each patient is shown, followed by the list of nuclear mitochondrial genes affected by the CNV. One third of these 185 patients had changes in genes involved with electron transport functions or other functions related to regulating oxidative stress. These patients comprise the group most likely to respond to EPI-743 as well as other therapies aimed at relieving oxidative stress.

TABLE 15
Patients identified with changes in mitochondrial genes

| | Chromosome | | | | | | | | | | | |
|---|---|---|---|---|---|---|---|---|---|---|---|---|
| Patient | location of | DEL or | | | | | | | | | | |
| Number | CNV | DUP | Affected Mitochondrial Genes (*mitochondrial disease-associated genes in bold) | | | | | | | | | |
| 1 | chr1 | DUP | DAP3 | LMNA | SEMA4A | SLC25A44 | MEF2D | MRPL24 | NTRK1 | MRPS21P2 | CCDC19 | KCNJ10 |
| | (Patient 1, continued) | | CASQ1 | PEA15 | PPOX | NDUFS2 | TOMM40L | SDHC | | | | |
| 2 | chr13 | DEL | DNAJC15 | ENOX1 | TPT1 | SLC25A30 | TIMM9P3 | SUCLA2 | RB1 | ATP5F1P1 | MRPS31P5 | THSD1P1 |
| | (Patient 2, continued) | | MRPS31P4 | SLC25A5P4 | | | | | | | | |
| 3 | chr15 | DUP | EIF2AK4 | BMF | IVD | MRPL42P5 | RAD51 | RMDN3 | C15orf62 | NDUFAF1 | PLA2G4B | ATP5HP1 |
| | (Patient 3, continued) | | CKMT1B | STRC | CKMT1A | | | | | | | |
| 4 | chr16 | DUP | TUFM | ATP2A1 | SPNS1 | | | | | | | |
| 5 | chr17 | DUP | AIPL1 | ALOX12 | ACADVL | SLC2A4 | PLSCR3 | TMEM102 | | | | |
| 6 | chr17 | DUP | ALOX12 | ACADVL | SLC2A4 | PLSCR3 | TMEM102 | TP53 | WRAP53 | | | |
| 7 | chr17 | DUP | COX10 | | | | | | | | | |
| 8 | chr17 | DUP | COX10 | | | | | | | | | |
| 9 | chr17 | DUP | TTC19 | PLD6 | FLCN | NT5M | PEMT | ATPAF2 | MYPO15A | MIEF2 | SHMT1 | ALDH3A2 |
| | (Patient 9, continued) | | AKAP10 | TMEM11 | MAP2K3 | MTRNR2L1 | | | | | | |
| 10 | chr18 | DUP | TYMS | ENOSF1 | SLC25A3P3 | NDUFV2 | RALBP1 | CIDEA | AFG3L2 | | | |
| 11 | chr2 | DUP | RNASEH1 | CMPK2 | RSAD2 | YWHAQ | DDX1 | HADHA | HADHB | OTOF | SLC35F6 | MPV17 |
| | (Patient 11, continued) | | ZNF513 | MRPL33 | BRE | TRMT61B | C2orf71 | NLRC4 | | | | |
| 12 | chr2 | DEL | IDH1 | ACADL | CPS1 | ERBB4 | | | | | | |
| 13 | chr20 | DUP | MTRNR2L3 | PCK1 | VAPB | TUBB1 | ATP5E | SLMO2-ATP5E | MRPS16P2 | MTG2 | MIR1-1 | PRPF6 |
| 14 | chr22 | DEL | PPARA | TRMU | GRAMD4 | MAPK12 | MAPK11 | SCO2 | TYMP | CPT1B | | |
| 15 | chr22 | DEL | MAPK12 | MAPK11 | SCO2 | TYMP | CPT1B | | | | | |
| 16 | chr22 | DEL | MAPK12 | MAPK11 | SCO2 | TYMP | CPT1B | | | | | |
| 17 | chr3 | DEL | SUCLG2 | | | | | | | | | |
| 18 | chr3 | DEL | MRPL3 | ACAD11 | TF | PCCB | LOC100289118 | | | | | |
| 19 | chrX | DUP | HCCS | LOC100422628 | MRPL35P4 | ATXN3L | CA5B | PDHA1 | SMPX | ACOT9 | PDK3 | GK |
| | (patient 19, continued) | | CYBB | RPGR | OTC | MPC1L | DDX3X | ATP5G2P4 | MAOA | MAOB | FUNDC1 | DUSP21 |
| | | | LOC392452 | RP2 | NDUFB11 | LOC101060049 | MRPL32P1 | HDAC6 | TIMM17B | PQBP1 | PIM2 | LOC101060199 |
| | | | HSD17B10 | LOC100128454 | LOC100288560 | APEX2 | ALAS2 | MTRNR2L10 | LOC644924 | GRPEL2P2 | LOC100128171 | OPHN1 |
| | | | PIN4 | LOC100129272 | ABCB7 | COX7B | ATP7A | POU3F4 | APOOL | MRPS22P1 | PABPC5 | TSPAN6 |
| | | | NOX1 | TIMM8A | ARMCX3 | LOC100420247 | SLC25A53 | PRPS1 | PSMD10 | ACSL4 | AGTR2 | MRPS17P9 |
| | | | SLC25A43 | SLC25A5 | NDUFA1 | GLUD2 | MRRFP1 | XIAP | APLN | AIFM1 | SLC25A14 | TIMM8BP2 |
| | | | LOC100422685 | FATE1 | BCAP31 | ABCD1 | IDH3G | MECP2 | TAZ | TMLHE | | |
| 20 | chrX | DUP | HCCS | LOC100422628 | MRPL35P4 | ATXN3L | CA5B | PDHA1 | SMPX | ACOT9 | PDK3 | GK |
| | (Patient 20, continued) | | CYBB | RPGR | OTC | MPC1L | DDX3X | ATP5G2P4 | MAOA | MAOB | FUNDC1 | DUSP21 |
| | | | LOC392452 | RP2 | NDUFB11 | LOC101060049 | MRPL32P1 | HDAC6 | TIMM17B | PQBP1 | PIM2 | LOC101060199 |
| | | | HSD17B10 | LOC100128454 | LOC100288560 | APEX2 | ALAS2 | MTRNR2L10 | LOC644924 | | | |
| 21 | chrX | DEL | OTC | | | | | | | | | |
| 22 | chrX | DUP | TAZ | | | | | | | | | |
| 23 | chr2 | DUP | PTCD3 | IMMT | MRPL35 | REEP1 | | | | | | |
| 24 | chr6 | DUP | MUT | | | | | | | | | |
| 25 | chr5 | DEL | MCCC2 | | | | | | | | | |
| 26 | chr9 | DEL | GLDC | | | | | | | | | |
| 27 | chr9 | DUP | GLDC | | | | | | | | | |

Genes involved in redox reactions in mitochondria, but not (yet) associated with disease
NDUF* (NADH dehydrogenase ubiquinone)

| 28 | chr16 | DUP | MRPS34 | HAGH | FAHD1 | NDUFB10 | GFER | E4F1 | ECI1 | | | |
|---|---|---|---|---|---|---|---|---|---|---|---|---|
| 29 | chr16 | DUP | MRPS34 | HAGH | FAHD1 | NDUFB10 | GFER | E4F1 | ECI1 | | | |
| 30 | chr19 | DUP | NDUFA3 | PRPF31 | | | | | | | | |
| 31 | chr21 | DUP | NRIP1 | MRPL39 | ATP5J | GABPA | APP | SOD1 | ITSN1 | ATP5O | MRPS6 | RUNX1 |
| | (Patient 31, continued) | | ATP5J2LP | MRPL20P1 | TIMM9P2 | NDUFV3 | MRPL51P2 | C21orf33 | C21orf2 | IMMTP1 | SLC19A1 | S100B |
| 32 | chr22 | DUP | SLC25A5P1 | SMDT1 | NDUFA6 | CYP2D6 | CYB5R3 | ATP5L2 | BIK | MCAT | TSPO | |
| 33 | chr7 | DEL | NDUFA4 | | | | | | | | | |

ATP5* (F1 Complex)

| | | | | | | | | | |
|---|---|---|---|---|---|---|---|---|---|
| 34 | chr14 | DUP | INF2 | SIVA1 | AKT1 | ATP5G1P1 | | | |
| 35 | chr16 | DEL | ATP5A1P3 | DHODH | DHX38 | | | | |
| 36 | chr17 | DUP | ATP5LP6 | | | | | | |
| 37 | chr21 | DEL | ATP5J2LP | MRPL20P1 | | | | | |
| 38 | chr3 | DUP | ATP5G1P3 | | | | | | |
| 39 | chr3 | DEL | TNFSF10 | ATP5G1P4 | | | | | |
| 40 | chr4 | DEL | WFS1 | GRPEL1 | HTRA3 | PROM1 | PPARGC1A | ATP5LP3 | SOD3 |
| 41 | chrY | DUP | TOMM22P2 | ATP5JP1 | MRP63P10 | DDX3Y | TOMM22P1 | SLC25A15P1 | |
| 42 | chrY | DUP | TOMM22P2 | ATP5JP1 | MRP63P10 | DDX3Y | TOMM22P1 | SLC25A15P1 | |
| 43 | chrY | DUP | TOMM22P2 | ATP5JP1 | MRP63P10 | DDX3Y | TOMM22P1 | SLC25A15P1 | |
| 44 | chrY | DUP | TOMM22P2 | ATP5JP1 | MRP63P10 | DDX3Y | TOMM22P1 | SLC25A15P1 | |

Cytochrome c reductase

| 45 | chr1 | DEL | AKT3 | COX20 | | | | | | | | |
|---|---|---|---|---|---|---|---|---|---|---|---|---|
| 46 | chr11 | DUP | SIRT3 | COX8BP | MRPS24P1 | RNH1 | HRAS | MIR210 | TALDO1 | SLC25A22 | CTSD | MRPL23 |
| | (Patient 46, continued) | | IGF2 | INS | CDKN1C | PHLDA2 | STIM1 | | | | | |
| 47 | chr19 | DUP | RDH13 | TNNI3 | COX6B2 | | | | | | | |
| 48 | chr17 | DUP | COA3 | BECN1 | VAT1 | DHX8 | NAGS | SLC25A39 | GFAP | NMT1 | MAPT | |
| 49 | chr16 | DEL | UQCRC2 | | | | | | | | | |
| 50 | chr16 | DEL | UQCRC2 | | | | | | | | | |
| 51 | chr8 | DEL | CYP11B1 | CYP11B2 | TOP1MT | CYC1 | | | | | | |

Mitochondrial solute/metabolite carriers

| | | | | | | | | |
|---|---|---|---|---|---|---|---|---|
| 52 | chr17 | DUP | SLC2A4 | PLSCR3 | TMEM102 | TP53 | WRAP53 | |
| 53 | chr2 | DUP | SLC3A1 | | | | | |
| 54 | chr2 | DUP | SLC25A12 | | | | | |
| 55 | chr22 | DEL | PRODH | SLC25A1 | MRPL40 | C22orf29 | TXNRD2 | AIFM3 |
| 56 | chr22 | DEL | PRODH | SLC25A1 | MRPL40 | C22orf29 | TXNRD2 | AIFM3 |
| 57 | chr22 | DUP | PRODH | SLC25A1 | MRPL40 | C22orf29 | TXNRD2 | AIFM3 |
| 58 | chr22 | DUP | PRODH | SLC25A1 | MRPL40 | C22orf29 | TXNRD2 | AIFM3 |
| 59 | chr22 | DUP | PRODH | SLC25A1 | MRPL40 | C22orf29 | TXNRD2 | AIFM3 |
| 60 | chr22 | DEL | PRODH | SLC25A1 | MRPL40 | C22orf29 | TXNRD2 | AIFM3 |
| 61 | chr22 | DEL | PRODH | SLC25A1 | MRPL40 | C22orf29 | TXNRD2 | AIFM3 |
| 62 | chr22 | DEL | PRODH | SLC25A1 | MRPL40 | C22orf29 | TXNRD2 | AIFM3 |
| 63 | chr22 | DEL | PRODH | SLC25A1 | MRPL40 | C22orf29 | TXNRD2 | AIFM3 |
| 64 | chr22 | DEL | SLC25A1 | MRPL40 | C22orf29 | TXNRD2 | AIFM3 | |
| 65 | chr22 | DEL | SLC25A1 | MRPL40 | C22orf29 | TXNRD2 | AIFM3 | |
| 66 | chr17 | DUP | TIMM22 | | | | | |
| 67 | chr3 | DUP | SLC25A26 | | | | | |
| 68 | chrX | DEL | MRPS17P9 | SLC25A43 | | | | |

Mitochondrial ATPases/Energy Metabolism

| | | | | | | | | |
|---|---|---|---|---|---|---|---|---|
| 69 | chr1 | DEL | AURKAIP1 | MRPL20 | ATAD3C | ATAD3B | ATAD3A | PRKCZ |
| 70 | chr9 | DUP | LOC138234 | AK3 | GLDC | LOC138864 | | |
| 71 | chr9 | DEL | LOC138234 | AK3 | GLDC | | | |

Thioredoxin

| | | | | |
|---|---|---|---|---|
| 72 | chr1 | DUP | TXNIP | PDZK1 |
| 73 | chr1 | DEL | TXNIP | PDZK1 |

Ribosomal Complex Proteins

| | | | | | | |
|---|---|---|---|---|---|---|
| 74 | chr10 | DEL | BNIP3 | ECHS1 | MTG1 | CYP2E1 |
| 75 | chr16 | DEL | MPG | HBA2 | PDIA2 | MRPL28 |
| 76 | chr17 | DUP | MYO19 | MRM1 | | |
| 77 | chr17 | DUP | MYO19 | MRM1 | | |
| 78 | chr2 | DUP | TIMM8AP1 | IFIH1 | | |
| 79 | chr6 | DEL | MRPS18B | DHX16 | | |
| 80 | chr7 | DEL | MRPS17 | | | |

Creatine Kinase

| | | | | |
|---|---|---|---|---|
| 81 | chr15 | DEL | CKMT1B | STRC |
| 82 | chr15 | DEL | CKMT1B | STRC |

Apoptosis related

| | | | | | |
|---|---|---|---|---|---|
| 83 | chr12 | DEL | GABARAPL1 | BCL2L14 | DDX47 |
| 84 | chr15 | DUP | DUT | | |
| 85 | chr10 | DUP | VDAC2 | | |
| 86 | chr16 | DUP | WWOX | | |
| 87 | chr16 | DEL | WWOX | | |
| 88 | chr16 | DEL | WWOX | | |
| 89 | chr17 | DUP | YWHAE | | |
| 90 | chr2 | DEL | BCL2L11 | MERTK | |
| 91 | chr2 | DUP | BCL2L11 | MERTK | |
| 92 | chr22 | DEL | CHEK2 | HSCB | |
| 93 | chr3 | DUP | FHIT | | |
| 94 | chr3 | DUP | FHIT | | |
| 95 | chr3 | DUP | FHIT | LOC101060206 | |
| 96 | chr3 | DEL | FHIT | | |
| 97 | chr9 | DUP | NAIF1 | SLC25A25 | |
| 98 | chr2 | DUP | PRKCE | | |

Glutathione S transferase family

| | | | | |
|---|---|---|---|---|
| 99 | chr12 | DEL | MGST1 | LOC390298 |

Maturation of OXPHOS proteins

| | | | |
|---|---|---|---|
| 100 | chr13 | DEL | MIPEP |

Protection from Oxidative Stress

| | | | | | | | | |
|---|---|---|---|---|---|---|---|---|
| 101 | chr16 | DUP | MPV17L | NDE1 | | | | |
| 102 | chr16 | DUP | MPV17L | NDE1 | | | | |
| 103 | chr16 | DUP | MPV17L | NDE1 | | | | |
| 104 | chr16 | DUP | MPV17L | NDE1 | | | | |
| 105 | chr16 | DUP | MPV17L | NDE1 | | | | |
| 106 | chr16 | DUP | MPV17L | NDE1 | | | | |
| 107 | chr16 | DEL | MPV17L | NDE1 | | | | |
| 108 | chr16 | DUP | MPV17L | NDE1 | | | | |
| 109 | chr16 | DUP | MPV17L | NDE1 | | | | |
| 110 | chr16 | DUP | MPV17L | NDE1 | | | | |
| 111 | chr16 | DUP | MPV17L | NDE1 | | | | |
| 112 | chr16 | DEL | CA5A | | | | | |
| 113 | chr22 | DEL | PRODH | SLC25A1 | MRPL40 | C22orf29 | TXNRD2 | AIFM3 |
| 114 | chr22 | DEL | PRODH | SLC25A1 | MRPL40 | C22orf29 | TXNRD2 | AIFM3 |
| 115 | chr22 | DUP | PRODH | SLC25A1 | MRPL40 | C22orf29 | TXNRD2 | AIFM3 |
| 116 | chr22 | DUP | PRODH | SLC25A1 | MRPL40 | C22orf29 | TXNRD2 | AIFM3 |
| 117 | chr22 | DUP | PRODH | SLC25A1 | MRPL40 | C22orf29 | TXNRD2 | AIFM3 |
| 118 | chr22 | DEL | PRODH | SLC25A1 | MRPL40 | C22orf29 | TXNRD2 | AIFM3 |
| 119 | chr22 | DEL | PRODH | SLC25A1 | MRPL40 | C22orf29 | TXNRD2 | AIFM3 |
| 120 | chr22 | DEL | PRODH | SLC25A1 | MRPL40 | C22orf29 | TXNRD2 | AIFM3 |
| 121 | chr22 | DEL | PRODH | SLC25A1 | MRPL40 | C22orf29 | TXNRD2 | AIFM3 |
| 122 | chr22 | DEL | SLC25A1 | MRPL40 | C22orf29 | TXNRD2 | AIFM3 | |
| 123 | chr22 | DEL | SLC25A1 | MRPL40 | C22orf29 | TXNRD2 | AIFM3 | |
| 124 | chr2 | DEL | OLA1 | | | | | |
| 125 | chr4 | DEL | SPATA18 | NOA1 | POLR2B | | | |
| 126 | chr8 | DEL | IL7 | MRPS28 | DECR1 | CALB1 | | |
| 127 | chr16 | DUP | MAPK3 (?) | | | | | |
| 128 | chr16 | DEL | MAPK3 (?) | | | | | |
| 129 | chr16 | DEL | MAPK3 (?) | | | | | |
| 130 | chr16 | DEL | MAPK3 (?) | | | | | |
| 131 | chr16 | DUP | MAPK3 (?) | | | | | |
| 132 | chr16 | DEL | MAPK3 (?) | | | | | |
| 133 | chr16 | DUP | MAPK3 (?) | | | | | |
| 134 | chr16 | DEL | MAPK3 (?) | | | | | |
| 135 | chr16 | DEL | CREBBP (?) | | | | | |
| 136 | chr22 | DEL | MAPK1 (?) | | | | | |
| 137 | chr22 | DEL | MAPK1 (?) | | | | | |

Mitochondrial Fatty Acid Synthesis

| | | | | | | |
|---|---|---|---|---|---|---|
| 138 | chr16 | DUP | ACSF3 | SPG7 | TUBB3 | |
| 139 | chr2 | DUP | GPAT2 | STARD7 | TMEM127 | SNRNP200 |

Mitochondrial nucleotidase

| | | | | | |
|---|---|---|---|---|---|
| 140 | chr17 | DEL | PLD6 | FLCN | NT5M |
| 141 | chr2 | DUP | RNASEH1 | | |

ABC (ATP Binding Cassette) Transporters

| | | | | | |
|---|---|---|---|---|---|
| 142 | chr17 | DEL | ABCA8 | | |
| 143 | chr2 | DUP | ABCA12 | | |
| 144 | chr7 | DUP | TMEM243 | ABCB4 | ABCB1 |

Heme biosynthesis

| | | | |
|---|---|---|---|
| 145 | chr3 | DUP | CPOX |

Humanin Family of Mitochondrial Peptides

| | | | | |
|---|---|---|---|---|
| 146 | chr5 | DUP | MTX3 | MTRNR2L2 |

Mitochondrial maintenance

| | | | | |
|---|---|---|---|---|
| 147 | chr6 | DUP | PARK2 | |
| 148 | chr7 | DUP | MAD1L1 | NUDT1 |
| 149 | chr7 | DEL | CHCHD3 | |
| 150 | chr8 | DUP | MICU3 | |

Immune Response

| | | | |
|---|---|---|---|
| 151 | chr7 | DUP | EZH2 |

4p- Cohort

| 153 | chr4 | DEL | PDE6B | ATP5I | LETM1 | NAT8L | HTT | WFS1 | GRPEL1 | HTRA3 | PROM1 | |
|---|---|---|---|---|---|---|---|---|---|---|---|---|
| 154 | chr4 | DEL | PDE6B | ATP5I | LETM1 | NAT8L | HTT | WFS1 | GRPEL1 | HTRA3 | PROM1 | |
| 155 | chr4 | DEL | PDE6B | ATP5I | LETM1 | NAT8L | HTT | WFS1 | GRPEL1 | HTRA3 | | |
| 156 | chr4 | DEL | PDE6B | ATP5I | LETM1 | NAT8L | HTT | WFS1 | GRPEL1 | HTRA3 | PROM1 | |
| 157 | chr4 | DEL | PDE6B | ATP5I | LETM1 | NAT8L | | | | | | |
| 158 | chr4 | DEL | PDE6B | ATP5I | LETM1 | NAT8L | HTT | | | | | |
| 159 | chr4 | DEL | PDE6B | ATP5I | LETM1 | NAT8L | | | | | | |
| 160 | chr4 | DEL | PDE6B | ATP5I | | | | | | | | |
| 161 | chr4 | DEL | PDE6B | ATP5I | LETM1 | NAT8L | | | | | | |
| 162 | chr4 | DEL | PDE6B | ATP5I | LETM1 | NAT8L | HTT | | | | | |
| 163-de- | chr4 | DEL | PDE6B | ATP5I | LETM1 | NAT8L | HTT | WFS1 | GRPEL1 | HTRA3 | PROM1 | PPARGC1A |
| ceased | | | | | | | | | | | | |
| | (Patient 163, continued) | | ATP5LP3 | SOD3 | MRPL51P1 | | | | | | | |
| 164 | chr4 | DEL | PDE6B | ATP5I | LETM1 | NAT8L | HTT | | | | | |
| 165 | chr4 | DEL | PDE6B | ATP5I | LETM1 | NAT8L | HTT | WFS1 | GRPEL1 | HTRA3 | PROM1 | |
| 166 | chr4 | DEL | PDE6B | ATP5I | | | | | | | | |
| 167 | chr4 | DEL | PDE6B | ATP5I | LETM1 | NAT8L | | | | | | |
| 168 | chr4 | DEL | PDE6B | ATP5I | LETM1 | NAT8L | HTT | WFS1 | GRPEL1 | HTRA3 | | |
| 169 | chr4 | DEL | PDE6B | ATP5I | LETM1 | NAT8L | HTT | | | | | |
| 170 | chr4 | DEL | PDE6B | ATP5I | LETM1 | NAT8L | | | | | | |
| 171 | chr4 | DEL | PDE6B | ATP5I | LETM1 | | | | | | | |
| 172 | chr4 | DEL | PDE6B | ATP5I | LETM1 | NAT8L | HTT | WFS1 | GRPEL1 | HTRA3 | | |
| 173 | chr4 | DEL | PDE6B | ATP5I | LETM1 | NAT8L | HTT | WFS1 | GRPEL1 | HTRA3 | | |
| 174 | chr4 | DEL | PDE6B | ATP5I | LETM1 | NAT8L | HTT | WFS1 | | | | |
| 175 | chr4 | DEL | PDE6B | ATP5I | LETM1 | NAT8L | HTT | | | | | |
| 176 | chr4 | DEL | PDE6B | ATP5I | LETM1 | NAT8L | HTT | WFS1 | GRPEL1 | HTRA3 | PROM1 | |
| 177 | chr4 | DEL | PDE6B | ATP5I | LETM1 | NAT8L | HTT | | | | | |
| 178 | chr4 | DEL | PDE6B | ATP5I | LETM1 | NAT8L | HTT | WFS1 | GRPEL1 | HTRA3 | | |
| 179 | chr4 | DEL | PDE6B | ATP5I | LETM1 | NAT8L | HTT | | | | | |
| 180 | chr4 | DEL | PDE6B | ATP5I | LETM1 | NAT8L | HTT | WFS1 | GRPEL1 | | | |
| 181 | chr4 | DEL | PDE6B | ATP5I | LETM1 | NAT8L | HTT | | | | | |
| 182 | chr4 | DEL | LETM1 | | | | | | | | | |
| 183 | chr4 | DEL | LETM1 | NAT8L | HTT | WFS1 | GRPEL1 | HTRA3 | | | | |
| 184 | chr4 | DEL | LETM1 | NAT8L | HTT | | | | | | | |
| 185 | chr4 | DEL | LETM1 | | | | | | | | | |

In this study, a genetically well-defined patient cohort was identified, that would benefit from EPI-743 or other mitochondrial pharmacotherapy (Table 15). This cohort represents 11% of the patient population, a surprising frequency since these patients were not selected for testing based on a suspicion of mitochondrial dysfunction but rather based on generalized clinical symptomology of ASD and/or other disorders of childhood development. The estimated incidence of mitochondrial disease in the general population is about 1 in 10,000. In addition to these patients' genotypes, the available phenotypic data in the form of doctor-reported ICD-9 codes for these patients encompass an array of traits that significantly overlap with phenotypic characteristics of children diagnosed with mitochondrial disease who have already been shown to be excellent responders to EPI-743 (Table 16). These phenotypic characteristics also overlap with the phenotypic traits exhibited by autistic patients and patients with other developmental disorders. This overlap can lead to doctors diagnosing a patient with an ASD rather than with a mitochondrial disease.

TABLE 16
Doctor-reported ICD-9 codes for patients with
CNVs affecting nuclear mitochondrial genes

| Patient | ICD-9 | ICD-9 |
|---|---|---|
| No. | (Primary listed) | Other |
| 1 | 0 | 237.70 - Neurofibromatosis, unspecified |
| 2 | 0 | 279.11 - DiGeorge Syndrome |
| 3 | 0 | 279.11 - DiGeorge Syndrome |
| 4 | 0 | 315.39 - Other developmental speech |
| | | or language disorder |
| 5 | 0 | 315.9 - Unspecified delay in |
| | | development |
| 6 | 0 | 315.9 - Unspecified delay in |
| | | development |
| 7 | 0 | 315.9 - Unspecified delay in |
| | | development |
| 8 | 0 | 315.9 - Unspecified delay in |
| | | development |
| 9 | 0 | 333.99 - Other extrapyramidal diseases |
| | | and abnormal movement disorders |
| 10 | 0 | 348.30 - Encephalopathy, unspecified |
| 11 | 0 | 758.39 - Other autosomal deletions |
| 12 | 0 | 780.39 - Other Convulsions |
| 13 | 0 | 783.42 - Delayed Milestones |
| 14 | 0 | 783.42 - Delayed Milestones |
| 15 | 0 | 783.42 - Delayed Milestones |
| 16 | 0 | 783.42 - Delayed Milestones |
| 17 | 0 | 279.49 - Autoimmune disease, not |
| | | elsewhere classified, 279.9 - |
| | | Unspecified disorder of immune |
| | | mechanism |
| 18 | 0 | 299.01 - Autistic disorder, residual |
| | | state, 345.1 - Generalized convulsive |
| | | epilepsy |
| 19 | 0 | 315.39 - Other developmental speech |
| | | or language disorder, 783.40 - Lack of |
| | | normal physiological development, |
| | | unspecified |
| 20 | 0 | 315.9 - Unspecified delay in |
| | | development, 780.39 - Other |
| | | convulsions |
| 21 | 0 | 315.9 - Unspecified delay in |
| | | development, 780.39 - Other |
| | | convulsions |
| 22 | 0 | 315.9 Unspecified delay in |
| | | development, 783.42 - Delayed |
| | | milestones |
| 23 | 0 | 343.9 - Infantile cerebral palsy, |
| | | unspecified, 758.39 - Other autosomal |
| | | deletions |
| 24 | 0 | 438.10 - Late effects of cerebrovascular |
| | | disease, speech and language deficit, |
| | | unspecified, 438.0 - Late effects of |
| | | cerebrovascular disease, cognitive |
| | | deficits, 728.9 - Unspecified disorder |
| | | of muscle, ligament, and fascia, 300.00 - |
| | | Anxiety state, unspecified, 314.01 - |
| | | Attention deficit disorder with |
| | | hyperactivity |
| 25 | 0 | 745.2 - Tetralogy of fallot, 335.0 - |
| | | Werdnig-Hoffmann disease, 386.19 - |
| | | Other peripheral vertigo |
| 26 | 0 | 749.00 - Cleft palate, unspecified; |
| | | 744.9 - Unspecified congenital |
| | | anomalies of face and neck |
| 27 | 0 | 779.7 - Periventricular leukomalacia, |
| | | 335.0 - Werdnig-Hoffmann disease |
| 28 | 0 | 780.39- Other convulsions, 783.40 - |
| | | Lack of normal physiological |
| | | development, unspecified |
| 29 | 0 | 780.39 - Other convulsions, 758.9 - |
| | | Conditions due to anomaly of |
| | | unspecified chromosome, 279.00 - |
| | | Hypogammaglobulinemia, unspecified |
| 30 | 0 | 783.40 - Lack of normal physiological |
| | | development, unspecified, 728.9 - |
| | | Unspecified disorder of muscle, |
| | | ligament, and fascia |
| 31 | 0 | 783.40 - Lack of normal physiological |
| | | development, unspecified, 783.43 - |
| | | short stature, 749.23 - Cleft palate with |
| | | cleft lip, bilateral, complete |
| 32 | 0 | 783.42 - Delayed milestones, 781.3 - |
| | | Lack of coordination |
| 33 | 0 | 783.42 - Delayed milestones, 783.40 - |
| | | Lack of normal physiological |
| | | development, unspecified |
| 34 | 0 | 783.42 - Delayed milestones, 426.11 - |
| | | First degree atrioventricular block, |
| | | 378.9 - Unspecified disorder of eye |
| | | movements |
| 35 | 0 | 784.69 - Other symbolic dysfunction, |
| | | 744.9 - Unspecified congenital |
| | | anomalies of face and neck, 749.02 - |
| | | Cleft palate, unilateral, incomplete |
| 36 | 0 | 795.2 - Nonspecific abnormal findings |
| | | on chromosomal analysis, 783.1 - |
| | | Abnormal weight gain |
| 37 | 0 | v18.9 - Family history of genetic |
| | | disease carrier |
| 38 | 0 | 786.09 - Other respiratory |
| | | abnormalities, v71.02 - Observation |
| | | for childhood or adolescent antisocial |
| | | behavior, 760.71 - Alcohol affecting |
| | | fetus or newborn via placenta or breast |
| | | milk |
| 39 | 0 | 335.0 - Werdnig-Hoffmann disease |
| 40 | 299.00-Autism, current or active | 0 |
| 41 | 299.00-Autism, current or active | 0 |
| 42 | 299.00-Autism, current or active | 0 |
| 43 | 299.00-Autism, current or active | 0 |
| 44 | 299.00-Autism, current or active | 0 |
| 45 | 299.00-Autism, current or active | 0 |
| 46 | 299.00-Autism, current or active | 0 |
| 47 | 299.00-Autism, current or active | 0 |
| 48 | 299.00-Autism, current or active | 0 |
| 49 | 299.00-Autism, current or active | 0 |
| 50 | 299.00-Autism, current or active | 0 |
| 51 | 299.00-Autism, current or active | 0 |
| 52 | 299.00-Autism, current or active | 0 |
| 53 | 299.00-Autism, current or active | 0 |
| 54 | 299.00-Autism, current or active | 0 |
| 55 | 299.00-Autism, current or active | 0 |
| 56 | 299.00-Autism, current or active | 0 |
| 57 | 299.00-Autism, current or active | 0 |
| 58 | 299.00-Autism, current or active | 0 |
| 59 | 299.00-Autism, current or active | 0 |
| 60 | 299.00-Autism, current or active | 0 |
| 61 | 299.00-Autism, current or active | 0 |
| 62 | 299.00-Autism, current or active | 0 |
| 63 | 299.00-Autism, current or active | 0 |
| 64 | 299.00-Autism, current or active | 0 |
| 65 | 299.00-Autism, current or active | 0 |
| 66 | 299.00-Autism, current or active | 0 |
| 67 | 299.00-Autism, current or active | 299 |
| 68 | 299.00-Autism, current or active | 315.9 |
| 69 | 299.00-Autism, current or active | 315.9 |
| 70 | 299.00-Autism, current or active | 315.9 |
| 71 | 299.00-Autism, current or active | 756 |
| 72 | 299.00-Autism. current or active | 758.32 |
| 73 | 299.00-Autism, current or active | 758.9 |
| 74 | 299.00-Autism, current or active | 783.42 |
| 75 | 299.00-Autism, current or active | 349.82, 768.72, 348.30 |
| 76 | 299.00-Autism, current or active | 780.39, 315.9 |
| 77 | 299.00-Autism, current or active; | 0 |
| | 312.9-Behavior/Conduct disorder | |
| 78 | 299.00-Autism, current or active; | 345 |
| | 312.9-Behavior/Conduct disorder | |
| 79 | 299.00-Autism, current or active; | 0 |
| | 312.9-Behavior/Conduct disorder; | |
| | 319.0-Unspecified mental retardation | |
| 80 | 299.00-Autism, current or active; | 0 |
| | 312.9-Behavior/Conduct disorder; 345- | |
| | Gen. nonconvulsive epilepsy; 742.1- | |
| | Microcephaly | |
| 81 | 299.00-Autism, current or active; | 0 |
| | 312.9-Behavior/Conduct disorder; | |
| | 781.2-Gait abnormality | |
| 82 | 299.00-Autism, current or active; | 0 |
| | 315.5-Mixed developmental disorder | |
| 83 | 299.00-Autism, current or active; | 0 |
| | 315.8-Other specified delays in dev.; | |
| | 783.42-Delayed-Milestones | |
| 84 | 299.00-Autism, current or active; | 0 |
| | 315.9-Unspecified delay in | |
| | development | |
| 85 | 299.00-Autism, current or active; | 781.3 |
| | 315.9-Unspecified delay in | |
| | development | |
| 86 | 299.00-Autism, current or active; | 315.39 |
| | 315.9-Unspecified delay in | |
| | development; 319.0-Unspecified | |
| | mental retardation | |
| 87 | 299.00-Autism, current or active; | 0 |
| | 315.9-Unspecified delay in | |
| | development; 319.0-Unspecified | |
| | mental retardation; 759.7-Multiple | |
| | congenital anomalies | |
| 88 | 299.00-Autism, current or active; | 780.39, 334.3 |
| | 319.0-Unspecified mental retardation | |
| 89 | 299.00-Autism, current or active; | 0 |
| | 319.0-Unspecified mental retardation; | |
| | 345-Gen. nonconvulsive epilepsy | |
| 90 | 299.00-Autism, current or active; 345- | 0 |
| | Gen. nonconvulsive epilepsy | |
| 91 | 299.00-Autism, current or active; 345- | 0 |
| | Gen. nonconvulsive epilepsy | |
| 92 | 299.00-Autism, current or active; | 0 |
| | 759.83-Fragile X syndrome | |
| 93 | 312.9-Behavior/Conduct disorder | 0 |
| 94 | 312.9-Behavior/Conduct disorder | 0 |
| 95 | 312.9-Behavior/Conduct disorder | 0 |
| 96 | 312.9-Behavior/Conduct disorder | 758.81 |
| 97 | 312.9-Behavior/Conduct disorder | 315.9, 756.0, 348.0 |
| 98 | 312.9-Behavior/Conduct disorder; | 783.42 |
| | 314.01-ADHD | |
| 99 | 312.9-Behavior/Conduct disorder; | 0 |
| | 319.0-Unspecified mental retardation | |
| 100 | 312.9-Behavior/Conduct disorder; | 0 |
| | 759.7-Multiple congenital anomalies; | |
| | 783.42-Delayed-Milestones | |
| 101 | 312.9-Behavior/Conduct disorder; | 0 |
| | 781.0-Abnormal involuntary | |
| | movements | |
| 102 | 314.01-ADHD; 315.2-Other specific | 311, 783.40 |
| | learning difficulti | |
| 103 | 314.01-ADHD; 315.9-Unspecified | 0 |
| | delay in development; 759.7-Multiple | |
| | congenital anomalies | |
| 104 | 315.4-Coordination disorder: | 781.3 |
| | Clumsiness; 315.9-Unspecified delay | |
| | in development | |
| 105 | 315.4-Coordination disorder: | 0 |
| | Clumsiness; 728.9-Hypotonia | |
| 106 | 315.8-Other specified delays in dev. | 0 |
| 107 | 315.8-Other specified delays in dev. | 335 |
| 108 | 315.8-Other specified delays in dev. | 335.0, 745.2 |
| 109 | 315.9-Unspecified delay in | 0 |
| | development | |
| 110 | 315.9-Unspecified delay in | 0 |
| | development | |
| 111 | 315.9-Unspecified delay in | 728.85 |
| | development | |
| 112 | 315.9-Unspecified delay in | 744.9-Dysmorphic features |
| | development | |
| 113 | 315.9-Unspecified delay in | 0 |
| | development; 319.0-Unspecified | |
| | mental retardation | |
| 114 | 315.9-Unspecified delay in | 348.3 |
| | development; 345.5-Simple Partial | |
| | Seizures/Epilepsy | |
| 115 | 315.9-Unspecified delay in | 781.3 |
| | development; 742.1-Microcephaly | |
| 116 | 315.9-Unspecified delay in | 0 |
| | development; 759.7-Multiple | |
| | congenital anomalies | |
| 117 | 315.9-Unspecified delay in | 0 |
| | development; 783.41-Failure-to-Thrive | |
| 118 | 315.9-Unspecified delay in | 0 |
| | development; 783.42-Delayed- | |
| | Milestones | |
| 119 | 319.0-Unspecified mental retardation | 0 |
| 120 | 319.0-Unspecified mental retardation | 0 |
| 121 | 319.0-Unspecified mental retardation | 0 |
| 122 | 319.0-Unspecified mental retardation | 0 |
| 123 | 319.0-Unspecified mental retardation | 0 |
| 124 | 319.0-Unspecified mental retardation | 0 |
| 125 | 319.0-Unspecified mental retardation | 0 |
| 126 | 319.0-Unspecified mental retardation | 0 |
| 127 | 319.0-Unspecified mental retardation | 742.3 |
| 128 | 319.0-Unspecified mental retardation | 783.42 |
| 129 | 319.0-Unspecified mental retardation | 348.3, 780.39 |
| 130 | 319.0-Unspecified mental retardation; | 0 |
| | 345.9-Epilepsy, unspecified; 759.7- | |
| | Multiple congenital anomalies | |
| 131 | 319.0-Unspecified mental retardation; | 0 |
| | 345.9-Epilepsy, unspecified; 759.7- | |
| | Multiple congenital anomalies | |
| 132 | 319.0-Unspecified mental retardation; | 0 |
| | 345.9-Epilepsy, unspecified; 759.7- | |
| | Multiple congenital anomalies | |
| 133 | 319.0-Unspecified mental retardation; | 0 |
| | 345.9-Epilepsy, unspecified; 759.7- | |
| | Multiple congenital anomalies | |
| 134 | 319.0-Unspecified mental retardation; | 0 |
| | 345.9-Epilepsy, unspecified; 759.7- | |
| | Multiple congenital anomalies | |
| 135 | 319.0-Unspecified mental retardation; | 0 |
| | 345.9-Epilepsy, unspecified; 759.7- | |
| | Multiple congenital anomalies | |
| 136 | 319.0-Unspecified mental retardation; | 0 |
| | 759.7-Multiple congenital anomalies | |
| 137 | 319.0-Unspecified mental retardation; | 0 |
| | 759.7-Multiple congenital anomalies | |
| 138 | 319.0-Unspecified mental retardation; | 0 |
| | 759.7-Multiple congenital anomalies | |
| 139 | 319.0-Unspecified mental retardation; | 0 |
| | 759.7-Multiple congenital anomalies | |
| 140 | 319.0-Unspecified mental retardation; | 0 |
| | 759.7-Multiple congenital anomalies | |
| 141 | 319.0-Unspecified mental retardation; | 0 |
| | 759.7-Multiple congenital anomalies | |
| 142 | 319.0-Unspecified mental retardation; | 0 |
| | 759.7-Multiple congenital anomalies | |
| 143 | 319.0-Unspecified mental retardation; | 586 |
| | 759.7-Multiple congenital anomalies | |
| 144 | 319.0-Unspecified mental retardation; | 780.39 |
| | 759.7-Multiple congenital anomalies | |
| 145 | 319.0-Unspecified mental retardation; | 780.39 |
| | 759.7-Multiple congenital anomalies | |
| 146 | 319.0-Unspecified mental retardation; | 780.39 |
| | 759.7-Multiple congenital anomalies | |
| 147 | 319.0-Unspecified mental retardation; | 780.39 |
| | 759.7-Multiple congenital anomalies | |
| 148 | 319.0-Unspecified mental retardation; | 780.39 |
| | 759.7-Multiple congenital anomalies | |
| 149 | 319.0-Unspecified mental retardation; | 780.39 |
| | 759.7-Multiple congenital anomalies | |
| 150 | 319.0-Unspecified mental retardation; | 780.39 |
| | 759.7-Multiple congenital anomalies | |
| 151 | 319.0-Unspecified mental retardation; | 780.39 |
| | 759.7-Multiple congenital anomalies | |
| 152 | 345-Gen. nonconvulsive epilepsy | 742.2 |
| 153 | 345-Gen. nonconvulsive epilepsy; | 318.0, 315.34 |
| | 742.1-Microcephaly; 759.7-Multiple | |
| | congenital anomalies | |
| 154 | 345.4-Complex Partial | 0 |
| | Seizures/Epilepsy | |
| 155 | 345.6-Infantile spasms | 0 |
| 156 | 345.9-Epilepsy, unspecified; 759.7- | 315.9 |
| | Multiple congenital anomalies | |
| 157 | 356.1-Charcot-Marie-Tooth disease | 315.9, |
| 158 | 728.9-Hypotonia | 0 |
| 159 | 728.9-Hypotonia | 0 |
| 160 | 728.9-Hypotonia | 315.9 |
| 161 | 728.9-Hypotonia | 783.42 744.9 530.81 |
| 162 | 728.9-Hypotonia | 783.42, 728.5 |
| 163 | 728.9-Hypotonia; 742.1-Microcephaly; | 0 |
| | 781.2-Gait abnormality | |
| 164 | 728.9-Hypotonia; 759.7-Multiple | 0 |
| | congenital anomalies; 781.2-Gait | |
| | abnormality | |
| 165 | 728.9-Hypotonia; 759.81-Prader-Willi | 783.40, |
| | syndrome | |
| 166 | 742.1-Microcephaly | 378.9, 783.42 |
| 167 | 742.1-Microcephaly | 783.42; 787.20; 530.81 |
| 168 | 742.3-Congenital hydrocephalus | 0 |
| 169 | 742.3-Congenital hydrocephalus; | 783.42 |
| | 742.4-Other specified anomalies of | |
| | brain | |
| 170 | 742.4-Other specified anomalies of | 0 |
| | brain | |
| 171 | 742.4-Other specified anomalies of | 783.4 |
| | brain | |
| 172 | 759.7-Multiple congenital anomalies | 315.9 |
| 173 | 759.7-Multiple congenital anomalies | 315.9 |
| 174 | 759.7-Multiple congenital anomalies | 315.9 |
| 175 | 759.7-Multiple congenital anomalies | 315.9 |
| 176 | 759.7-Multiple congenital anomalies | 315.9 |
| 177 | 759.7-Multiple congenital anomalies | 315.9 |
| 178 | 759.7-Multiple congenital anomalies | 758.9 |
| 179 | 759.7-Multiple congenital anomalies | 783.42 |
| 180 | 759.7-Multiple congenital anomalies | 315.9, 358.8 |
| 181 | 759.89-Other specified congenital | F45.22 |
| | anomal | |
| 182 | 783.42-Delayed-Milestones | 0 |
| 183 | 783.42-Delayed-Milestones | 315.31 |
| 184 | 783.42-Delayed-Milestones | 783.40, 752.61 |
| 185 | 784.3-Aphasia | 315.9 |

### Example 4

### Phenotype:Genotype Correlations in Subjects with Syndromic Conditions

CNV data were used to discover new phenotypic correlations associated with specific genotypes, in particular, in patients with syndromic forms of autism and/or developmental delay. These correlations have predictive value in that children with similar CNVs tend to have similar co-morbid conditions as well as similar responses to treatments, thereby allowing caregivers the ability to alter and enhance medical treatment plans based on this new knowledge. Specifically, in this study, children with 4p-Syndrome, also known as Wolf-Hirschhorn Syndrome (WHS), were assessed. However, the methods described here can be generalized to any of the many syndromic microduplication or microdeletion conditions that arise from localized CNVs of variable lengths and phenotypes.

A custom, 2.8M-probe, chromosomal microarray platform (CMA) to finely map CNVs was employed in this study. Probes used in the CMA are provided in the sequence listing and the chromosomal regions to which these probes maps can be found at Table 14 of U.S. Provisional Application 61/977,462 and Table 14 from International PCT Publication No. 2014/055915, the disclosure of each of which is incorporated by reference in their entireties.

Size of deletion in CNVs was determined in the following manner. All probes on the custom microarray represent a known chromosomal coordinate based on hg19. See the sequence listing and Table 14 from U.S. Provisional Application 61/977,462 and Table 14 from International PCT Publication No. 2014/055915, the disclosure of each of which is incorporated by reference in their entireties. In an individual who has no deletion or duplication in a particular region, all probes will have a uniform signal that represents having 2 copies of each chromosome at that position. A CNV deletion is detected by looking for decreases (deletion) in signal intensity at individual probes, each of which represent a unique location in the genome. When 25 or more probes targeting contiguous regions of the genome show a reduced signal compared to an individual with no CNV, the test individual can then be said to have a deletion at the location containing the probes that have a reduced signal. Since the genomic coordinates of each probe are known, CNV size is determined by the coordinates of the probes showing reduced signal intensity, and the maximal CNV boundaries are defined by the probes nearest to those showing reduced signal that themselves do not show a reduced signal.

Wolf-Hirschhorn Syndrome is a rare, multi-genetic disorder that is characterized by a variety of different clinical features. Presentation of the disorder includes: intellectual disability, failure to thrive, seizures, and a characteristic craniofacial facies. The degree to which these “classic” features as well as other co-morbid conditions present themselves in each patient can vary significantly, thereby requiring that the medical management of this disorder be tailored to an individual's needs. Without the benefit of genetic correlation studies of this syndrome, standard medical care for Wolf-Hirschhorn patients means the running of expensive and sometimes invasive medical tests for each patient in order to determine the best course of action. The extent of the chromosomal deletion on the short arm of chromosome 4 is a crucial determining factor for both the severity and the range of phenotypes presented in individuals, but this data is often missed when a diagnosis is made based on the results of a FISH (fluorescence in situ hybridization) test (Ji et al., Chin Med J (Engl) 2010; Maas et al., J. Med Genet. 2008). This FISH test can only indicate the presence or absence of a specific “critical” locus on chromosome 4p, not the size or extent of the deletion. Nor can it detect the presence or absence of any other CNV in the genome. The custom array described herein addresses these needs.

The goal of this study was to examine data from approximately 48 patients with Wolf-Hirschhorn Syndrome and apply novel algorithmic techniques to determine correlations between the patients' finely mapped genetic deletions and their parent-reported phenotypes. This was the largest correlation study to date of phenotypes and treatment outcomes of Wolf-Hirschhorn Syndrome that utilizes genetic data from a customized fine-mapping microarray (as described above in Example 2), at 1 kb resolution.

The patient cohort for this study is provided in the table below.

Patient Cohort for Study Set Forth in Example 5

| | |
|---|---|
| Total Participants | 48 Female:Male (27:21) |
| Average Age: | 11 years (Range: 1-38 years |
| Size of 4p- deletion | 1.3-33.9 Mb |
| Number of genes in deletion | 28-207 |
| Initial diagnosis | Karotype/FISH: 63% (30/48) |
| Patients with second CNV | 29% (14/48) |
| Average size of second CNV | 4.7 Mb |

To score phenotypic data, parent-reported answers to a questionnaire to capture information on >20 different features were used. Correlations between genotypes and phenotypes were observed. Candidate loci were identified using Genome Browser and Ingenuity IPA software. Specifically, patient data was obtained through a partnership with the 4p-Support Group, a nationally run, parent-founded organization, who collected clinical data in the form of a questionnaire called a BioForm, which is completed by member families on a voluntary basis. Data on the Bioform included specific questions about congenital heart disease, renal anomalies that can lead to kidney failure, skeletal dysmorphic features, and other medical conditions that commonly affect this population's medical management and quality of life. The Bioform also collected data concerning parents' experiences with pharmacological and other types of treatments for their child's seizures, which can be severe and life-threatening.

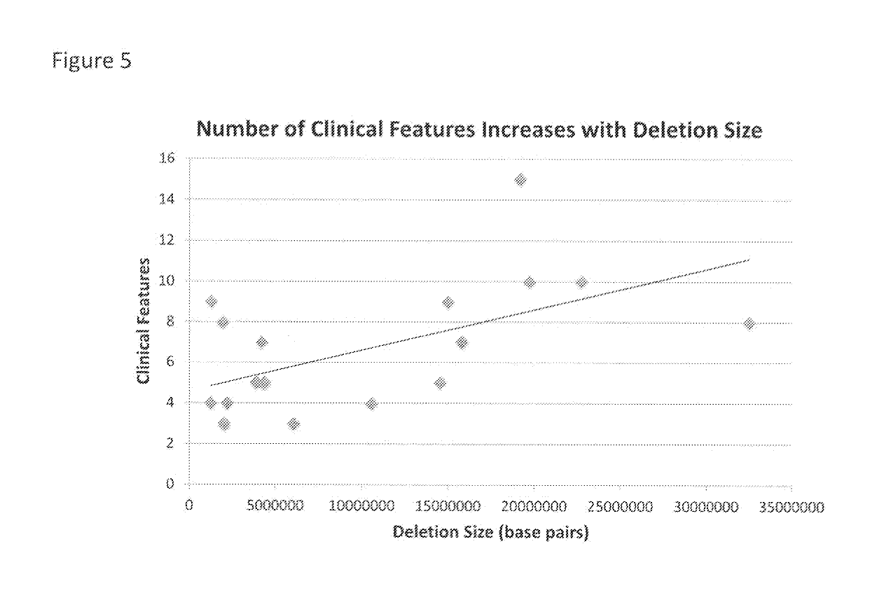

FIG. 5 illustrates the correlation between deletion size and number of clinical features present in the study cohort. The number of patient-family reported clinical features increased with increasing deletion size. Individuals with the 5 smallest deletions had on average 6.2 clinically relevant features compared to individuals with the 5 largest deletions, who had 10.0 clinically relevant features (up to 40% more clinically relevant features based on size of deletion). This correlation suggests that CMA detection, as opposed to FISH technology, has predictive value in the quantity and quality, of clinical manifestations that arise depending on deletion size.

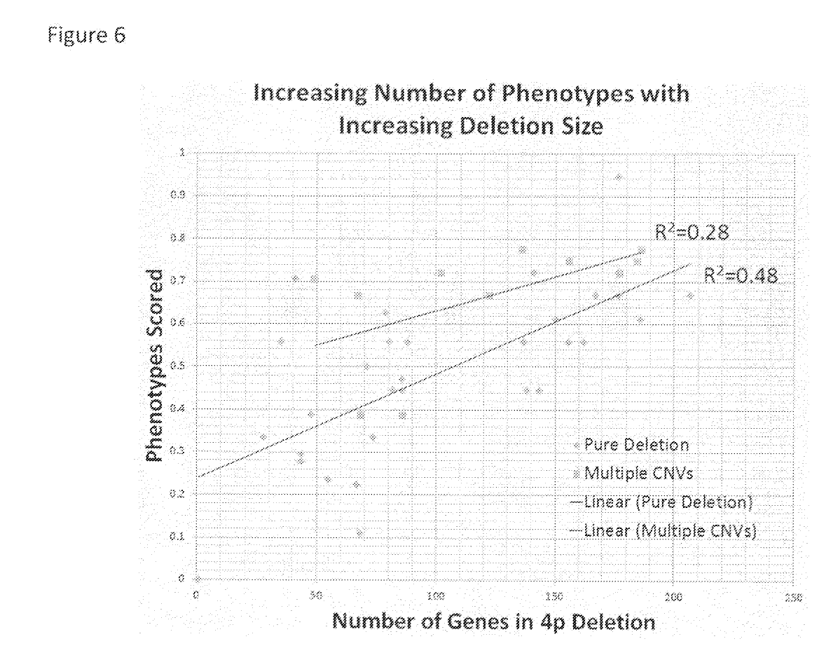

FIG. 6 shows that number of genes in the 4p deletion and the number of phenotypes scored are positively correlated. The deletion size (FIG. 5) and genetic content (FIG. 6) of the deletion uncovered by CMA positively correlates with the number of clinical features of WHS that manifest. This can change medical management of the patient, particularly in terms of symptoms that can be best ameliorated by early detection and treatment (vision loss, seizures, kidney failure).

A second CNV elsewhere in the genome, which co-occurs with a 4p-deletion −30% of the time, increases the number of co-morbid features. Moreover, a second CNV increases the likelihood of having potentially life-threatening status epilepticus (SE) seizures (11/27, or 40%, of individuals with pure deletions report having SE, versus 7/10 individuals with an additional CNV report having SE). Therefore, the CMA can detect second CNVs that co-occur with a 4p deletion. These second CNVs average less than 5 Mb in size, which is below the detection of karyotype and can only be detected by FISH if the second CNV is suspected and specifically probed for. Taken together, this means that by using karyotype/FISH technologies, the second CNV is often missed. Presence of a second CNV correlates with the number of clinical features that manifest, again potentially affecting medical management of the individual. For example, as provided above, the presence of a second CNV increases the chances that the individual may have life-threatening seizures of the status epilepticus type, requiring immediate administration of anti-seizure meds and ER support (to monitor breathing).

Individuals with interstitial deletions not including the terminal 751 kb do not report having seizures (n=4), whereas deletions that encompass the terminus correlate well with seizures (100%).

There are 12 genes in the 751 kb terminal region defined by our work (use of our CMA) that, when lost, correlate with presence of seizures, and when present, correlate with lack of seizures. These candidates lead to the possibility of developing targeted treatments for seizures in these individuals (90% of whom have seizures). Therefore, the position of the CNV in the 4p region, as determined by CMA, is important for medical management and patient prognosis.

One additional individual with a larger interstitial deletion reported having exactly one febrile seizure in 8 years and has been advised by the physician to not take seizure medication since there appears to be little risk. There are 12 genes in this region; of these, bioinformatics analyses indicate PIGG (Phosphotidylinositol glycan anchor biosynthesis, class G) as a candidate seizure-susceptibility gene when deleted along with the WHS critical region(s). Mutations in other members of the GPI anchor biosynthesis pathway cause autosomal recessive disorders (e.g., Mabry Syndrome), all of which have seizures.

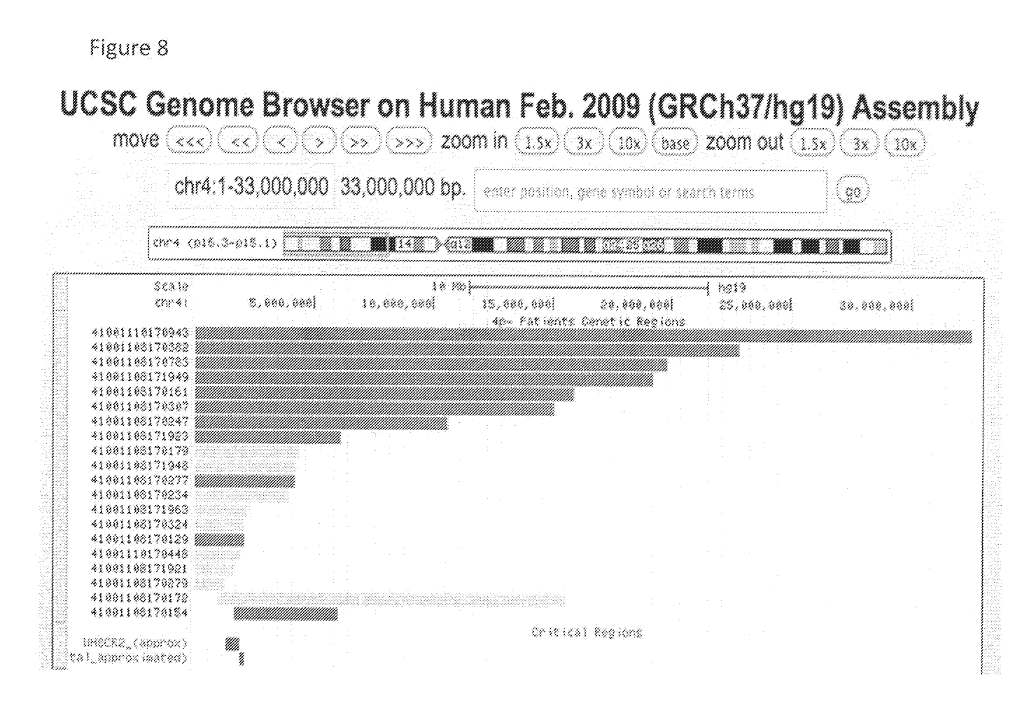

FIG. 8 illustrates the correlation of CMA data with a specific type of clinical manifestation, in this case, congenital heart disease. Each bar on the graph represents the size and location of a patient's 4p-deletion as detected by the customized array provided herein. Black bars indicate patients with congenital heart disease. Gray bars represent patients without congenital heart disease. As shown in FIG. 8, patients with a deletion of 6 MB or larger were more likely to have congenital heart disease than those who had smaller deletions.

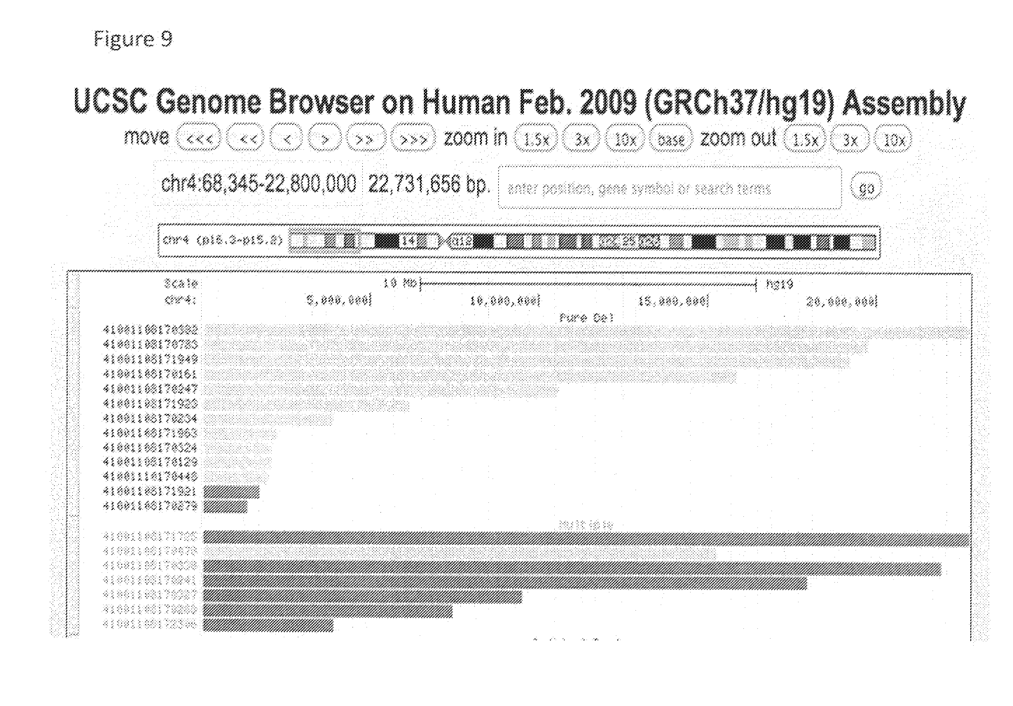

In addition, patients with an additional CNV finding elsewhere in the genome, in addition to the deletion of the 4p terminus, were far more likely to have a debilitating, life-threatening condition known as status epilepticus. Multiple CNV findings occur in about 30% of WHS patients, a significant fraction of the affected population. Patients with status epilepticus are at risk of having prolonged seizures that can lead to death if not taken to an emergency room quickly, within minutes of seizure onset. The knowledge of an increased risk of having a status epilepticus seizure can therefore allow caregivers to prescribe preventative medications as well as respond to seizures quickly. As shown in FIG. 9, patients with multiple CNV findings were more likely to have status epilepticus than patients with only the 4p-deletion. Each horizontal bar on the graph represents the size and location of a patient's 4p-deletion as detected by the customized array provided herein. Black bars indicate patients with status epilepticus. Gray bars represent patients without status epilepticus.

Sophisticated algorithmic tools are used to mine other potential clinical correlations with CNV results. For example, detailed data on over twenty clinical features, including renal disease, intellectual disability, developmental delay, seizures, vision loss and blindness, and other conditions affecting ear, skin, teeth and skeletal development have been collected.

The results of the study have wide-ranging implications for the care of patients affected with Wolf-Hirschhorn syndrome, including better understanding of the genetic causes for certain key features of the syndrome; refining medical practice guidelines for patients based on genetic correlates leading to time-saving and cost-saving measures for both patient families and the insurance industry; defining of best parent-reported treatments for seizures based on patient genotypes; and more broadly, development of powerful software tools and algorithms that can better correlate multiple genes and phenotypes with one another.

### Example 5

### Identification of Best Responders to Mechanistic Drug Therapies

In this study, CNV data were used to identify groups of patients who represent best candidate responders to new mechanism-directed autism drugs in development and on the market. The patient population was stratified into groups that were predicted to respond well to glutamatergic and GABAergic drugs, and those patients that were likely to either not respond or to fare poorly in response to a drug, due to underlying genetics. The approach described in this study has wide-ranging applications to other pharmacotherapies aimed at any genetic disorder detectable by the customized array provided herein, as long as the pharmacotherapy is mechanism-based and the molecular pathways involved are roughly known. In this way, the customized array platform provided herein is a powerful means of delivering personalized medicine: the right drug in the right dose to the right person at the right time, based on genetic knowledge.

Recent developments in the understanding of the etiology of autism indicate that the genetic contribution to this disorder could be as high as 90%. This ‘genetic contribution’ is largely comprised of genes involved in establishing, maintaining and regulating the function of the neural synapse. Furthermore, genetic and electrophysiological studies indicate that autism may arise from an imbalance between excitatory and inhibitory signaling in the brain. In fact, studies using genetic mouse models of autism indicate that key features of autism can arise from either of two scenarios: too much excitatory signaling in the brain, or too little. Drugs are now in development targeted to correct the imbalance. Several drug companies have candidates in various stages of clinical trial development aimed at this mechanism.

Many different genetic changes can lead to the same set of autism-related phenotypes. If imbalance of the excitatory/inhibitory system leads to autism, then one must first determine which side of the imbalance a patient is on, in order for mechanistic drug therapy can be effective and safe. Furthermore, certain forms of autism may arise from mechanisms only peripherally associated with synaptic signaling imbalances, and entirely different pharmacotherapies might be more appropriate for these cases. Decades of studies of drugs that affect glutamatergic signaling in the laboratory indicate that drugs and electrical stimulations that over-excite glutamatergic neurons can lead to hallucinations, seizures and in the worst cases, irreparable neurologic damage and neural cell death. Too little excitatory response, on the other hand, leads to sedation, and a host of other potentially negative side effects.

Table 17 provides predictions for drug responses based on specific genetic changes detectable by the customized array provided herein.

TABLE 17
Predictions for drug response based on genetics

| | Disorders | mGluR5 | mGluR5 | |
|---|---|---|---|---|
| | which can be | antagonist or | agonist or | |
| | clinically | GABA(B) | GABA(B) | |
| | distinguishable | receptor | receptor | |
| Gene | from ASD | agonist | antagonist | Ref |
| FMR1 | Fragile X | Yes | No | Whalley, 2012 |
| | | | | (review) |
| TSC1/2 | Tuberculosis | No | Yes | Auerbach, 2011 |
| Shank3 | Phelan- | No | Yes | Verpelli, 2011 |
| | McDermid | | | |
| | Syndrome | | | |
| SAPAP3 | Autism/DD | Yes | | Wan, 2011 |
| | | (probably) | | |
| Densin180 | Autism/DD | | Yes | Carlisle, 2011 |
| | | | (probably) | |
| GRM5 | ADHD | No | Yes, if | inferred |
| | | | GRM5/+ | |

Table 18 shows the results of querying the 1,400+ patients with CNV results in the database provided herein for CNVs with changes in known glutamatergic/GABAergic signaling genes. 28% of “Abnormal” cases were findings with some relevance to mGluR5/GABA pathway functions. The following were identified: 6 Fragile X patients, 5 Williams-Beuren Syndrome patients, 6 DiGeorge Syndrome patients, 2 Angelman syndrome patients, and 1 each of Rubenstein-Taybi Syndrome, Legius syndrome, Phelan-McDermid Syndrome, CDKL5 deletion, CASK deletion, and EDNRB deletion. These patients, therefore, represent the best candidates for a clinical trial for the use of a glutamate receptor or GABA receptor targeted drug. The effect of the CNV deletion or duplication on excitatory or inhibitory activity of their neurons determines whether an agonist or antagonist is most appropriate.

TABLE 18

| Chromosome | | | | |
|---|---|---|---|---|
| location (gene | Associated condition/ | | | Specific role in GABA, glutamatergic, |
| of Interest) | clinical features | Incidence | Genes | or synapse |
| 7q811.23 | Williams syndrome | Prevalence ~1 in 7,500 | (Many) | Curr Opin Neurol, 2012 April; 25(2); 112-24 |
| | | to 1 in 20,000 births | | |
| 7q11.23 | 7q11.23 duplication | | (Many) | Curr Opin Neurol, 2012 April; 25(2); 112-24 |
| | syndrome, ASD | | | |
| 15q11.2 | Neurodevelopmental | ~1 per 12,000-20,000 | GABRB3, | FMRP/mGluR pathway |
| (UBE3A) | disorder/autism | Angelman syndrome | GABRG3, | |
| | spectrum disorder/ | | GABRA5, | |
| | Angelman syndrome/ | | SNRPN, | |
| | Prader-Willi syndrome | | UBE3A | |
| 15q13.3 | 15q13.3 deletion or | 1 in 100001 in 20000 | CHRNA7 | Loss leads to lower GAD-65 expression in |
| (CHRNA7) | duplication syndrome | | | hippocampus of het. mice. Adams et al, |
| | | | | Neuroscience. 2012 Apr. 5; 207: 274-82. |
| 15q21 | Hirschprung Disease | 1 in 5000 to 1 in 10000 | EDNRB | endothelin receptor type B receives ET-1 |
| (EDNRB) | Type II | (all Hirschprung) | | signal for oxytocin-containing |
| | | | | magnocellular neurons in the SON to |
| | | | | release glutamate J. Neurosci 2010 Dec. 15; |
| | | | | 30(50): 16855-63; they down-regulate |
| | | | | glial glutamate transporters in injured brain |
| | | | | Brain Pathol, 2004 October; 14(4): 406-14 |
| 22q11.2 | DiGeorge syndrome 2 | estimated incidence of | (Many) | Altered dosage of one, or several 22q11 |
| | (Velocardiofacial | one in 4000 births | | mitochondrial genes, particularly during |
| | syndrome 2) | | | early post-natal cortical development, may |
| | | | | disrupt neuronal metabolism or synaptic |
| | | | | signaling Mol Cell Neurosci. 2008; |
| | | | | GABA(B) receptor subunit 1 binds to |
| | | | | proteins affected in 22q11 deletion |
| | | | | syndrome. Zunner D, 2010 March |
| 22q13.31q13.33 | 22q13.3 deletion | There are | SHANK3 | Glut/GABA Synapse stability |
| (SHANK3) | syndrome | approximately 600 | | |
| | (Phelan-McDermid | reported cases of | | |
| | syndrome) | Phelan- McDermid | | |
| | | Syndrome worldwide | | |
| 15q.14 | Legius Syndrome | Unknown, often | SPRED1 | Spred1 is a negative regulator of |
| (SPRED1) | | misdiagnosed as NH | | Ras/Mapk/ERK; required for synaptic |
| | | | | plasticity and hippocampus-dependent |
| | | | | learning. J Neurosci. 2008 Dec. 31; |
| | | | | 28(53): 14443-9 |
| 16p13.3 | Rubinstein-Taybi | Prevalence ~1 per | CREBBP | Downstream effector of mGluR type 1 |
| (CREBBP) | syndrome | 10,000 live births | | receptors in LTP/synaptic plasticity; J |
| | | | | Neurosci. 2012 May |
| Xp11.4 | XLID and FG syndrome | Unknown, several | CASK | In complex with NEGNs/NRXNs |
| (CASK) | | hundred cases | | |
| | | worldwide | | |
| Xq28 | Rett syndrome/MECP2- | ~1 in 10,000 females | MECP2 | FMRP/mGluR pathway |
| (MECP2) | related conditions | (similar, numbers to | | |
| | | ALS, Huntington's, and | | |
| | | Cystic Fibrosis) | | |

The following references are cited and are incorporated by reference in their entireties for all purposes.
1. Rosenberg R E, Law J K, Yenokyan G, McGready J, Kaufmann W E, et al. (2009) Characteristics and Concordance of Autism Spectrum Disorders Among 277 Twin PairsAutism Characteristics and Discordance in Twins. Arch Pediatr Adolesc Med 163: 907-914. doi:10.1001/archpediatrics.2009.98.
2. Hallmayer J, Cleveland S, Torres A, Phillips J, Cohen B, et al. (2011) Genetic Heritability and Shared Environmental Factors Among Twin Pairs With Autism. Arch Gen Psychiatry 68: 1095-1102. doi:10.1001/archgenpsychiatry.2011.76.
3. Lichtenstein P, Carlström E, Rastam M, Gillberg C, Anckarsäter H (2010) The Genetics of Autism Spectrum Disorders and Related Neuropsychiatric Disorders in Childhood. Am J Psychiatry 167: 1357-1363. doi:10.1176/appi.ajp.2010.10020223.
4. Ronald A, Hoekstra R A (2011) Autism spectrum disorders and autistic traits: A decade of new twin studies. Am J Med Genet B Neuropsychiatr Genet 156B: 255-274. doi:10.1002/ajmg.b.31159.
5. International Molecular Genetic Study of Autism Consortium (IMGSAC) (1998) A Full Genome Screen for Autism with Evidence for Linkage to a Region on Chromosome 7q. Hum Mol Genet 7: 571-578. doi:10.1093/hmg/7.3.571.
6. International Molecular Genetic Study of Autism Consortium (IMGSAC) (2001) A Genomewide Screen for Autism: Strong Evidence for Linkage to Chromosomes 2q, 7q, and 16p. Am J Hum Genet 69: 570-581. doi:10.1086/323264.
7. Buxbaum J D, Silverman J, Keddache M, Smith C J, Hollander E, et al. (2003) Linkage analysis for autism in a subset families with obsessive-compulsive behaviors: Evidence for an autism susceptibility gene on chromosome 1 and further support for susceptibility genes on chromosome 6 and 19. Mol Psychiatry 9: 144-150. doi:10.1038/sj.mp.4001465.
8. Martin C L, Ledbetter D H (2007) Autism and cytogenetic abnormalities: solving autism one chromosome at a time. Curr Psychiatry Rep 9: 141-147.
9. Levy D, Ronemus M, Yamrom B, Lee Y, Leotta A. et al. (2011) Rare De Novo and Transmitted Copy-Number Variation in Autistic Spectrum Disorders. Neuron 70: 886-897. doi:10.1016/j.neuron.2011.05.015.
10. Betancur C (2011) Etiological heterogeneity in autism spectrum disorders: More than 100 genetic and genomic disorders and still counting. Brain Res 1380: 42-77. doi:10.1016/j.brainres.2010.11.078.
11. Sanders S J, Murtha M T, Gupta A R, Murdoch J D, Raubeson M J, et al. (2012) De novo mutations revealed by whole-exome sequencing are strongly associated with autism. Nature 485(7397):237-241. doi:10.1038/nature10945
12. Iossifov I, Ronemus M, Levy D, Wang Z, Hakker I, et al. (2012) De Novo Gene Disruptions in Children on the Autistic Spectrum. Neuron 74: 285-299. doi:10.1016/j.neuron.2012.04.009.
13. Girirajan S, Brkanac Z, Coe B P, Baker C, Vives L, et al. (2011) Relative burden of large CNVs on a range of neurodevelopmental phenotypes. PLoS Genet 7: e1002334. doi:10.1371/joumal.pgen.1002334.
14. Sebat J, Lakshmi B, Malhotra D, Troge J, Lese-Martin C, et al. (2007) Strong Association of De Novo Copy Number Mutations with Autism. Science 316: 445-449. doi:10.1126/science.1138659.
15. Marshall C R, Noor A, Vincent J B, Lionel A C, Feuk L, et al. (2008) Structural Variation of Chromosomes in Autism Spectrum Disorder. Am J Hum Genet 82: 477-488. doi:10.1016/j.ajhg.2007.12.009.
16. Christian S L, Brune C W, Sudi J, Kumar R A, Liu S, et al. (2008) Novel Submicroscopic Chromosomal Abnormalities Detected in Autism Spectrum Disorder. Biol Psychiatry 63: 1111-1117. doi:10.1016/j.biopsych.2008.01.009.
17. Glessner J T, Wang K, Cai G, Korvatska O, Kim C E, et al. (2009) Autism genome-wide copy number variation reveals ubiquitin and neuronal genes. Nature 459: 569-573. doi:10.1038/nature07953.
18. Bucan M, Abrahams B S, Wang K, Glessner J T, Herman E I, et al. (2009) Genome-Wide Analyses of Exonic Copy Number Variants in a Family-Based Study Point to Novel Autism Susceptibility Genes. PLoS Genet 5: e1000536. doi:10.1371/journal.pgen.1000536.
19. Pinto D, Pagnamenta A T, Klei L, Anney R, Merico D, et al. (2010) Functional impact of global rare copy number variation in autism spectrum disorders. Nature 466: 368-372. doi:10.1038/nature09146.
20. Szatmari P, Paterson A D, Zwaigenbaum L, Roberts W, Brian J (2007) Mapping autism risk loci using genetic linkage and chromosomal rearrangements. Nat Genet 39: 319-328. doi:10.1038/ng1985.
21. Weiss L A, Shen Y, Korn J M, Arking D E, Miller D T, et al. (2008) Association between Microdeletion and Microduplication at 16p11.2 and Autism. N Engl J Med 358: 667-675. doi:10.1056/NEJMoa075974.
22. Morrow E M, Yoo S-Y, Flavell S W, Kim T-K, Lin Y, et al. (2008) Identifying Autism Loci and Genes by Tracing Recent Shared Ancestry. Science 321: 218-223. doi:10.1126/science.1157657.
23. Jacquemont M-L, Sanlaville D, Redon R, Raoul O, Cormier-Daire V, et al. (2006) Array-based comparative genomic hybridisation identifies high frequency of cryptic chromosomal rearrangements in patients with syndromic autism spectrum disorders. J Med Genet 43: 843-849. doi:10.1136/jmg.2006.043166.
24. Shinawi M, Liu P. Kang S-HL, Shen J, Belmont J W, et al. (2010) Recurrent reciprocal 16p11.2 rearrangements associated with global developmental delay, behavioural problems, dysmorphism, epilepsy, and abnormal head size. J Med Genet 47: 332-341. doi:10.1136/jmg.2009.073015.
25. Shen Y, Dies K A, Holm I A, Bridgemohan C, Sobeih M M, et al. (2010) Clinical Genetic Testing for Patients With Autism Spectrum Disorders. Pediatrics 125: e727-e735. doi:10.1542/peds.2009-1684.
26. Fernandez B A, Roberts W, Chung B, Weksberg R, Meyn S, et al. (2010) Phenotypic spectrum associated with de novo and inherited deletions and duplications at 16p11.2 in individuals ascertained for diagnosis of autism spectrum disorder. J Med Genet 47: 195-203. doi:10.1136/jmg.2009.069369.
27. Lionel A C, Crosbie J, Barbosa N, Goodale T, Thiruvahindrapuram B, et al. (2011) Rare copy number variation discovery and cross-disorder comparisons identify risk genes for ADHD. Sci Transl Med 3: 95ra75. doi:10.1126/scitranslmed.3002464.
28. Sahoo T, Theisen A, Rosenfeld J A, Lamb A N, Ravnan J B, et al. (2011) Copy number variants of schizophrenia susceptibility loci are associated with a spectrum of speech and developmental delays and behavior problems. Genet Med 13: 868-880. doi:10.1097/GIM.0b013e3182217a06.
29. Kirov G, Pocklington A J, Holmans P, Ivanov D, Ikeda M, et al. (2012) De novo CNV analysis implicates specific abnormalities of postsynaptic signalling complexes in the pathogenesis of schizophrenia. Mol Psychiatry 17: 142-153. doi:10.1038/mp.2011.154.
30. Manning M, Hudgins L (2010) Array-based technology and recommendations for utilization in medical genetics practice for detection of chromosomal abnormalities. Genet Med 12: 742-745. doi: 10.1097/GIM.0b013e3181f8baad.
31. Miller D T, Adam M P, Aradhya S, Biesecker L G, Brothman A R, et al. (2010) Consensus Statement: Chromosomal Microarray Is a First-Tier Clinical Diagnostic Test for Individuals with Developmental Disabilities or Congenital Anomalies. Am J Hum Genet 86: 749-764. doi: 10.1016/j.ajhg.2010.04.006.
32. Glessner J T, Wang K, Cai G, Korvatska O, Kim C E, et al. (2009) Autism genome-wide copy number variation reveals ubiquitin and neuronal genes. Nature 459: 569-573. doi:10.1038/nature07953.
33. Qiao Y, Riendeau N, Koochek M, Liu X, Harvard C, et al. (2009) Phenomic determinants of genomic variation in autism spectrum disorders. J Med Genet 46: 680-688. doi:10.1136/jmg.2009.066795.
34. Wang K, Li M, Hadley D, Liu R, Glessner J, et al. (2007) PennCNV: An integrated hidden Markov model designed for high-resolution copy number variation detection in whole-genome SNP genotyping data. Genome Res 17: 1665-1674. doi:10.1101/gr.6861907.
35. Kent W J, Sugnet C W, Furey T S, Roskin K M, Pringle T H, et al. (2002) The human genome browser at UCSC. Genome Res 12: 996-1006. doi:10.1101/gr.229102.
36. Feng J, Schroer R, Yan J, Song W, Yang C. et al. (2006) High frequency of neurexin 1β signal peptide structural variants in patients with autism. Neurosci Lett 409: 10-13. doi:10.1016/j.neulet.2006.08.017.
37. Kim H-G, Kishikawa S, Higgins A W, Seong I-S, Donovan D J, et al. (2008) Disruption of Neurexin 1 Associated with Autism Spectrum Disorder. Am J Hum Genet 82: 199-207.
38. Ching M S L, Shen Y, Tan W-H, Jeste S S, Morrow E M, et al. (2010) Deletions of NRXN1 (neurexin-1) predispose to a wide spectrum of developmental disorders. Am J Med Genet B Neuropsychiatr Genet 153B: 937-947. doi:10.1002/ajmg.b.31063.
39. Schaaf C P, Boone P M, Sampath S, Williams C, Bader P I, et al. (2012) Phenotypic spectrum and genotype-phenotype correlations of NRXN1 exon deletions. Eur J Hum Genet. Available:http://dx.doi.org/10.1038/ejhg.2012.95.
40. Camacho-Garcia R J, Planelles M I, Margalef M, Pecero M L, Martinez-Leal R. et al. (2012) Mutations affecting synaptic levels of neurexin-1β in autism and mental retardation. Neurobiol Dis 47: 135-143. doi:10.1016/j.nbd.2012.03.031.
41. Wu Y-W, Prakash K, Rong T-Y, Li H-H, Xiao Q, et al. (2011) Lingo2 variants associated with essential tremor and Parkinson's disease. Hum Genet 129: 611-615. doi:10.1007/s00439-011-0955-3.
42. Yamamoto Y. Mochida S, Miyazaki N. Kawai K, Fujikura K, et al. (2010) Tomosyn Inhibits Synaptotagmin-1-mediated Step of Ca2+-dependent Neurotransmitter Release through Its N-terminal WD40 Repeats. J Biol Chem 285: 40943-40955. doi:10.1074/jbc.M110.156893.
43. Williams A L, Bielopolski N, Meroz D, Lam A D, Passmore D R, et al. (2011) Structural and Functional Analysis of Tomosyn Identifies Domains Important in Exocytotic Regulation. J Biol Chem 286: 14542-14553. doi:10.1074/jbc.M110.215624.
44. Hedges D, Hamilton-Nelson K, Sacharow S, Nations L, Beecham G, et al. (2012) Evidence of novel fine-scale structural variation at autism spectrum disorder candidate loci. Mol Autism 3:2. doi: 10.1186/2040-2392-3-2.
45. Nunn C, Mao H, Chidiac P, Albert PR (2006) RGS17/RGSZ2 and the RZ/A family of regulators of G-protein signaling. Semin Cell Dev Biol 17: 390-399. doi:10.1016/j.semcdb.2006.04.001.
46. Shema E, Kim J. Roeder R G, Oren M (2011) RNF20 inhibits TFIIS-facilitated transcriptional elongation to suppress pro-oncogenic gene expression. Mol Cell 42: 477-488. doi:10.1016/j.molce1.2011.03.011.
47. Carrie A, Jun L, Bienvenu T, Vinet M C, McDonell N, et al. (1999) A new member of the IL-1 receptor family highly expressed in hippocampus and involved in X-linked mental retardation. Nat Genet 23: 25-31. doi:10.1038/12623.
48. Gambino F, Pavlowsky A, Béglé A, Dupont J-L, Bahi N, et al. (2007) IL1-receptor accessory protein-like 1 (IL1RAPL1), a protein involved in cognitive functions, regulates N-type Ca2+-channel and neurite elongation. Proc Natl Acad Sci USA 104: 9063-9068. doi:10.1073/pnas.0701133104.
49. Biswas A K, Johnson D G (2012) Transcriptional and nontranscriptional functions of E2F1 in response to DNA damage. Cancer Res 72: 13-17. doi:10.1158/0008-5472.CAN-11-2196.
50. Sumioka A, Imoto S, Martins R N, Kirino Y, Suzuki T (2003) XB51 isoforms mediate Alzheimer's beta-amyloid peptide production by X11L (X11-like protein)-dependent and -independent mechanisms. Biochem J 374: 261-268. doi:10.1042/BJ20030489.
51. Stone T W, Forrest C M, Darlington L G (2012) Kynurenine pathway inhibition as a therapeutic strategy for neuroprotection. FEBS J 279: 1386-1397. doi:10.1111/j.1742-4658.2012.08487.x.
52. Sun J, Jayathilake K, Zhao Z, Meltzer H Y (n.d.) Investigating association of four gene regions (GABRB3, MAOB, PAH, and SLC6A4) with five symptoms in schizophrenia. Psychiatry Res. Available:http://www.sciencedirect.com/science/article/pii/S0165178111008195.
53. Yalçin Ö (2012) Genes and molecular mechanisms involved in the epileptogenesis of idiopathic absence epilepsies. Seizure 21: 79-86. doi: 10.1016/j.seizure.2011.12.002.
54. Kirov G, Rujescu D, Ingason A, Collier D A, O'Donovan M C, et al. (2009) Neurexin 1 (NRXN1) Deletions in Schizophrenia. Schizophr Bull 35: 851-854. doi:10.1093/schbul/sbp079.
55. Harrison V, Connell L, Hayesmoore J, McParland J, Pike M G, et al. (2011) Compound heterozygous deletion of NRXN1 causing severe developmental delay with early onset epilepsy in two sisters. Am J Med Genet A. 155A: 2826-2831. doi:10.1002/ajmg.a.34255.
56. Kalia L V, Kalia S K, Chau H, Lozano A M, Hyman B T, et al. (2011) Ubiquitinylation of α-Synuclein by Carboxyl Terminus Hsp70-Interacting Protein (CHIP) Is Regulated by Bc1-2-Associated Athanogene 5 (BAGS). PLoS ONE 6: e14695. doi:10.1371/journal.pone.0014695.
57. Swaminathan S, Kim S, Shen L, Risacher S L, Foroud T (2011) Genomic Copy Number Analysis in Alzheimer's Disease and Mild Cognitive Impairment: An ADN1 Study. Int J Alzheimers Dis 2011: 10. doi:10.4061/2011/729478.
58. Håvik B, Le Hellard S, Rietschel M, Lybæk H, Djurovic S, et al. (2011) The Complement Control-Related Genes CSMD1 and CSMD2 Associate to Schizophrenia. Biol Psychiatry 70: 35-42. doi: 10.1016/j.biopsych.2011.01.030.
59. Vilariño-Güell C, Wider C, Ross O, Jasinska-Myga B, Kachergus J, et al. (2010) LINGO1 and LINGO2 variants are associated with essential tremor and Parkinson disease. Neurogenetics 11: 401-408. doi:10.1007/s10048-010-0241-x.
60. Punia S, Das M, Behari M, Mishra B K, Sahani A K, et al. (2010) Role of polymorphisms in dopamine synthesis and metabolism genes and association of DBH haplotypes with Parkinson's disease among North Indians. Pharmacogenet Genomics 20:435-441. doi:10.1097/FPC.0b013e32833ad3bb
61. Kao W-T, Wang Y, Kleinman J E, Lipska B K, Hyde T M, et al. (2010) Common genetic variation in Neuregul in 3 (NRG3) influences risk for schizophrenia and impacts NRG3 expression in human brain. Proc Natl Acad Sci USA 107: 15619-15624. doi:10.1073/pnas.1005410107.
62. Grant SG (2012) Synaptopathies: diseases of the synaptome. Curr Opin Neurobiol 22:522-529. Available:http://www.sciencedirect.com/science/article/pii/S0959438812000244.
63. Michel M, Schmidt M J, Mimics K (2012) Immune system gene dysregulation in autism & schizophrenia. Dev Neurobiol. Available:http://www.ncbi.nlm.nih.gov/pubmed/22753382. Accessed 20 Jul. 2012.
64. Davis L K, Meyer K J, Rudd D S, Librant A L, Epping E A, et al. (2009) Novel copy number variants in children with autism and additional developmental anomalies. J Neurodev Disord 1:292-301. doi:10.1007/s11689-009-9013-z.
65. Kang J-Q, Barnes G (n.d.) A Common Susceptibility Factor of Both Autism and Epilepsy: Functional Deficiency of GABA_{A} Receptors. J Autism Dev Disord: 1-12. doi:10.1007/s10803-012-1543-7.
66. Hogart A, Nagarajan R P, Patzel K A, Yasui D H, Lasalle J M (2007) 15q11-13 GABAA receptor genes are normally biallelically expressed in brain yet are subject to epigenetic dysregulation in autism-spectrum disorders. Hum Mol Genet 16: 691-703. doi:10.1093/hmg/ddm014.
67. Cook E H Jr, Lindgren V, Leventhal B L, Courchesne R, Lincoln A, et al. (1997) Autism or atypical autism in maternally but not paternally derived proximal 15q duplication. Am J Hum Genet 60: 928-934.
68. Xu L, Li Y, Zhang X, Sun H, Sun D, et al. (2011) Deletion of LCE3C and LCE3B genes is associated with psoriasis in a northern Chinese population. Br J Dermatol 165: 882-887. doi:10.1111/j.1365-2133.2011.10485.x.
69. Bergboer J G M, Zeeuwen P L J M, Schalkwijk J (2012) Genetics of Psoriasis: Evidence for Epistatic Interaction between Skin Barrier Abnormalities and Immune Deviation. The J Invest Dermatol. Available:http://www.ncbi.nlm.nih.gov/pubmed/22622420. Accessed 20 Jul. 2012.
70. Prescott S M, Lalouel J M, Leppert M (2008) From Linkage Maps to Quantitative Trait Loci: The History and Science of the Utah Genetic Reference Project. Annu Rev Genom Human Genet 9: 347-358. doi:10.1146/annurev.genom.9.081307.164441.
71. Price A L, Patterson N J, Plenge R M, Weinblatt M E, Shadick N A, et al. (2006) Principal components analysis corrects for stratification in genome-wide association studies. Nat Genet 38: 904-909. doi:10.1038/ng1847.
72. Huang D W, Sherman B T, Lempicki R A (2008) Systematic and integrative analysis of large gene lists using DAVID bioinformatics resources. Nat Protocols 4: 44-57. doi:10.1038/nprot.2008.211.
73. Huang D W, Sherman B T, Lempicki R A (2009) Bioinformatics enrichment tools: paths toward the comprehensive functional analysis of large gene lists. Nucleic Acids Res 37: 1-13. doi:10.1093/nar/gkn923/
74. Ji, Tao-yun; Chia, David; Wang, Jing-min; Wu, ye; Li, Jie; Xiao, Jing; Jiang, Yu-wu (n.d.) Diagnosis and fine localization of deletio . . . [Chin Med J (Engl). 2010]-PubMed-NCBI. Available:http://www.ncbi.nlm.nih.gov/pubmed/20819625.
75. Maas, N M; Van Buggenhout, G; Hannes, F; Thienpont, B; Sanlaville, D; Kok, K; Midro, A; Andrieux, J; Anderlid, B M; Schoumans J; Hordijk, R; Devriendt, K; Fryns, J P; Vermeesch, J R (n.d.) Genotype-phenotype correlation in 21 patients wi . . . [J Med Genet. 2008]-PubMed-NCBI. Available:http://www.ncbi.nlm.nih.gov/pubmed/17873117
76. Weise A el al. (2012) Microdeletion and Microduplication Syndromes. Journal Histochemistry & Cytochemistry 60(5) 346-358.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for assessing the presence or absence of a chromosomal deletion or duplication syndrome in a subject, comprising:
probing a sample obtained from the subject for the presence or absence of one or more copy number variants (CNVs) associated with the chromosomal deletion or duplication syndrome, wherein the probing step comprises,
mixing the sample with five or more oligonucleotides that are substantially complementary to portions of the genomic DNA sequence associated with the deletion or duplication syndrome under conditions suitable for hybridization of the five or more oligonucleotides to their complements or substantial complements;
detecting whether hybridization occurs between the five or more oligonucleotides to their complements or substantial complements, or a subset thereof;
obtaining hybridization values of the sample based on the detecting step;
comparing the hybridization values of the sample to reference hybridization value(s) from at least one training set comprising hybridization value(s) from a sample that is positive for the one or more CNVs, or hybridization value(s) from a sample that is negative for the one or more CNVs, wherein the comparing step comprises determining a correlation between the hybridization values obtained from the sample and the hybridization value(s) from the at least one training set;
determining whether the one or more CNV(s) is present or absent based on the comparing step;
assessing whether the subject has the chromosomal deletion or duplication syndrome based on the determination of whether the one or more CNV(s) is present or absent.

2. The method of claim 1, wherein the chromosomal deletion or duplication syndrome is selected from the syndromes set forth in Table A and Table B.

3. The method of claim 1, wherein the chromosomal region associated with the deletion or duplication syndrome is selected from one of the chromosomal locations set forth in Table A or Table B.

4. The method of claim 1, wherein the chromosomal deletion or duplication syndrome is associated with deletion or duplication of a mitochondrial associated gene.

5. The method of claim 4, wherein the mitochondrial associated gene is selected from one or more of the genes in Table 15.

6. The method of any one of claims 1-5, wherein the five or more oligonucleotides comprise from about 20 to about 2,000 oligonucleotides, from about 20 to about 1,500 oligonucleotides, from about 20 to about 1,000 oligonucleotides, from about 20 to about 750 oligonucleotides, from about 20 to about 500 oligonucleotides, from about 20 to about 250 oligonucleotides, or from about 20 to about 100 oligonucleotides.

7. The method of any one of claims 1-5, wherein the five or more oligonucleotides comprise 20 or more oligonucleotides, 25 or more oligonucleotides, 30 or more oligonucleotides or 50 or more oligonucleotides.

8. The method of any one of claims 1-7, wherein the sample comprises restriction digested double stranded DNA obtained from genomic DNA fragments; restriction digested single stranded DNA obtained from genomic DNA fragments; amplified restriction digested genomic DNA single stranded fragments; amplified restriction digested genomic DNA double stranded fragments; or a combination thereof.

9. The method of claim 8, wherein the sample is free of histone proteins.

10. The method of claim 8 or 9, wherein the amplified restriction digested genomic DNA single stranded fragments comprise a detectable label chemically attached to individual single stranded fragments.

11. The method of any one of claims 8-10, wherein the amplified restriction digested genomic DNA single stranded fragments further comprise adapter sequences.

12. The method of claim 11, wherein the adapter sequences are introduced via adapter-specific primers.

13. The method of any one of claims 1-12, further comprising selecting the subject for chromosomal deletion or duplication syndrome therapy.

14. The method of any one of claims 1-13, further comprising measuring the size of the one or more CNVs if the one or more CNVs is present in the sample obtained from the subject.

15. The method of any one of claims 1-14, wherein the five or more oligonucleotides are bound to a solid state substrate.

16. The method of claim 15, wherein the solid state substrate is a glass slide, a silicon wafer or a bead.

17. The method of any one of claims 1-16, further comprising measuring the size of the one or more CNVs if the one or more CNVs is present in the sample obtained from the subject.

18. The method of claim 17, comprising selecting the subject for therapy if the CNV is present, and is at least about 500 bases in length.

19. The method of any one of claims 1-18, wherein the one or more CNVs comprise five to fifty CNVs set forth in Table 15.

20. The method of claim 13 or 18, wherein the subject is selected for treatment with gene therapy, RNA interference (RNAi), behavioral therapy, music therapy, physical therapy, occupational therapy, sensory integration therapy, speech therapy, the Picture Exchange Communication System (PECS), dietary treatment, or drug therapy.

21. The method of claim 20, wherein the behavioral therapy is selected from Applied Behavior Analysis (ABA), Discrete Trial Training (DTT), Early Intensive Behavioral Intervention (EIBI), Pivotal Response Training (PRT), Verbal Behavior Intervention (VBI), and Developmental Individual Differences Relationship-Based Approach (DIR), or a combination thereof.

22. The method of claim 20, wherein the drug therapy is selected from antipsychotics, anti-depressants, anticonvulsants, stimulants, aripiprazole, guanfacine, selective serotonin reuptake inhibitors (SSRIs), riseridone, olanzapine, naltrexone, or a combination thereof.

23. The method of any one of claims 1-18, wherein the chromosomal deletion or duplication syndrome is Wolf-Hirschhorn syndrome (WHS).

24. The method of claim 13 or 18, wherein the one or more CNVs is associated with a mitochondrial associated gene and the therapy comprises administration to the subject EPI-743, antioxidants, Oxygen, arginine, Coenzyme Q10, idebenone, benzoquinone therapeutics, or a combination thereof.

25. The method of claim 13 or 18, wherein the one or more CNVs is associated with a glutamate or GABA receptor gene and the therapy comprises administration to the subject a glutamate receptor agonist or antagonist or a GABA receptor agonist or antagonist.

26. The method of claim 25, wherein the subject is selected for therapy with a glutamatergic receptor agonist or GABAergic antagonist if the effect of the CNV is an inhibitory effect, and wherein the subject is administered a glutamatergic receptor antagonist or GABAergic agonist if the effect of the CNV is an excitatory effect.

27. The method of any one of claims 1-26, wherein the sample comprises polymerase chain reaction (PCR) amplified restriction digested genomic DNA single stranded fragments.

28. The method of claim 27, wherein the PCR amplified restriction digested genomic DNA single stranded fragments comprise a detectable label chemically attached to individual single stranded fragments.

29. The method of claim 28, wherein the amplified restriction digested genomic DNA single stranded fragments further comprise adapter sequences.

30. The method of claim 29, wherein the adapter sequences are introduced via adapter-specific primers.

33. The method of any one of claims 28-30, wherein the detectable label is a fluorescent label, enzyme label, radioisotope, chemiluminescent label, electrochemiluminescent label, bioluminescent label, polymer, polymer particle, metal particle, hapten, dye, or a combination thereof.

34. The method of claim 33, wherein the detectable label is a fluorescent label.

35. The method of claim 23, comprising selecting the patient for therapy if the deletion on the 4p chromosome is greater than or equal to 500 bases in length.

36. The method of claim 23, comprising selecting the patient for therapy if the deletion on the 4p chromosome is greater than or equal to 1000 bases in length.

37. The method of claim 23, comprising selecting the patient for therapy if the deletion on the 4p chromosome is greater than or equal to 1 Mb in length.

28. The method of claim 34, wherein the fluorescent label is selected from 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, conjugates of R-phycoerythrin or allophycoerythrin, inorganic fluorescent labels such as particles based on semiconductor material like coated CdSe nanocrystallites, or a combination thereof.
